SEQUENCE LISTING (590 sequences)

SEQ ID NO: 1 - DNA, 42 bp, Artificial Sequence
  Template sequence
tgctgaatattcagattctctagtgctacctgaaagatcctg

SEQ ID NO: 2 - DNA, 42 bp, Artificial Sequence
  Template sequence
tgctgaatattcagattctcgagtgctacctgaaagatcctg

SEQ ID NO: 3 - DNA, 41 bp, Artificial Sequence
  Template sequence
caccatgctcacaatgaatgcaggatctttcaggtagcact

SEQ ID NO: 4 - DNA, 41 bp, Artificial Sequence
  Template sequence
gataattctctgataggccgcaggatctttcaggtagcact

SEQ ID NO: 5 - DNA, 41 bp, Artificial Sequence
  Template sequence
gactacgatgtgatccgtgtcaggatctttcaggtagcact

SEQ ID NO: 6 - DNA, 41 bp, Artificial Sequence
  Template sequence
gaacgcagttatcagactctcaggatctttcaggtagcact

SEQ ID NO: 7 - DNA, 41 bp, Artificial Sequence
  Template sequence
cgaggacatggagtcacatccaggatctttcaggtagcact

SEQ ID NO: 8 - DNA, 41 bp, Artificial Sequence
  Template sequence
gctaggcattcctccagtgtcaggatctttcaggtagcact

SEQ ID NO: 9 - DNA, 41 bp, Artificial Sequence
  Template sequence
tgtaaaacgacggccagttaatacgactcactatagggaga

SEQ ID NO: 10 - DNA, 39 bp, Artificial Sequence
  Template sequence
aacagctatgaccatgaattaaccctcactaaagggaga

SEQ ID NO: 11 - DNA, 16 bp, Artificial Sequence
  Template sequence
aacagctatgaccatg

SEQ ID NO: 12 - DNA, 23 bp, Artificial Sequence
  Template sequence
taatacgactcactatagggaga

SEQ ID NO: 13 - DNA, 13 bp, Artificial Sequence
  Template sequence
atgctatcannnn

SEQ ID NO: 14 - DNA, 8 bp, Artificial Sequence
  Template sequence
gcatgcat

SEQ ID NO: 15 - DNA, 9 bp, Artificial Sequence
  Template sequence
tgcatgcat

SEQ ID NO: 16 - DNA, 21 bp, Artificial Sequence
  Template sequence
ttccgaggaakggcagaatgg

SEQ ID NO: 17 - DNA, 21 bp, Artificial Sequence
  Template sequence
cagaagggctstgaaggtgag

SEQ ID NO: 18 - DNA, 21 bp, Artificial Sequence
  Template sequence
tgctggtcccyagccaggagg

SEQ ID NO: 19 - DNA, 21 bp, Artificial Sequence
  Template sequence
ccaggaggcaycccaacaggt

SEQ ID NO: 20 - DNA, 21 bp, Artificial Sequence
  Template sequence
aacaaccagcrgccagacaac

SEQ ID NO: 21 - DNA, 21 bp, Artificial Sequence
  Template sequence
aacgggcagcrctgcctgccc

SEQ ID NO: 22 - DNA, 21 bp, Artificial Sequence
  Template sequence
agccattcaamcccctaccag

SEQ ID NO: 23 - DNA, 21 bp, Artificial Sequence
  Template sequence
tgatggccacrtcccggaaat

SEQ ID NO: 24 - DNA, 21 bp, Artificial Sequence
  Template sequence
cactctacctsaacctgcatg

SEQ ID NO: 25 - DNA, 21 bp, Artificial Sequence
  Template sequence
catgaggccaytggggacgtg

SEQ ID NO: 26 - DNA, 21 bp, Artificial Sequence
  Template sequence
tgacatcaackttctgatgaa

SEQ ID NO: 27 - DNA, 21 bp, Artificial Sequence
  Template sequence
ccaaggaggcygggcagcgcc

SEQ ID NO: 28 - DNA, 21 bp, Artificial Sequence
  Template sequence
ccaggtacttygtcagcttca

SEQ ID NO: 29 - DNA, 21 bp, Artificial Sequence
  Template sequence
ctcagccagcrgctcttcagc

SEQ ID NO: 30 - DNA, 21 bp, Artificial Sequence
  Template sequence
catccgccacmgcagcctcca

SEQ ID NO: 31 - DNA, 21 bp, Artificial Sequence
  Template sequence
cacgggccccmgttcggctcc

SEQ ID NO: 32 - DNA, 21 bp, Artificial Sequence
  Template sequence
ccgaggtggarctgagacact

SEQ ID NO: 33 - DNA, 21 bp, Artificial Sequence
  Template sequence
aggatctgccygtctccctgc

SEQ ID NO: 34 - DNA, 21 bp, Artificial Sequence
  Template sequence
taagggggggytgctgtacat

SEQ ID NO: 35 - DNA, 21 bp, Artificial Sequence
  Template sequence
tatagtatatmtatgcccagc

SEQ ID NO: 36 - DNA, 21 bp, Artificial Sequence
  Template sequence
gctggggactytggagcggag

SEQ ID NO: 37 - DNA, 21 bp, Artificial Sequence
  Template sequence
agttcttcagygttgccctcc

SEQ ID NO: 38 - DNA, 42 bp, Artificial Sequence
  Template sequence
ctcagaggacraccccgagtaggccatcgccyggactccgag

SEQ ID NO: 39 - DNA, 21 bp, Artificial Sequence
  Template sequence
ggatgtccagsagctacccca

SEQ ID NO: 40 - DNA, 20 bp, Artificial Sequence
  Template sequence
cttctttgccwgatgctgcg

SEQ ID NO: 41 - DNA, 21 bp, Artificial Sequence
  Template sequence
tcagctcacgmcaccgaggca

SEQ ID NO: 42 - DNA, 21 bp, Artificial Sequence
  Template sequence
gggtacccacraggtgaggac

SEQ ID NO: 43 - DNA, 21 bp, Artificial Sequence
  Template sequence
ccgtttctccytggtctaagt

SEQ ID NO: 44 - DNA, 21 bp, Artificial Sequence
  Template sequence
ggatgctggcyaacttcttgg

SEQ ID NO: 45 - DNA, 21 bp, Artificial Sequence
  Template sequence
ttcacagaackggatgttgct

SEQ ID NO: 46 - DNA, 21 bp, Artificial Sequence
  Template sequence
tgctccctgaygggagccagt

SEQ ID NO: 47 - DNA, 21 bp, Artificial Sequence
  Template sequence
ggaaagcagcsctttctcctt

SEQ ID NO: 48 - DNA, 21 bp, Artificial Sequence
  Template sequence
ccgtttctccytggtctaagt

SEQ ID NO: 49 - DNA, 21 bp, Artificial Sequence
  Template sequence
tcggtttgtakttagtgtctt

SEQ ID NO: 50 - DNA, 21 bp, Artificial Sequence
  Template sequence
ctgtgctattsttggtgttta

SEQ ID NO: 51 - DNA, 21 bp, Artificial Sequence
  Template sequence
ccttggccccractcctgcaa

SEQ ID NO: 52 - DNA, 21 bp, Artificial Sequence
  Template sequence
accctgcaccrgctcactctg

SEQ ID NO: 53 - DNA, 21 bp, Artificial Sequence
  Template sequence
ctgcacctccrgcctgcatgt

SEQ ID NO: 54 - DNA, 21 bp, Artificial Sequence
  Template sequence
taaatagggcmtcgtgacccg

SEQ ID NO: 55 - DNA, 21 bp, Artificial Sequence
  Template sequence
tgacccggccrggggaagaag

SEQ ID NO: 56 - DNA, 21 bp, Artificial Sequence
  Template sequence
aagaagctgcygttgttctgg

SEQ ID NO: 57 - DNA, 21 bp, Artificial Sequence
  Template sequence
caagatgcccwtcctggggtt

SEQ ID NO: 58 - DNA, 21 bp, Artificial Sequence
  Template sequence
gtacgcgccgsggccaaggcc

SEQ ID NO: 59 - DNA, 21 bp, Artificial Sequence
  Template sequence
tcgctggcttwgctgtggtgc

SEQ ID NO: 60 - DNA, 21 bp, Artificial Sequence
  Template sequence
tttgcagtacygaagataaca

SEQ ID NO: 61 - DNA, 21 bp, Artificial Sequence
  Template sequence
tcctgttgcaytcaagtccaa

SEQ ID NO: 62 - DNA, 21 bp, Artificial Sequence
  Template sequence
aggaacagcakcaggagcagc

SEQ ID NO: 63 - DNA, 21 bp, Artificial Sequence
  Template sequence
ttctgtcgatsgtcttggaag

SEQ ID NO: 64 - DNA, 21 bp, Artificial Sequence
  Template sequence
ctttctccccwgggacttgta

SEQ ID NO: 65 - DNA, 21 bp, Artificial Sequence
  Template sequence
cttgtacagcmaaagcacagc

SEQ ID NO: 66 - DNA, 21 bp, Artificial Sequence
  Template sequence
ctgggtccgckcaccaaggcc

SEQ ID NO: 67 - DNA, 21 bp, Artificial Sequence
  Template sequence
actgtccagcmttgacttcag

SEQ ID NO: 68 - DNA, 21 bp, Artificial Sequence
  Template sequence
gggaagaacargataacccgt

SEQ ID NO: 69 - DNA, 21 bp, Artificial Sequence
  Template sequence
aactacctgcygtcgccctgc

SEQ ID NO: 70 - DNA, 21 bp, Artificial Sequence
  Template sequence
gcggagctggygctgctctcc

SEQ ID NO: 71 - DNA, 21 bp, Artificial Sequence
  Template sequence
cccatgctggygtaccgccat

SEQ ID NO: 72 - DNA, 21 bp, Artificial Sequence
  Template sequence
ggcactttgayggtgttgcca

SEQ ID NO: 73 - DNA, 21 bp, Artificial Sequence
  Template sequence
tacctgggctyggcagggtcc

SEQ ID NO: 74 - DNA, 21 bp, Artificial Sequence
  Template sequence
catctatattmcttatgctgt

SEQ ID NO: 75 - DNA, 21 bp, Artificial Sequence
  Template sequence
ttccctgcagmctggacagcc

SEQ ID NO: 76 - DNA, 21 bp, Artificial Sequence
  Template sequence
gacccgacacsagccattact

SEQ ID NO: 77 - DNA, 21 bp, Artificial Sequence
  Template sequence
catggcccacrggaattctac

SEQ ID NO: 78 - DNA, 21 bp, Artificial Sequence
  Template sequence
acggctgctcrttgtgcagga

SEQ ID NO: 79 - DNA, 21 bp, Artificial Sequence
  Template sequence
ggccaccttgsttctcgcctc

SEQ ID NO: 80 - DNA, 21 bp, Artificial Sequence
  Template sequence
gccaaggacaygccactggag

SEQ ID NO: 81 - DNA, 21 bp, Artificial Sequence
  Template sequence
aatcccggagstgaagaccat

SEQ ID NO: 82 - DNA, 21 bp, Artificial Sequence
  Template sequence
ggatatcaagmactttggggc

SEQ ID NO: 83 - DNA, 21 bp, Artificial Sequence
  Template sequence
ccaattgtcakacgacttgca

SEQ ID NO: 84 - DNA, 21 bp, Artificial Sequence
  Template sequence
atggtagaagytggagcacca

SEQ ID NO: 85 - DNA, 21 bp, Artificial Sequence
  Template sequence
aggagcacttkgaggcctggg

SEQ ID NO: 86 - DNA, 21 bp, Artificial Sequence
  Template sequence
cgtggcggagstcctgttgaa

SEQ ID NO: 87 - DNA, 21 bp, Artificial Sequence
  Template sequence
caggccctgargaagaaggtg

SEQ ID NO: 88 - DNA, 21 bp, Artificial Sequence
  Template sequence
gtgcagcagaycctgcgccag

SEQ ID NO: 89 - DNA, 21 bp, Artificial Sequence
  Template sequence
gagcgagtggygagctcagac

SEQ ID NO: 90 - DNA, 21 bp, Artificial Sequence
  Template sequence
gccctcagacrcgtgcaccat

SEQ ID NO: 91 - DNA, 21 bp, Artificial Sequence
  Template sequence
gatgaaacccraccgcctcaa

SEQ ID NO: 92 - DNA, 21 bp, Artificial Sequence
  Template sequence
agccggccttkccttcggggg

SEQ ID NO: 93 - DNA, 21 bp, Artificial Sequence
  Template sequence
ctcagaggacraccccgagta

SEQ ID NO: 94 - DNA, 21 bp, Artificial Sequence
  Template sequence
gatataattaygatggactac

SEQ ID NO: 95 - DNA, 21 bp, Artificial Sequence
  Template sequence
gactttctgcygctggactct

SEQ ID NO: 96 - DNA, 21 bp, Artificial Sequence
  Template sequence
ttgggacaacragaagccaac

SEQ ID NO: 97 - DNA, 21 bp, Artificial Sequence
  Template sequence
gtgggacaacsagaagcccac

SEQ ID NO: 98 - DNA, 21 bp, Artificial Sequence
  Template sequence
ccccagatgakcccccagaac

SEQ ID NO: 99 - DNA, 21 bp, Artificial Sequence
  Template sequence
tgaaaggcaakccctccagag

SEQ ID NO: 100 - DNA, 21 bp, Artificial Sequence
  Template sequence
gcacgcagccsctccgggagc

SEQ ID NO: 101 - DNA, 21 bp, Artificial Sequence
  Template sequence
tcagaaggtcscggcgcaaag

SEQ ID NO: 102 - DNA, 21 bp, Artificial Sequence
  Template sequence
aacatgggctkctggtggatc

SEQ ID NO: 103 - DNA, 21 bp, Artificial Sequence
  Template sequence
agcactaattmtctgtggagc

SEQ ID NO: 104 - DNA, 21 bp, Artificial Sequence
  Template sequence
cagtgtgcctyccatgctcca

SEQ ID NO: 105 - DNA, 21 bp, Artificial Sequence
  Template sequence
ggcaccctcascaccctctgg

SEQ ID NO: 106 - DNA, 21 bp, Artificial Sequence
  Template sequence
tttctcttgtracaatggctt

SEQ ID NO: 107 - DNA, 21 bp, Artificial Sequence
  Template sequence
ccactggtcamctaccgtgcc

SEQ ID NO: 108 - DNA, 21 bp, Artificial Sequence
  Template sequence
tactccagggktgcaatgtcc

SEQ ID NO: 109 - DNA, 21 bp, Artificial Sequence
  Template sequence
gaagcccccartgaaggaacc

SEQ ID NO: 110 - DNA, 21 bp, Artificial Sequence
  Template sequence
catcatctgcrgcatcacgtc

SEQ ID NO: 111 - DNA, 21 bp, Artificial Sequence
  Template sequence
catccgtgcascagagtggcc

SEQ ID NO: 112 - DNA, 21 bp, Artificial Sequence
  Template sequence
ggagcgctggragggagagct

SEQ ID NO: 113 - DNA, 21 bp, Artificial Sequence
  Template sequence
aacggcagcgrgcagacagtg

SEQ ID NO: 114 - DNA, 21 bp, Artificial Sequence
  Template sequence
atgttgggctsaggacccagc

SEQ ID NO: 115 - DNA, 21 bp, Artificial Sequence
  Template sequence
ttccctggacrggctgttccc

SEQ ID NO: 116 - DNA, 21 bp, Artificial Sequence
  Template sequence
ttgcaacctcmgcctcgctat

SEQ ID NO: 117 - DNA, 21 bp, Artificial Sequence
  Template sequence
gaccagcccawgttgttgggc

SEQ ID NO: 118 - DNA, 21 bp, Artificial Sequence
  Template sequence
ggtcacccgcraggtgaccgt

SEQ ID NO: 119 - DNA, 21 bp, Artificial Sequence
  Template sequence
gatggcccccractggacgag

SEQ ID NO: 120 - DNA, 21 bp, Artificial Sequence
  Template sequence
aaagaagctgrcggttgagcc

SEQ ID NO: 121 - DNA, 21 bp, Artificial Sequence
  Template sequence
cagggcccggrcgccgcctca

SEQ ID NO: 122 - DNA, 21 bp, Artificial Sequence
  Template sequence
gttgcccaccsaggaacccga

SEQ ID NO: 123 - DNA, 21 bp, Artificial Sequence
  Template sequence
gtccagaaggwgacagacttc

SEQ ID NO: 124 - DNA, 21 bp, Artificial Sequence
  Template sequence
ccagtgtcagyttggtaagtc

SEQ ID NO: 125 - DNA, 21 bp, Artificial Sequence
  Template sequence
tatgagatcartaaagtcaga

SEQ ID NO: 126 - DNA, 21 bp, Artificial Sequence
  Template sequence
aataagaagtsaggctggtga

SEQ ID NO: 127 - DNA, 21 bp, Artificial Sequence
  Template sequence
tgctggtcccyagccaggagg

SEQ ID NO: 128 - DNA, 21 bp, Artificial Sequence
  Template sequence
catgcgcgccrttgttaaaag

SEQ ID NO: 129 - DNA, 21 bp, Artificial Sequence
  Template sequence
caggtgaccaytgacggcagg

SEQ ID NO: 130 - DNA, 21 bp, Artificial Sequence
  Template sequence
agtcctgcctkcctcctggtg

SEQ ID NO: 131 - DNA, 21 bp, Artificial Sequence
  Template sequence
ctacgacgacyacatcttccc

SEQ ID NO: 132 - DNA, 21 bp, Artificial Sequence
  Template sequence
aatggcatcamtgcctacctg

SEQ ID NO: 133 - DNA, 21 bp, Artificial Sequence
  Template sequence
tggttacatgscccatgaaca

SEQ ID NO: 134 - DNA, 21 bp, Artificial Sequence
  Template sequence
cacaaggtcarcattgaggat

SEQ ID NO: 135 - DNA, 21 bp, Artificial Sequence
  Template sequence
caccagcttcstctctgggtt

SEQ ID NO: 136 - DNA, 21 bp, Artificial Sequence
  Template sequence
tgcataaccarggtgagtagg

SEQ ID NO: 137 - DNA, 21 bp, Artificial Sequence
  Template sequence
gatcaatgacrtttcacacac

SEQ ID NO: 138 - DNA, 21 bp, Artificial Sequence
  Template sequence
gtaattttccrgtcacctcta

SEQ ID NO: 139 - DNA, 21 bp, Artificial Sequence
  Template sequence
gggagcagggkttctcccaga

SEQ ID NO: 140 - DNA, 21 bp, Artificial Sequence
  Template sequence
gccgccgccgmcagcggcatc

SEQ ID NO: 141 - DNA, 21 bp, Artificial Sequence
  Template sequence
tgctggacaamccgtacaccc

SEQ ID NO: 142 - DNA, 21 bp, Artificial Sequence
  Template sequence
ggaggctgtgwgcccagatct

SEQ ID NO: 143 - DNA, 21 bp, Artificial Sequence
  Template sequence
ggggggcaccwcctcctcatc

SEQ ID NO: 144 - DNA, 21 bp, Artificial Sequence
  Template sequence
atcggggcccwggaggagtcg

SEQ ID NO: 145 - DNA, 21 bp, Artificial Sequence
  Template sequence
ccctacttacratcctgggag

SEQ ID NO: 146 - DNA, 21 bp, Artificial Sequence
  Template sequence
aacattctatstgagaagcct

SEQ ID NO: 147 - DNA, 21 bp, Artificial Sequence
  Template sequence
caccaactttrgcttccagcc

SEQ ID NO: 148 - DNA, 21 bp, Artificial Sequence
  Template sequence
cagcctgacayggccccccgc

SEQ ID NO: 149 - DNA, 21 bp, Artificial Sequence
  Template sequence
acctgcattgkcatgtgcaag

SEQ ID NO: 150 - DNA, 21 bp, Artificial Sequence
  Template sequence
ggctcccccaygtccagaagc

SEQ ID NO: 151 - DNA, 21 bp, Artificial Sequence
  Template sequence
ttctgtccaamttcggtggcc

SEQ ID NO: 152 - DNA, 21 bp, Artificial Sequence
  Template sequence
gtcgtggccckctccgggcgg

SEQ ID NO: 153 - DNA, 21 bp, Artificial Sequence
  Template sequence
ggtgtcccgckgccgtctgcg

SEQ ID NO: 154 - DNA, 21 bp, Artificial Sequence
  Template sequence
gcggaaagtcrgcggtagcat

SEQ ID NO: 155 - DNA, 21 bp, Artificial Sequence
  Template sequence
ccccgcaggcrctgtgctaga

SEQ ID NO: 156 - DNA, 21 bp, Artificial Sequence
  Template sequence
tgccacctacstactggccac

SEQ ID NO: 157 - DNA, 21 bp, Artificial Sequence
  Template sequence
ctcatggccaycccgctggag

SEQ ID NO: 158 - DNA, 21 bp, Artificial Sequence
  Template sequence
gcagaacttasatgataagca

SEQ ID NO: 159 - DNA, 17 bp, Artificial Sequence
  Template sequence
gacgaagcttccgagga

SEQ ID NO: 160 - DNA, 18 bp, Artificial Sequence
  Template sequence
gagaggaagcagaagggc

SEQ ID NO: 161 - DNA, 17 bp, Artificial Sequence
  Template sequence
gcaccctctgctggtcc

SEQ ID NO: 162 - DNA, 17 bp, Artificial Sequence
  Template sequence
gcaccctctgctggtcc

SEQ ID NO: 163 - DNA, 17 bp, Artificial Sequence
  Template sequence
agccaggcaacaaccag

SEQ ID NO: 164 - DNA, 18 bp, Artificial Sequence
  Template sequence
aggacctagaacgggcag

SEQ ID NO: 165 - DNA, 18 bp, Artificial Sequence
  Template sequence
tggagctcaagccattca

SEQ ID NO: 166 - DNA, 17 bp, Artificial Sequence
  Template sequence
gacgaatgtgatggcca

SEQ ID NO: 167 - DNA, 19 bp, Artificial Sequence
  Template sequence
gagctgcagccactctacc

SEQ ID NO: 168 - DNA, 16 bp, Artificial Sequence
  Template sequence
cggcttccatgaggcc

SEQ ID NO: 169 - DNA, 19 bp, Artificial Sequence
  Template sequence
ttgcagagcatgacatcaa

SEQ ID NO: 170 - DNA, 20 bp, Artificial Sequence
  Template sequence
catctaccagtccaaggagg

SEQ ID NO: 171 - DNA, 19 bp, Artificial Sequence
  Template sequence
tcgctctgctccaggtact

SEQ ID NO: 172 - DNA, 15 bp, Artificial Sequence
  Template sequence
ctgggcctcagccag

SEQ ID NO: 173 - DNA, 16 bp, Artificial Sequence
  Template sequence
tcttcagcatccgcca

SEQ ID NO: 174 - DNA, 15 bp, Artificial Sequence
  Template sequence
cactcccacgggccc

SEQ ID NO: 175 - DNA, 15 bp, Artificial Sequence
  Template sequence
tcggctccgaggtgg

SEQ ID NO: 176 - DNA, 20 bp, Artificial Sequence
  Template sequence
cctgcagtacaaggatctgc

SEQ ID NO: 177 - DNA, 18 bp, Artificial Sequence
  Template sequence
ccactgaggataaggggg

SEQ ID NO: 178 - DNA, 27 bp, Artificial Sequence
  Template sequence
gtcatgccatgtcacatatattatagt

SEQ ID NO: 179 - DNA, 15 bp, Artificial Sequence
  Template sequence
aggaacctcggcccg

SEQ ID NO: 180 - DNA, 18 bp, Artificial Sequence
  Template sequence
ccgtgtgcgagttcttca

SEQ ID NO: 181 - DNA, 18 bp, Artificial Sequence
  Template sequence
tgaccgcttctcagagga

SEQ ID NO: 182 - DNA, 15 bp, Artificial Sequence
  Template sequence
catcgtggccatcgc

SEQ ID NO: 183 - DNA, 17 bp, Artificial Sequence
  Template sequence
ggaactgcggatgtcca

SEQ ID NO: 184 - DNA, 19 bp, Artificial Sequence
  Template sequence
ccggtaccttcttctttgc

SEQ ID NO: 185 - DNA, 19 bp, Artificial Sequence
  Template sequence
ccgacctctggttttcagc

SEQ ID NO: 186 - DNA, 16 bp, Artificial Sequence
  Template sequence
cacacccgggtaccca

SEQ ID NO: 187 - DNA, 20 bp, Artificial Sequence
  Template sequence
gactttgagctggaaagcag

SEQ ID NO: 188 - DNA, 15 bp, Artificial Sequence
  Template sequence
tggtcgggatgctgg

SEQ ID NO: 189 - DNA, 20 bp, Artificial Sequence
  Template sequence
cgctctctggacttcacaga

SEQ ID NO: 190 - DNA, 17 bp, Artificial Sequence
  Template sequence
agactggctgctccctg

SEQ ID NO: 191 - DNA, 20 bp, Artificial Sequence
  Template sequence
cagtttgagctggaaagcag

SEQ ID NO: 192 - DNA, 20 bp, Artificial Sequence
  Template sequence
gactttgagctggaaagcag

SEQ ID NO: 193 - DNA, 18 bp, Artificial Sequence
  Template sequence
gcattgccttcggtttgt

SEQ ID NO: 194 - DNA, 21 bp, Artificial Sequence
  Template sequence
ctttcaatctggctgtgctat

SEQ ID NO: 195 - DNA, 15 bp, Artificial Sequence
  Template sequence
tgggaaccttggccc

SEQ ID NO: 196 - DNA, 20 bp, Artificial Sequence
  Template sequence
tgtgtaactcgaccctgcac

SEQ ID NO: 197 - DNA, 17 bp, Artificial Sequence
  Template sequence
tctgccctctgcacctc

SEQ ID NO: 198 - DNA, 22 bp, Artificial Sequence
  Template sequence
cacccctcagctataaataggg

SEQ ID NO: 199 - DNA, 22 bp, Artificial Sequence
  Template sequence
cacccctcagctataaataggg

SEQ ID NO: 200 - DNA, 17 bp, Artificial Sequence
  Template sequence
gccaggggaagaagctg

SEQ ID NO: 201 - DNA, 15 bp, Artificial Sequence
  Template sequence
acggcgccaagatgc

SEQ ID NO: 202 - DNA, 20 bp, Artificial Sequence
  Template sequence
gctatttaaaggtacgcgcc

SEQ ID NO: 203 - DNA, 15 bp, Artificial Sequence
  Template sequence
gccctctcgctggct

SEQ ID NO: 204 - DNA, 24 bp, Artificial Sequence
  Template sequence
atatgtctgtgttctctttgcagt

SEQ ID NO: 205 - DNA, 19 bp, Artificial Sequence
  Template sequence
ttggaatcctgcttcctgt

SEQ ID NO: 206 - DNA, 18 bp, Artificial Sequence
  Template sequence
agcaacagcaggaacagc

SEQ ID NO: 207 - DNA, 18 bp, Artificial Sequence
  Template sequence
tggtggtggttctgtcga

SEQ ID NO: 208 - DNA, 19 bp, Artificial Sequence
  Template sequence
accatctgtgctttctccc

SEQ ID NO: 209 - DNA, 19 bp, Artificial Sequence
  Template sequence
accatctgtgctttctccc

SEQ ID NO: 210 - DNA, 15 bp, Artificial Sequence
  Template sequence
agggacctgggtccg

SEQ ID NO: 211 - DNA, 21 bp, Artificial Sequence
  Template sequence
caagctacaccaactgtccag

SEQ ID NO: 212 - DNA, 22 bp, Artificial Sequence
  Template sequence
gacgaatagctatgggaagaac

SEQ ID NO: 213 - DNA, 20 bp, Artificial Sequence
  Template sequence
tgccaggaggagaactacct

SEQ ID NO: 214 - DNA, 15 bp, Artificial Sequence
  Template sequence
ctagcccgggcggag

SEQ ID NO: 215 - DNA, 15 bp, Artificial Sequence
  Template sequence
tgccgtcccatgctg

SEQ ID NO: 216 - DNA, 18 bp, Artificial Sequence
  Template sequence
agtggtgtgggcactttg

SEQ ID NO: 217 - DNA, 16 bp, Artificial Sequence
  Template sequence
ggcacggtacctgggc

SEQ ID NO: 218 - DNA, 20 bp, Artificial Sequence
  Template sequence
gaagcattgtgtgccatcta

SEQ ID NO: 219 - DNA, 20 bp, Artificial Sequence
  Template sequence
ctggtatgtgttttccctgc

SEQ ID NO: 220 - DNA, 18 bp, Artificial Sequence
  Template sequence
cgatacgctgacccgaca

SEQ ID NO: 221 - DNA, 21 bp, Artificial Sequence
  Template sequence
ctttgatgagcctcagaagaa

SEQ ID NO: 222 - DNA, 16 bp, Artificial Sequence
  Template sequence
tgtgctgacggctgct

SEQ ID NO: 223 - DNA, 16 bp, Artificial Sequence
  Template sequence
ccgctctggccacctt

SEQ ID NO: 224 - DNA, 16 bp, Artificial Sequence
  Template sequence
ccacactggccaagga

SEQ ID NO: 225 - DNA, 17 bp, Artificial Sequence
  Template sequence
ggttctggaatcccgga

SEQ ID NO: 226 - DNA, 22 bp, Artificial Sequence
  Template sequence
tggaggactacctggatatcaa

SEQ ID NO: 227 - DNA, 19 bp, Artificial Sequence
  Template sequence
cggttagcgaccaattgtc

SEQ ID NO: 228 - DNA, 21 bp, Artificial Sequence
  Template sequence
tttggtgaaaccatggtagaa

SEQ ID NO: 229 - DNA, 19 bp, Artificial Sequence
  Template sequence
aaggtgtggaaggagcact

SEQ ID NO: 230 - DNA, 19 bp, Artificial Sequence
  Template sequence
cagtacaccagcatcgtgg

SEQ ID NO: 231 - DNA, 16 bp, Artificial Sequence
  Template sequence
gcagtggccagggact

SEQ ID NO: 232 - DNA, 15 bp, Artificial Sequence
  Template sequence
cccgacgtgcagcag

SEQ ID NO: 233 - DNA, 20 bp, Artificial Sequence
  Template sequence
gctctaccctgtgggtctgt

SEQ ID NO: 234 - DNA, 17 bp, Artificial Sequence
  Template sequence
ccggagttgccctcaga

SEQ ID NO: 235 - DNA, 19 bp, Artificial Sequence
  Template sequence
gcgactccaagatgaaacc

SEQ ID NO: 236 - DNA, 15 bp, Artificial Sequence
  Template sequence
ccagaggaagccggc

SEQ ID NO: 237 - DNA, 18 bp, Artificial Sequence
  Template sequence
tgacccctattccctgct

SEQ ID NO: 238 - DNA, 25 bp, Artificial Sequence
  Template sequence
ctgaagccataggttttgatataat

SEQ ID NO: 239 - DNA, 21 bp, Artificial Sequence
  Template sequence
ccttggtagctggactttctg

SEQ ID NO: 240 - DNA, 19 bp, Artificial Sequence
  Template sequence
catctgggaattgggacaa

SEQ ID NO: 241 - DNA, 17 bp, Artificial Sequence
  Template sequence
cacaggggagtgggaca

SEQ ID NO: 242 - DNA, 15 bp, Artificial Sequence
  Template sequence
tgcaggccccagatg

SEQ ID NO: 243 - DNA, 17 bp, Artificial Sequence
  Template sequence
tcccaagctgaaaggca

SEQ ID NO: 244 - DNA, 15 bp, Artificial Sequence
  Template sequence
caggtgcagcacgca

SEQ ID NO: 245 - DNA, 20 bp, Artificial Sequence
  Template sequence
cccaccctctctcagaaggt

SEQ ID NO: 246 - DNA, 18 bp, Artificial Sequence
  Template sequence
agcaatgacaacatgggc

SEQ ID NO: 247 - DNA, 22 bp, Artificial Sequence
  Template sequence
ctaaacagaaacaccacagcac

SEQ ID NO: 248 - DNA, 17 bp, Artificial Sequence
  Template sequence
gcagagtgcagtgtgcc

SEQ ID NO: 249 - DNA, 16 bp, Artificial Sequence
  Template sequence
ccctccaggcaccctc

SEQ ID NO: 250 - DNA, 17 bp, Artificial Sequence
  Template sequence
tggagcggtggcttcta

SEQ ID NO: 251 - DNA, 19 bp, Artificial Sequence
  Template sequence
aagagaatggccactggtc

SEQ ID NO: 252 - DNA, 20 bp, Artificial Sequence
  Template sequence
gcatcacttcctactccagg

SEQ ID NO: 253 - DNA, 16 bp, Artificial Sequence
  Template sequence
cagcacctggaagccc

SEQ ID NO: 254 - DNA, 19 bp, Artificial Sequence
  Template sequence
cccacgagagcatcatctg

SEQ ID NO: 255 - DNA, 15 bp, Artificial Sequence
  Template sequence
gcgcaacatccgtgc

SEQ ID NO: 256 - DNA, 15 bp, Artificial Sequence
  Template sequence
ggccctggagcgctg

SEQ ID NO: 257 - DNA, 16 bp, Artificial Sequence
  Template sequence
gcaggtgaacggcagc

SEQ ID NO: 258 - DNA, 16 bp, Artificial Sequence
  Template sequence
tggagcgatgttgggc

SEQ ID NO: 259 - DNA, 18 bp, Artificial Sequence
  Template sequence
cgtggtctgttccctgga

SEQ ID NO: 260 - DNA, 21 bp, Artificial Sequence
  Template sequence
tgctactcagagttgcaacct

SEQ ID NO: 261 - DNA, 18 bp, Artificial Sequence
  Template sequence
cacctcctgtgaccagcc

SEQ ID NO: 262 - DNA, 15 bp, Artificial Sequence
  Template sequence
aggggaggtcacccg

SEQ ID NO: 263 - DNA, 16 bp, Artificial Sequence
  Template sequence
ttttccagatggcccc

SEQ ID NO: 264 - DNA, 18 bp, Artificial Sequence
  Template sequence
cgtgaggccaaagaagct

SEQ ID NO: 265 - DNA, 15 bp, Artificial Sequence
  Template sequence
gttgccaccgcccag

SEQ ID NO: 266 - DNA, 16 bp, Artificial Sequence
  Template sequence
tcgtggagttgcccac

SEQ ID NO: 267 - DNA, 17 bp, Artificial Sequence
  Template sequence
aagcccacgtccagaag

SEQ ID NO: 268 - DNA, 15 bp, Artificial Sequence
  Template sequence
tgggccccagtgtca

SEQ ID NO: 269 - DNA, 22 bp, Artificial Sequence
  Template sequence
caacaatctgggctatgagatc

SEQ ID NO: 270 - DNA, 24 bp, Artificial Sequence
  Template sequence
ccatgacaagtctctgaataagaa

SEQ ID NO: 271 - DNA, 17 bp, Artificial Sequence
  Template sequence
gcaccctctgctggtcc

SEQ ID NO: 272 - DNA, 15 bp, Artificial Sequence
  Template sequence
ggtggcgtcatgcgc

SEQ ID NO: 273 - DNA, 19 bp, Artificial Sequence
  Template sequence
ccttctcctgcaggtgacc

SEQ ID NO: 274 - DNA, 19 bp, Artificial Sequence
  Template sequence
gaagttcgtcagtcctgcc

SEQ ID NO: 275 - DNA, 18 bp, Artificial Sequence
  Template sequence
gccactcacctacgacga

SEQ ID NO: 276 - DNA, 17 bp, Artificial Sequence
  Template sequence
gagcctccaatggcatc

SEQ ID NO: 277 - DNA, 21 bp, Artificial Sequence
  Template sequence
tcttctccatccttggttaca

SEQ ID NO: 278 - DNA, 18 bp, Artificial Sequence
  Template sequence
gcccatgaacacaaggtc

SEQ ID NO: 279 - DNA, 22 bp, Artificial Sequence
  Template sequence
gcatcaactgtatcaccagctt

SEQ ID NO: 280 - DNA, 19 bp, Artificial Sequence
  Template sequence
cgccctgttctgcataacc

SEQ ID NO: 281 - DNA, 21 bp, Artificial Sequence
  Template sequence
aattgtcaccaggatcaatga

SEQ ID NO: 282 - DNA, 23 bp, Artificial Sequence
  Template sequence
tcacatctggtggagtaattttc

SEQ ID NO: 283 - DNA, 15 bp, Artificial Sequence
  Template sequence
gctgcggggagcagg

SEQ ID NO: 284 - DNA, 15 bp, Artificial Sequence
  Template sequence
cttgcagtggccgcc

SEQ ID NO: 285 - DNA, 22 bp, Artificial Sequence
  Template sequence
tggacagctgtaaatttctgct

SEQ ID NO: 286 - DNA, 19 bp, Artificial Sequence
  Template sequence
ccttctgcttggaggctgt

SEQ ID NO: 287 - DNA, 15 bp, Artificial Sequence
  Template sequence
gctgaggcctggggg

SEQ ID NO: 288 - DNA, 15 bp, Artificial Sequence
  Template sequence
gctgaggcctggggg

SEQ ID NO: 289 - DNA, 24 bp, Artificial Sequence
  Template sequence
tcactattttcttgacccctactt

SEQ ID NO: 290 - DNA, 19 bp, Artificial Sequence
  Template sequence
ccgcatccagaacattcta

SEQ ID NO: 291 - DNA, 18 bp, Artificial Sequence
  Template sequence
ggaggcccacaccaactt

SEQ ID NO: 292 - DNA, 17 bp, Artificial Sequence
  Template sequence
ttggcttccagcctgac

SEQ ID NO: 293 - DNA, 20 bp, Artificial Sequence
  Template sequence
cgcagagagagacctgcatt

SEQ ID NO: 294 - DNA, 16 bp, Artificial Sequence
  Template sequence
ggaaacaggctccccc

SEQ ID NO: 295 - DNA, 23 bp, Artificial Sequence
  Template sequence
cagtattgagatgcttctgtcca

SEQ ID NO: 296 - DNA, 18 bp, Artificial Sequence
  Template sequence
cgttgtgaagtcgtggcc

SEQ ID NO: 297 - DNA, 16 bp, Artificial Sequence
  Template sequence
gcatcgtggtgtcccg

SEQ ID NO: 298 - DNA, 17 bp, Artificial Sequence
  Template sequence
gggaggaagcggaaagt

SEQ ID NO: 299 - DNA, 15 bp, Artificial Sequence
  Template sequence
cgaggtgctggggca

SEQ ID NO: 300 - DNA, 20 bp, Artificial Sequence
  Template sequence
cacactttcttttgccacct

SEQ ID NO: 301 - DNA, 17 bp, Artificial Sequence
  Template sequence
tcctgcttctcatggcc

SEQ ID NO: 302 - DNA, 26 bp, Artificial Sequence
  Template sequence
caggtactagagtttctgcagaactt

SEQ ID NO: 303 - DNA, 19 bp, Artificial Sequence
  Template sequence
gggactgcttccattctgc

SEQ ID NO: 304 - DNA, 20 bp, Artificial Sequence
  Template sequence
gaccacaagcactcaccttc

SEQ ID NO: 305 - DNA, 19 bp, Artificial Sequence
  Template sequence
tgactgtcacctgttggga

SEQ ID NO: 306 - DNA, 19 bp, Artificial Sequence
  Template sequence
tgactgtcacctgttggga

SEQ ID NO: 307 - DNA, 17 bp, Artificial Sequence
  Template sequence
gtgggtggttgtctggc

SEQ ID NO: 308 - DNA, 15 bp, Artificial Sequence
  Template sequence
tcctgggcaggcagc

SEQ ID NO: 309 - DNA, 19 bp, Artificial Sequence
  Template sequence
cgtcagatctggtaggggg

SEQ ID NO: 310 - DNA, 20 bp, Artificial Sequence
  Template sequence
ggtcttcatatttccgggat

SEQ ID NO: 311 - DNA, 17 bp, Artificial Sequence
  Template sequence
cgtaggcatgcaggttg

SEQ ID NO: 312 - DNA, 17 bp, Artificial Sequence
  Template sequence
ggctagcacgtccccaa

SEQ ID NO: 313 - DNA, 19 bp, Artificial Sequence
  Template sequence
aagggccatcttcatcaga

SEQ ID NO: 314 - DNA, 15 bp, Artificial Sequence
  Template sequence
tcacccaggcgctgc

SEQ ID NO: 315 - DNA, 21 bp, Artificial Sequence
  Template sequence
ggaactggatgatgaagctga

SEQ ID NO: 316 - DNA, 17 bp, Artificial Sequence
  Template sequence
gcggatgctgaagagcc

SEQ ID NO: 317 - DNA, 15 bp, Artificial Sequence
  Template sequence
gccggtggaggctgc

SEQ ID NO: 318 - DNA, 17 bp, Artificial Sequence
  Template sequence
cacctcggagccgaact

SEQ ID NO: 319 - DNA, 20 bp, Artificial Sequence
  Template sequence
cacctcaggagtgtctcagc

SEQ ID NO: 320 - DNA, 15 bp, Artificial Sequence
  Template sequence
cccgacgcagggaga

SEQ ID NO: 321 - DNA, 26 bp, Artificial Sequence
  Template sequence
gaagatatttgcaaagtatgtacagc

SEQ ID NO: 322 - DNA, 16 bp, Artificial Sequence
  Template sequence
gaccatggctgggcat

SEQ ID NO: 323 - DNA, 17 bp, Artificial Sequence
  Template sequence
gcttcctcctccgctcc

SEQ ID NO: 324 - DNA, 17 bp, Artificial Sequence
  Template sequence
ccagatgtggagggcaa

SEQ ID NO: 325 - DNA, 16 bp, Artificial Sequence
  Template sequence
gcgcatgtactcgggg

SEQ ID NO: 326 - DNA, 18 bp, Artificial Sequence
  Template sequence
ctggagtctcggagtcca

SEQ ID NO: 327 - DNA, 15 bp, Artificial Sequence
  Template sequence
gcccggtggggtagc

SEQ ID NO: 328 - DNA, 17 bp, Artificial Sequence
  Template sequence
tgaacttgcgcagcatc

SEQ ID NO: 329 - DNA, 16 bp, Artificial Sequence
  Template sequence
tggctgttgcctcggt

SEQ ID NO: 330 - DNA, 16 bp, Artificial Sequence
  Template sequence
ggctggggtcctcacc

SEQ ID NO: 331 - DNA, 20 bp, Artificial Sequence
  Template sequence
catgcagcacacttagacca

SEQ ID NO: 332 - DNA, 18 bp, Artificial Sequence
  Template sequence
cggaagcccaagaagttg

SEQ ID NO: 333 - DNA, 18 bp, Artificial Sequence
  Template sequence
tctcagcagcaacatcca

SEQ ID NO: 334 - DNA, 16 bp, Artificial Sequence
  Template sequence
tccacactggctccca

SEQ ID NO: 335 - DNA, 20 bp, Artificial Sequence
  Template sequence
catgcagcacacttagacca

SEQ ID NO: 336 - DNA, 20 bp, Artificial Sequence
  Template sequence
catgcagcacacttagacca

SEQ ID NO: 337 - DNA, 27 bp, Artificial Sequence
  Template sequence
tcatgttcttacattcaagacactaaa

SEQ ID NO: 338 - DNA, 21 bp, Artificial Sequence
  Template sequence
ggggagactgttaaacaccaa

SEQ ID NO: 339 - DNA, 19 bp, Artificial Sequence
  Template sequence
accgaagtttgcaggagtc

SEQ ID NO: 340 - DNA, 19 bp, Artificial Sequence
  Template sequence
ctgctgaacagagtgagcc

SEQ ID NO: 341 - DNA, 16 bp, Artificial Sequence
  Template sequence
cagggacatgcaggcc

SEQ ID NO: 342 - DNA, 16 bp, Artificial Sequence
  Template sequence
cggcagcttcttcccc

SEQ ID NO: 343 - DNA, 16 bp, Artificial Sequence
  Template sequence
cggcagcttcttcccc

SEQ ID NO: 344 - DNA, 21 bp, Artificial Sequence
  Template sequence
gctgtagtacccagaacaacg

SEQ ID NO: 345 - DNA, 17 bp, Artificial Sequence
  Template sequence
aggtacccaaccccagg

SEQ ID NO: 346 - DNA, 15 bp, Artificial Sequence
  Template sequence
tacggtgcggccttg

SEQ ID NO: 347 - DNA, 19 bp, Artificial Sequence
  Template sequence
catggtacgtgcaccacag

SEQ ID NO: 348 - DNA, 20 bp, Artificial Sequence
  Template sequence
ctccctggctgttatcttca

SEQ ID NO: 349 - DNA, 21 bp, Artificial Sequence
  Template sequence
gatctgaggtccttggacttg

SEQ ID NO: 350 - DNA, 16 bp, Artificial Sequence
  Template sequence
cacctgctcctgctgc

SEQ ID NO: 351 - DNA, 21 bp, Artificial Sequence
  Template sequence
tcccacttttaccttccaaga

SEQ ID NO: 352 - DNA, 16 bp, Artificial Sequence
  Template sequence
tcatggctgctgtctt

SEQ ID NO: 353 - DNA, 17 bp, Artificial Sequence
  Template sequence
tcatggctgctgtgctt

SEQ ID NO: 354 - DNA, 17 bp, Artificial Sequence
  Template sequence
aggaaccaggccttggt

SEQ ID NO: 355 - DNA, 19 bp, Artificial Sequence
  Template sequence
tctgttgcctccactgaag

SEQ ID NO: 356 - DNA, 19 bp, Artificial Sequence
  Template sequence
acttggtcacgggttatcc

SEQ ID NO: 357 - DNA, 15 bp, Artificial Sequence
  Template sequence
ggactggcagggcga

SEQ ID NO: 358 - DNA, 22 bp, Artificial Sequence
  Template sequence
ccacaaagactatggagagcag

SEQ ID NO: 359 - DNA, 17 bp, Artificial Sequence
  Template sequence
ccacttccatggcggta

SEQ ID NO: 360 - DNA, 21 bp, Artificial Sequence
  Template sequence
ctactccaagtttggcaacac

SEQ ID NO: 361 - DNA, 16 bp, Artificial Sequence
  Template sequence
cgcctggcagaggacc

SEQ ID NO: 362 - DNA, 20 bp, Artificial Sequence
  Template sequence
ccactgaaatgatcacagca

SEQ ID NO: 363 - DNA, 19 bp, Artificial Sequence
  Template sequence
cttagatctggggctgtcc

SEQ ID NO: 364 - DNA, 17 bp, Artificial Sequence
  Template sequence
gcagcgggagtaatggc

SEQ ID NO: 365 - DNA, 22 bp, Artificial Sequence
  Template sequence
ggagttctggtgcgtagaattc

SEQ ID NO: 366 - DNA, 19 bp, Artificial Sequence
  Template sequence
tgtctcaccttcctgcaca

SEQ ID NO: 367 - DNA, 17 bp, Artificial Sequence
  Template sequence
aggtgatggaggcgaga

SEQ ID NO: 368 - DNA, 18 bp, Artificial Sequence
  Template sequence
ccttgaccacctcctcca

SEQ ID NO: 369 - DNA, 17 bp, Artificial Sequence
  Template sequence
ccgccaacatggtcttc

SEQ ID NO: 370 - DNA, 16 bp, Artificial Sequence
  Template sequence
ccactttggccccaaa

SEQ ID NO: 371 - DNA, 18 bp, Artificial Sequence
  Template sequence
gacgctcactgcaagtcg

SEQ ID NO: 372 - DNA, 19 bp, Artificial Sequence
  Template sequence
tcaaggagaatggtgctcc

SEQ ID NO: 373 - DNA, 16 bp, Artificial Sequence
  Template sequence
atgcagtcccaggcct

SEQ ID NO: 374 - DNA, 18 bp, Artificial Sequence
  Template sequence
agttccgcattcaacagg

SEQ ID NO: 375 - DNA, 19 bp, Artificial Sequence
  Template sequence
cgttctgcagcaccttctt

SEQ ID NO: 376 - DNA, 15 bp, Artificial Sequence
  Template sequence
gctctcctggcgcag

SEQ ID NO: 377 - DNA, 20 bp, Artificial Sequence
  Template sequence
tgaagcaccaagtctgagct

SEQ ID NO: 378 - DNA, 17 bp, Artificial Sequence
  Template sequence
ggacctccatggtgcac

SEQ ID NO: 379 - DNA, 17 bp, Artificial Sequence
  Template sequence
ggcagtagttgaggcgg

SEQ ID NO: 380 - DNA, 15 bp, Artificial Sequence
  Template sequence
cctggacccccgaag

SEQ ID NO: 381 - DNA, 20 bp, Artificial Sequence
  Template sequence
ctctgacacccctcaagttc

SEQ ID NO: 382 - DNA, 24 bp, Artificial Sequence
  Template sequence
gcagataacttcctttgtagtcca

SEQ ID NO: 383 - DNA, 19 bp, Artificial Sequence
  Template sequence
gtcaggagggagagtccag

SEQ ID NO: 384 - DNA, 22 bp, Artificial Sequence
  Template sequence
tctacctttacacgttggcttc

SEQ ID NO: 385 - DNA, 18 bp, Artificial Sequence
  Template sequence
ccttcacatgtgggcttc

SEQ ID NO: 386 - DNA, 20 bp, Artificial Sequence
  Template sequence
cagaaggaagagttctgggg

SEQ ID NO: 387 - DNA, 20 bp, Artificial Sequence
  Template sequence
cacataacgctctctggagg

SEQ ID NO: 388 - DNA, 15 bp, Artificial Sequence
  Template sequence
tccctggctcccgga

SEQ ID NO: 389 - DNA, 15 bp, Artificial Sequence
  Template sequence
caccgtctttgcgcc

SEQ ID NO: 390 - DNA, 15 bp, Artificial Sequence
  Template sequence
ccgcaggatccacca

SEQ ID NO: 391 - DNA, 19 bp, Artificial Sequence
  Template sequence
actgcactctgctccacag

SEQ ID NO: 392 - DNA, 18 bp, Artificial Sequence
  Template sequence
gctgtgctgtggagcatg

SEQ ID NO: 393 - DNA, 16 bp, Artificial Sequence
  Template sequence
agagggcccagagggt

SEQ ID NO: 394 - DNA, 20 bp, Artificial Sequence
  Template sequence
cccacccattatcagaccta

SEQ ID NO: 395 - DNA, 16 bp, Artificial Sequence
  Template sequence
gcaggttggcacggta

SEQ ID NO: 396 - DNA, 16 bp, Artificial Sequence
  Template sequence
tgagggctggacatgc

SEQ ID NO: 397 - DNA, 20 bp, Artificial Sequence
  Template sequence
acacagtccatggttccttc

SEQ ID NO: 398 - DNA, 18 bp, Artificial Sequence
  Template sequence
cactgagggagaaggcca

SEQ ID NO: 399 - DNA, 15 bp, Artificial Sequence
  Template sequence
tgcaggacgctcggc

SEQ ID NO: 400 - DNA, 19 bp, Artificial Sequence
  Template sequence
ggtatcccagagctctccc

SEQ ID NO: 401 - DNA, 20 bp, Artificial Sequence
  Template sequence
aagaaggcaaccactgtctg

SEQ ID NO: 402 - DNA, 15 bp, Artificial Sequence
  Template sequence
cgctcagctgggtcc

SEQ ID NO: 403 - DNA, 18 bp, Artificial Sequence
  Template sequence
cctccgagactgggaaca

SEQ ID NO: 404 - DNA, 16 bp, Artificial Sequence
  Template sequence
gggagccatagcgagg

SEQ ID NO: 405 - DNA, 19 bp, Artificial Sequence
  Template sequence
gggtctctatgcccaacaa

SEQ ID NO: 406 - DNA, 18 bp, Artificial Sequence
  Template sequence
agcacattcacggtcacc

SEQ ID NO: 407 - DNA, 19 bp, Artificial Sequence
  Template sequence
gacaatccctctcgtccag

SEQ ID NO: 408 - DNA, 16 bp, Artificial Sequence
  Template sequence
ccctttgggctcaacc

SEQ ID NO: 409 - DNA, 15 bp, Artificial Sequence
  Template sequence
caacgatgaggcggc

SEQ ID NO: 410 - DNA, 16 bp, Artificial Sequence
  Template sequence
ccccacttcgggttcc

SEQ ID NO: 411 - DNA, 21 bp, Artificial Sequence
  Template sequence
cgacacacagcatgaagtctg

SEQ ID NO: 412 - DNA, 24 bp, Artificial Sequence
  Template sequence
gcaaagaaaggaaagagacttacc

SEQ ID NO: 413 - DNA, 21 bp, Artificial Sequence
  Template sequence
tgcttcttttggctctgactt

SEQ ID NO: 414 - DNA, 18 bp, Artificial Sequence
  Template sequence
ccagaatgctcaccagcc

SEQ ID NO: 415 - DNA, 19 bp, Artificial Sequence
  Template sequence
tgactgtcacctgttggga

SEQ ID NO: 416 - DNA, 23 bp, Artificial Sequence
  Template sequence
cacatgatagggcttttaacaat

SEQ ID NO: 417 - DNA, 20 bp, Artificial Sequence
  Template sequence
acaatgtaattcctgccgtc

SEQ ID NO: 418 - DNA, 20 bp, Artificial Sequence
  Template sequence
ccctgcagacactacacacc

SEQ ID NO: 419 - DNA, 16 bp, Artificial Sequence
  Template sequence
ccagggcgggaagatg

SEQ ID NO: 420 - DNA, 18 bp, Artificial Sequence
  Template sequence
gtcgatgtgcaggtaggc

SEQ ID NO: 421 - DNA, 20 bp, Artificial Sequence
  Template sequence
tgttgaccttgtgttcatgg

SEQ ID NO: 422 - DNA, 18 bp, Artificial Sequence
  Template sequence
tgtggccacatcctcaat

SEQ ID NO: 423 - DNA, 17 bp, Artificial Sequence
  Template sequence
agatggcgaacccagag

SEQ ID NO: 424 - DNA, 17 bp, Artificial Sequence
  Template sequence
gcccagcccctactcac

SEQ ID NO: 425 - DNA, 21 bp, Artificial Sequence
  Template sequence
actctccttaccgtgtgtgaa

SEQ ID NO: 426 - DNA, 22 bp, Artificial Sequence
  Template sequence
gctgaactgacattagaggtga

SEQ ID NO: 427 - DNA, 15 bp, Artificial Sequence
  Template sequence
gggcgctctgggaga

SEQ ID NO: 428 - DNA, 15 bp, Artificial Sequence
  Template sequence
agggctgatgccgct

SEQ ID NO: 429 - DNA, 21 bp, Artificial Sequence
  Template sequence
atgaataggtgtgggtgtacg

SEQ ID NO: 430 - DNA, 17 bp, Artificial Sequence
  Template sequence
agctggcaagatctggg

SEQ ID NO: 431 - DNA, 19 bp, Artificial Sequence
  Template sequence
ccaggtaccacgactcctc

SEQ ID NO: 432 - DNA, 19 bp, Artificial Sequence
  Template sequence
ccaggtaccacgactcctc

SEQ ID NO: 433 - DNA, 20 bp, Artificial Sequence
  Template sequence
cccaaatacatctcccagga

SEQ ID NO: 434 - DNA, 27 bp, Artificial Sequence
  Template sequence
cataaactgtagtcactgtaggcttct

SEQ ID NO: 435 - DNA, 17 bp, Artificial Sequence
  Template sequence
ccgtgtcaggctggaag

SEQ ID NO: 436 - DNA, 15 bp, Artificial Sequence
  Template sequence
gtgttggggctgcgg

SEQ ID NO: 437 - DNA, 18 bp, Artificial Sequence
  Template sequence
gcaggactccttgcacat

SEQ ID NO: 438 - DNA, 16 bp, Artificial Sequence
  Template sequence
acggggagcttctgga

SEQ ID NO: 439 - DNA, 15 bp, Artificial Sequence
  Template sequence
ccagctggccaccga

SEQ ID NO: 440 - DNA, 15 bp, Artificial Sequence
  Template sequence
ctgagaccgcccgga

SEQ ID NO: 441 - DNA, 15 bp, Artificial Sequence
  Template sequence
ggaggcggcgcagac

SEQ ID NO: 442 - DNA, 20 bp, Artificial Sequence
  Template sequence
gaagccttgtgaatgatgct

SEQ ID NO: 443 - DNA, 18 bp, Artificial Sequence
  Template sequence
acggccatctctagcaca

SEQ ID NO: 444 - DNA, 16 bp, Artificial Sequence
  Template sequence
agggttggtggccagt

SEQ ID NO: 445 - DNA, 15 bp, Artificial Sequence
  Template sequence
ggccctctccagcgg

SEQ ID NO: 446 - DNA, 20 bp, Artificial Sequence
  Template sequence
ggctttgtcgttgcttatca

SEQ ID NO: 447 - DNA, 41 bp, Artificial Sequence
  Template sequence
agagtctataagcatcgtcgggcgacgaagcttccgaggaa

SEQ ID NO: 448 - DNA, 41 bp, Artificial Sequence
  Template sequence
tcagacaattctatacgcggtggagaggaagcagaagggct

SEQ ID NO: 449 - DNA, 38 bp, Artificial Sequence
  Template sequence
tcgtgagttgtcctgctgcagcaccctctgctggtccc

SEQ ID NO: 450 - DNA, 38 bp, Artificial Sequence
  Template sequence
gcctgtaatggtggatctcagtccccagccaggaggca

SEQ ID NO: 451 - DNA, 40 bp, Artificial Sequence
  Template sequence
gatctgtctgacgctgtatggcagccaggcaacaaccagc

SEQ ID NO: 452 - DNA, 40 bp, Artificial Sequence
  Template sequence
cgtgataatgcgtctcgtagcaggacctagaacgggcagc

SEQ ID NO: 453 - DNA, 39 bp, Artificial Sequence
  Template sequence
cattatcggacatgctcacttggagctcaagccattcaa

SEQ ID NO: 454 - DNA, 40 bp, Artificial Sequence
  Template sequence
atgatgagccgtgatgacccctgacgaatgtgatggccac

SEQ ID NO: 455 - DNA, 40 bp, Artificial Sequence
  Template sequence
tacatcgcttgcatgagtgtgagctgcagccactctacct

SEQ ID NO: 456 - DNA, 38 bp, Artificial Sequence
  Template sequence
gatctggcttcaactgtatgccggcttccatgaggcca

SEQ ID NO: 457 - DNA, 42 bp, Artificial Sequence
  Template sequence
tgcctagctttccatatcggccttgcagagcatgacatcaac

SEQ ID NO: 458 - DNA, 40 bp, Artificial Sequence
  Template sequence
tatctcgcttgctatcaacgatctaccagtccaaggaggc

SEQ ID NO: 459 - DNA, 40 bp, Artificial Sequence
  Template sequence
gcctaagctctgtcgctgattcgctctgctccaggtactt

SEQ ID NO: 460 - DNA, 39 bp, Artificial Sequence
  Template sequence
tctattgctgttcggcggcaaccctgggcctcagccagc

SEQ ID NO: 461 - DNA, 38 bp, Artificial Sequence
  Template sequence
agcagagatggacagacctcctcttcagcatccgccac

SEQ ID NO: 462 - DNA, 38 bp, Artificial Sequence
  Template sequence
gctggcggttcatgcaatcttccacctcggagccgaac

SEQ ID NO: 463 - DNA, 40 bp, Artificial Sequence
  Template sequence
tatctgcgttgctgacgtgccagttcggctccgaggtgga

SEQ ID NO: 464 - DNA, 40 bp, Artificial Sequence
  Template sequence
gatccgtatgtcgaatggctctgcagtacaaggatctgcc

SEQ ID NO: 465 - DNA, 39 bp, Artificial Sequence
  Template sequence
ccagaggtgcggtcacatatcactgaggataaggggggg

SEQ ID NO: 466 - DNA, 41 bp, Artificial Sequence
  Template sequence
gcatcttcgccagctatattggttgaccatggctgggcata

SEQ ID NO: 467 - DNA, 38 bp, Artificial Sequence
  Template sequence
cacttacggccatgctgaatcccgcgccgctggggact

SEQ ID NO: 468 - DNA, 38 bp, Artificial Sequence
  Template sequence
cactgtacgcactggagctacgtgtgcgagttcttcag

SEQ ID NO: 469 - DNA, 41 bp, Artificial Sequence
  Template sequence
gtgtgcattgagtctatgactttgaccgcttctcagaggac

SEQ ID NO: 470 - DNA, 40 bp, Artificial Sequence
  Template sequence
cgtctcatgcctgcgtatagtggtcatcgtggccatcgcc

SEQ ID NO: 471 - DNA, 43 bp, Artificial Sequence
  Template sequence
tacatcattgcgagtcatggaagagggaactgcggatgtccag

SEQ ID NO: 472 - DNA, 41 bp, Artificial Sequence
  Template sequence
atacgctctgccatacgtgagccggtaccttcttctttgcc

SEQ ID NO: 473 - DNA, 41 bp, Artificial Sequence
  Template sequence
ttgcgccatttggacatgctacctctggttttcagctcacg

SEQ ID NO: 474 - DNA, 41 bp, Artificial Sequence
  Template sequence
gcctgatattcattcacagcacatcacacccgggtacccac

SEQ ID NO: 475 - DNA, 40 bp, Artificial Sequence
  Template sequence
ctgtcgtctagtctctgaggcatgcagcacacttagacca

SEQ ID NO: 476 - DNA, 38 bp, Artificial Sequence
  Template sequence
tttcgtgctttggagacagcaatggtcgggatgctggc

SEQ ID NO: 477 - DNA, 40 bp, Artificial Sequence
  Template sequence
tgccgtgttggtgcttcacactctctggacttcacagaac

SEQ ID NO: 478 - DNA, 39 bp, Artificial Sequence
  Template sequence
tcgtccactttagcatgatgaagactggctgctccctga

SEQ ID NO: 479 - DNA, 40 bp, Artificial Sequence
  Template sequence
tacatacttgcagtgctggcactttgagctggaaagcagc

SEQ ID NO: 480 - DNA, 39 bp, Artificial Sequence
  Template sequence
cgtcgtgctgcgtgactataggaaagcagccgtttctcc

SEQ ID NO: 481 - DNA, 46 bp, Artificial Sequence
  Template sequence
tgagagtctgttcttaggcccatttttgcattgccttcggtttgta

SEQ ID NO: 482 - DNA, 40 bp, Artificial Sequence
  Template sequence
tacataattgccatgacgggttcaatctggctgtgctatt

SEQ ID NO: 483 - DNA, 40 bp, Artificial Sequence
  Template sequence
gagaatgctgtatagtgtcctttctgggaaccttggcccc

SEQ ID NO: 484 - DNA, 40 bp, Artificial Sequence
  Template sequence
cgtctcgctggtcactaatggtgtaactcgaccctgcacc

SEQ ID NO: 485 - DNA, 40 bp, Artificial Sequence
  Template sequence
gatctctgtgaagttagtgccctctgccctctgcacctcc

SEQ ID NO: 486 - DNA, 40 bp, Artificial Sequence
  Template sequence
tataaagattgcggtcaggcccctcagctataaatagggc

SEQ ID NO: 487 - DNA, 40 bp, Artificial Sequence
  Template sequence
ccagtcggtgtagcagcaattagggcctcgtgacccggcc

SEQ ID NO: 488 - DNA, 38 bp, Artificial Sequence
  Template sequence
gtgtgctcttctcgctgcaagccaggggaagaagctgc

SEQ ID NO: 489 - DNA, 38 bp, Artificial Sequence
  Template sequence
ataccggctgctacacagtgaacggcgccaagatgccc

SEQ ID NO: 490 - DNA, 41 bp, Artificial Sequence
  Template sequence
caaatagtgtgcgaggatctgctatttaaaggtacgcgccg

SEQ ID NO: 491 - DNA, 42 bp, Artificial Sequence
  Template sequence
tgagacattgtgcaaatcggacatgtgccctctcgctggctt

SEQ ID NO: 492 - DNA, 42 bp, Artificial Sequence
  Template sequence
gatagcagttcactacctgggtctgtgttctctttgcagtac

SEQ ID NO: 493 - DNA, 41 bp, Artificial Sequence
  Template sequence
ggcatcactggttacgtctgatctgaggtccttggacttga

SEQ ID NO: 494 - DNA, 40 bp, Artificial Sequence
  Template sequence
gtctgacttgagttacatgggagcaacagcaggaacagca

SEQ ID NO: 495 - DNA, 42 bp, Artificial Sequence
  Template sequence
ggtcttcctatatgtgcgcgtcctggtggtggttctgtcgat

SEQ ID NO: 496 - DNA, 42 bp, Artificial Sequence
  Template sequence
tgagaagttgtgaagatccctaaccatctgtgctttctcccc

SEQ ID NO: 497 - DNA, 38 bp, Artificial Sequence
  Template sequence
gccaggcgttcagatgcaatcccagggacttgtacagc

SEQ ID NO: 498 - DNA, 38 bp, Artificial Sequence
  Template sequence
gctggtcgtggtccaatcattgagggacctgggtccgc

SEQ ID NO: 499 - DNA, 41 bp, Artificial Sequence
  Template sequence
gaccatgctggcttacctgtaagctacaccaactgtccagc

SEQ ID NO: 500 - DNA, 43 bp, Artificial Sequence
  Template sequence
tggcatcgtttcacctgctggacgaatagctatgggaagaaca

SEQ ID NO: 501 - DNA, 40 bp, Artificial Sequence
  Template sequence
tatcattctgtggtcggcgcccaggaggagaactacctgc

SEQ ID NO: 502 - DNA, 38 bp, Artificial Sequence
  Template sequence
gtggatcttgatgtaatgcctagcccgggcggagctgg

SEQ ID NO: 503 - DNA, 39 bp, Artificial Sequence
  Template sequence
gccgtcaatgggtgctcaatatctgccgtcccatgctgg

SEQ ID NO: 504 - DNA, 41 bp, Artificial Sequence
  Template sequence
gccagtcattccacgtatatagagtggtgtgggcactttga

SEQ ID NO: 505 - DNA, 38 bp, Artificial Sequence
  Template sequence
gccagccatgtgtcgaatgagggcacggtacctgggct

SEQ ID NO: 506 - DNA, 45 bp, Artificial Sequence
  Template sequence
atctcagagtggcatcggatagaagcattgtgtgccatctatatt

SEQ ID NO: 507 - DNA, 43 bp, Artificial Sequence
  Template sequence
gtctgcaattatcggctgtgtctggtatgtgttttccctgcag

SEQ ID NO: 508 - DNA, 41 bp, Artificial Sequence
  Template sequence
ggtctgcattcgctgatatgagcgatacgctgacccgacac

SEQ ID NO: 509 - DNA, 40 bp, Artificial Sequence
  Template sequence
gcgaattgaagccagttgcaagaagaacgccatggcccac

SEQ ID NO: 510 - DNA, 41 bp, Artificial Sequence
  Template sequence
ccatcgaatcgtctatcagtactttgtgctgacggctgctc

SEQ ID NO: 511 - DNA, 41 bp, Artificial Sequence
  Template sequence
ggtctcaattaggcttcatgtactccgctctggccaccttg

SEQ ID NO: 512 - DNA, 41 bp, Artificial Sequence
  Template sequence
gccggtcatgtgctctgatatcaccacactggccaaggaca

SEQ ID NO: 513 - DNA, 39 bp, Artificial Sequence
  Template sequence
gcgtgatattccatgatctgaggttctggaatcccggag

SEQ ID NO: 514 - DNA, 43 bp, Artificial Sequence
  Template sequence
gctggtgatggctcttcatatggaggactacctggatatcaag

SEQ ID NO: 515 - DNA, 42 bp, Artificial Sequence
  Template sequence
cgaacatctgtcacaatgcgctcggttagcgaccaattgtca

SEQ ID NO: 516 - DNA, 44 bp, Artificial Sequence
  Template sequence
gactctagtgtcgtctgatctctttggtgaaaccatggtagaag

SEQ ID NO: 517 - DNA, 41 bp, Artificial Sequence
  Template sequence
tcagatgttgtaatcgtgcgcaaggtgtggaaggagcactt

SEQ ID NO: 518 - DNA, 41 bp, Artificial Sequence
  Template sequence
gcgtcggcttcatgcgatattacaccagcatcgtggcggag

SEQ ID NO: 519 - DNA, 40 bp, Artificial Sequence
  Template sequence
atgcacgatcctctacattgggacttctcccaggccctga

SEQ ID NO: 520 - DNA, 41 bp, Artificial Sequence
  Template sequence
cttacccatgattagcgcagggaaccccgacgtgcagcaga

SEQ ID NO: 521 - DNA, 41 bp, Artificial Sequence
  Template sequence
gccgatggtgcgtctactatgtctgtttttggagcgagtgg

SEQ ID NO: 522 - DNA, 40 bp, Artificial Sequence
  Template sequence
tggcaggttgtgactctctcaaccggagttgccctcagac

SEQ ID NO: 523 - DNA, 42 bp, Artificial Sequence
  Template sequence
tatgattattgagtgcggccctgcgactccaagatgaaaccc

SEQ ID NO: 524 - DNA, 40 bp, Artificial Sequence
  Template sequence
tcagatcgtcttgctgtcgaacccagaggaagccggcctt

SEQ ID NO: 525 - DNA, 41 bp, Artificial Sequence
  Template sequence
tttgagatttgtcgagagccactgacccctattccctgctt

SEQ ID NO: 526 - DNA, 44 bp, Artificial Sequence
  Template sequence
gcctgctgtggctgtatatcagataacttcctttgtagtccatc

SEQ ID NO: 527 - DNA, 37 bp, Artificial Sequence
  Template sequence
gatcactgtggtccctgtctgtagctggactttctgc

SEQ ID NO: 528 - DNA, 41 bp, Artificial Sequence
  Template sequence
tatgagtgttgcgctatgcctcatctgggaattgggacaac

SEQ ID NO: 529 - DNA, 40 bp, Artificial Sequence
  Template sequence
gcgtcgctgtcgtgtactatccacaggggagtgggacaac

SEQ ID NO: 530 - DNA, 39 bp, Artificial Sequence
  Template sequence
atacgggatgatgagcatactgctgcaggccccagatga

SEQ ID NO: 531 - DNA, 45 bp, Artificial Sequence
  Template sequence
tacatgacttgccctgctgtttcatgatcccaagctgaaaggcaa

SEQ ID NO: 532 - DNA, 39 bp, Artificial Sequence
  Template sequence
acgatgagcagggatcactaacaggtgcagcacgcagcc

SEQ ID NO: 533 - DNA, 40 bp, Artificial Sequence
  Template sequence
atctgagagctagtcggcatccaccctctctcagaaggtc

SEQ ID NO: 534 - DNA, 42 bp, Artificial Sequence
  Template sequence
ggtgactattcggctgctctaccagcaatgacaacatgggct

SEQ ID NO: 535 - DNA, 43 bp, Artificial Sequence
  Template sequence
tagctgtgttgacatctggcacagaaacaccacagcactaatt

SEQ ID NO: 536 - DNA, 40 bp, Artificial Sequence
  Template sequence
tgcttagttgtgagtcgccagagcagagtgcagtgtgcct

SEQ ID NO: 537 - DNA, 42 bp, Artificial Sequence
  Template sequence
ctcacgactgggctgatgattccatccctccaggcaccctca

SEQ ID NO: 538 - DNA, 44 bp, Artificial Sequence
  Template sequence
tggcacagtttcctgctggtggctccacctgtcatttctcttgt

SEQ ID NO: 539 - DNA, 41 bp, Artificial Sequence
  Template sequence
gctgggtgtgatcctctctacaagagaatggccactggtca

SEQ ID NO: 540 - DNA, 39 bp, Artificial Sequence
  Template sequence
ggtgacagtgtattatctgcatcacttcctactccaggg

SEQ ID NO: 541 - DNA, 41 bp, Artificial Sequence
  Template sequence
gatctgttcaaagtgatggcgtcagcacctggaagccccca

SEQ ID NO: 542 - DNA, 41 bp, Artificial Sequence
  Template sequence
tatcttattctcgacgcggctcccacgagagcatcatctgc

SEQ ID NO: 543 - DNA, 38 bp, Artificial Sequence
  Template sequence
cctgtctaccatgcagtaatcggcgcaacatccgtgca

SEQ ID NO: 544 - DNA, 38 bp, Artificial Sequence
  Template sequence
tatatgcagtggtgttcgcctatcccagagctctccct

SEQ ID NO: 545 - DNA, 41 bp, Artificial Sequence
  Template sequence
gacgcgggtgctcatcatatctgcgcaggtgaacggcagcg

SEQ ID NO: 546 - DNA, 40 bp, Artificial Sequence
  Template sequence
gctgggcatgtgtactactctgatggagcgatgttgggct

SEQ ID NO: 547 - DNA, 42 bp, Artificial Sequence
  Template sequence
ctgtcaatgcgtctgctctagaccgtggtctgttccctggac

SEQ ID NO: 548 - DNA, 41 bp, Artificial Sequence
  Template sequence
gtctcgtcttcgtgagtgcagctactcagagttgcaacctc

SEQ ID NO: 549 - DNA, 39 bp, Artificial Sequence
  Template sequence
acgcacactgataactatgcacctcctgtgaccagccca

SEQ ID NO: 550 - DNA, 38 bp, Artificial Sequence
  Template sequence
gtgctgggttcgcattcatcgcacattcacggtcacct

SEQ ID NO: 551 - DNA, 41 bp, Artificial Sequence
  Template sequence
ccaataggtgctcacgtcatgtgtttttccagatggccccc

SEQ ID NO: 552 - DNA, 41 bp, Artificial Sequence
  Template sequence
ttggctcatttgcatggcgccacgtgaggccaaagaagctg

SEQ ID NO: 553 - DNA, 41 bp, Artificial Sequence
  Template sequence
tgctcgcttgtgatcgactgttgccaccgcccagggcccgg

SEQ ID NO: 554 - DNA, 38 bp, Artificial Sequence
  Template sequence
cctgtcgcgcctgatagaatgtcgtggagttgcccacc

SEQ ID NO: 555 - DNA, 44 bp, Artificial Sequence
  Template sequence
acgcaatatcggccatcgtggcaaaaaagcccacgtccagaagg

SEQ ID NO: 556 - DNA, 41 bp, Artificial Sequence
  Template sequence
ctgtgccctgctctgatgattactatgggccccagtgtcag

SEQ ID NO: 557 - DNA, 41 bp, Artificial Sequence
  Template sequence
gtgcctgttgacatatagtgacaatctgggctatgagatca

SEQ ID NO: 558 - DNA, 47 bp, Artificial Sequence
  Template sequence
cctgtagtgcagtctcctgacgcatgacaagtctctgaataagaagt

SEQ ID NO: 559 - DNA, 40 bp, Artificial Sequence
  Template sequence
cactcactggcacggtatagtgttgggatgcctcctggct

SEQ ID NO: 560 - DNA, 40 bp, Artificial Sequence
  Template sequence
ggaatgtctgccgtgccataatggtggcgtcatgcgcgcc

SEQ ID NO: 561 - DNA, 38 bp, Artificial Sequence
  Template sequence
ctgtgagtgatgtacgctccttctcctgcaggtgacca

SEQ ID NO: 562 - DNA, 43 bp, Artificial Sequence
  Template sequence
gcgtgcggttcatctgcattctggaagttcgtcagtcctgcct

SEQ ID NO: 563 - DNA, 41 bp, Artificial Sequence
  Template sequence
cggctgggtagcatcatctaaagccactcacctacgacgac

SEQ ID NO: 564 - DNA, 38 bp, Artificial Sequence
  Template sequence
gcatgaagttccataatcgcgagcctccaatggcatca

SEQ ID NO: 565 - DNA, 45 bp, Artificial Sequence
  Template sequence
cagtgacatgccgctcagtacatcttctccatccttggttacatg

SEQ ID NO: 566 - DNA, 38 bp, Artificial Sequence
  Template sequence
cggcaatatgatgataggtccccatgaacacaaggtca

SEQ ID NO: 567 - DNA, 45 bp, Artificial Sequence
  Template sequence
cctggtatgacatggagcctcagcatcaactgtatcaccagcttc

SEQ ID NO: 568 - DNA, 40 bp, Artificial Sequence
  Template sequence
ccaacgatgctactgagtcacgccctgttctgcataacca

SEQ ID NO: 569 - DNA, 41 bp, Artificial Sequence
  Template sequence
cattgcacccactgagatggattgtcaccaggatcaatgac

SEQ ID NO: 570 - DNA, 41 bp, Artificial Sequence
  Template sequence
cacggatctgccgctagaatcatctggtggagtaattttcc

SEQ ID NO: 571 - DNA, 37 bp, Artificial Sequence
  Template sequence
cgaacacatgcggctggataagctgcggggagcaggg

SEQ ID NO: 572 - DNA, 38 bp, Artificial Sequence
  Template sequence
agatagagtcgatgccagctttgcagtggccgccgccg

SEQ ID NO: 573 - DNA, 45 bp, Artificial Sequence
  Template sequence
tgcctcattgtgactcatggacagctgtaaatttctgctggacaa

SEQ ID NO: 574 - DNA, 42 bp, Artificial Sequence
  Template sequence
tgtgagcttgttactacggctgccttctgcttggaggctgtg

SEQ ID NO: 575 - DNA, 38 bp, Artificial Sequence
  Template sequence
tgtgaatatgtgtgtgccactgaggcctggggggcacc

SEQ ID NO: 576 - DNA, 38 bp, Artificial Sequence
  Template sequence
gtagactatttaggctgtgctcctcctcatcggggccc

SEQ ID NO: 577 - DNA, 41 bp, Artificial Sequence
  Template sequence
gatcgcagttcagagcgcatattttcttgacccctacttac

SEQ ID NO: 578 - DNA, 45 bp, Artificial Sequence
  Template sequence
cagtctcgtggatagcactcgttctccgcatccagaacattctat

SEQ ID NO: 579 - DNA, 38 bp, Artificial Sequence
  Template sequence
gactgggattacatgctatggaggcccacaccaacttt

SEQ ID NO: 580 - DNA, 39 bp, Artificial Sequence
  Template sequence
cactccgatggcgagatgaatttggcttccagcctgaca

SEQ ID NO: 581 - DNA, 39 bp, Artificial Sequence
  Template sequence
gcaccgtctgtcgatctatacagagagagacctgcattg

SEQ ID NO: 582 - DNA, 40 bp, Artificial Sequence
  Template sequence
agccaagtgcaggcgtacatcctggaaacaggctccccca

SEQ ID NO: 583 - DNA, 44 bp, Artificial Sequence
  Template sequence
tcctctcgttggatgtgagccagtattgagatgcttctgtccaa

SEQ ID NO: 584 - DNA, 38 bp, Artificial Sequence
  Template sequence
cagtgacgtgagtgccatctgttgtgaagtcgtggccc

SEQ ID NO: 585 - DNA, 38 bp, Artificial Sequence
  Template sequence
ctcagcagttagcagcgcatcgcatcgtggtgtcccgc

SEQ ID NO: 586 - DNA, 40 bp, Artificial Sequence
  Template sequence
cttatggcgctgtcggctatcagggaggaagcggaaagtc

SEQ ID NO: 587 - DNA, 38 bp, Artificial Sequence
  Template sequence
gatatgcgttacgtgagtctcggccatctctagcacag

SEQ ID NO: 588 - DNA, 44 bp, Artificial Sequence
  Template sequence
caacaactgcgcgacgatgaaacacactttcttttgccacctac

SEQ ID NO: 589 - DNA, 41 bp, Artificial Sequence
  Template sequence
ttgtgcattgttggacgcccctttcctgcttctcatggcca

SEQ ID NO: 590 - DNA, 46 bp, Artificial Sequence
  Template sequence
agcagtaatgacagcgtgcaaggtactagagtttctgcagaactta

(19) United States
(12) Patent Application Publication
(10) Pub. No.: US 2005/0074787 A1
(43) Pub. Date: Apr. 7, 2005

(54) Universal arrays

(75) Inventors: Jian-Bing Fan, San Diego, CA (US); Joel N. Hirschhorn, Newton, MA (US); Xiaohua Huang, Mountain View, CA (US); Paul Kaplan, Campbell, CA (US); Eric S. Lander, Cambridge, MA (US); David J. Lockhart, Del Mar, CA (US); Thomas Ryder, Los Gatos, CA (US); Pamela Sklar, Brookline, MA (US)

Correspondence Address: Lisa M. Treannie, Esq.;Ropes & Gray LLP, One International Place, Boston, MA, 02110-2624, US

(73) Assignees: Whitehead Institute for Biomedical Research, Cambridge, MA (US); The General Hospital Corporation, Boston, MA (US); Affymetrix, Inc, Santa Clara, CA (US)

(21) Appl. No.: 10/730,771

(22) Filed: Dec. 8, 2003

Related U.S. Application Data
(62) Division of application No. 09/536,841, filed on Mar. 27, 2000.
(60) Provisional application No. 60/126,473, filed on Mar. 26, 1999.
(60) Provisional application No. 60/140,359, filed on Jun. 23, 1999.

(51) Int. Cl. C12Q 1/68

(52) U.S. Cl. 435/6

(57) ABSTRACT

An array of oligonucleotides on a solid substrate is disclosed, which can be used for multiple purposes. Methods and reagents are provided for performing genotyping to determine the identity or ratio of allelic forms of a gene in a sample. A single base extension primer is coupled to a sequence identity code. During the primer extension reaction a distinctive label is incorporated which identifies the allelic form present in the sample. This permits multiple simultaneous analyses to be performed easily and efficiently.

### RELATED APPLICATIONS

This application is a divisional of U.S. application Ser. No. 09/536,841, filed Mar. 27, 2000, which claims the benefit of U.S. Provisional Application Ser. Nos. 60/126,473, filed Mar. 26, 1999, and 60/140,359, filed Jun. 23, 1999, the entire teachings of which are incorporated herein by reference.

### BACKGROUND OF THE INVENTION

Obtaining genotype information on thousands of polymorphic markers in a highly parallel fashion is becoming an increasingly important task in mapping disease loci, in identifying quantitative trait loci, in diagnosing tumor loss of heterozygosity, and in performing linkage studies. A currently available method for simultaneously obtaining large numbers of polymorphic marker genotypes involves hybridization to allele specific probes on high density oligonucleotide arrays. In order to practice the method, redundant sets of hybridization probes, typically twenty or more, are used to score each marker. A high degree of redundancy is required, however, to reduce the noise and achieve an acceptable level of accuracy. Even this level of redundancy is often insufficient to unambiguously score heterozygotes or to quantitatively determine allele frequency in a population. Thus, there is a need in the art for more reliable and better quantitative methods to identify genotypes at polymorphic markers.

### SUMMARY OF THE INVENTION

An array of oligonucleotide tags attached to a solid substrate is disclosed, along with locus-specific tagged oligonucleotides. The array and the locus-specific tagged oligonucleotides are particularly useful in genotyping using single base extension reactions. When used together, the array and the locus-specific tagged oligonucleotides serve as a “universal chip” system for use in genotyping, wherein by using different sets of locus-specific tagged oligonucleotides the system can be tailored to any desired genotyping application. For example, it is an object of the present invention to provide a method to aid in determining a ratio of alleles at a polymorphic locus. It is another object of the invention to provide a set of primers for use in determining a ratio of nucleotides present at a polymorphic locus.

Thus, in one embodiment the invention relates to an array comprising one or more oligonucleotide tags fixed to a solid substrate, wherein each oligonucleotide tag comprises a unique known arbitrary nucleotide sequence of sufficient length to hybridize to a locus-specific tagged oligonucleotide, wherein the locus-specific tagged oligonucleotide has at its first end nucleotide sequence which hybridizes to, e.g., is complementary to, the arbitrary sequence of the oligonucleotide tag, and wherein the locus-specific tagged oligonucleotide has at a second end nucleotide sequence complementary to target polynucleotide sequence in a sample.

In one embodiment, the invention relates to a kit comprising an array comprising one or more oligonucleotide tags fixed to a solid substrate, wherein each oligonucleotide tag comprises a unique known arbitrary nucleotide sequence of sufficient length to hybridize to a locus-specific tagged oligonucleotide, and one or more locus-specific tagged oligonucleotides, wherein each locus-specific tagged oligonucleotide has at its first (5′) end nucleotide sequence which hybridizes to, e.g., is complementary to, the arbitrary sequence of a corresponding oligonucleotide tag on the array, and has at it's second (3′) end nucleotide sequence complementary to target polynucleotide sequence in a sample.

The invention further relates to a method of genotyping a nucleic acid sample at one or more loci, comprising the steps of obtaining a nucleic acid sample to be tested; combining the nucleic acid sample with one or more locus-specific tagged oligonucleotides under conditions suitable for hybridization of the nucleic acid sample to one or more locus-specific tagged oligonucleotides, wherein each locus-specific tagged oligonucleotide comprises a nucleotide sequence capable of hybridizing to a complementary sequence in an oligonucleotide tag and a nucleotide sequence complementary to the nucleotide sequence 5′ of a nucleotide to be queried in the sample, thereby creating an amplification product-locus-specific tagged oligonucleotide complex; subjecting the complex to a single base extension reaction, wherein the reaction results in the addition of a labeled ddNTP to the locus-specific tagged oligonucleotide, and wherein each type of ddNTP has a label that can be distinguished from the label of the other three types of ddNTPs; contacting the complex with an oligonucleotide array comprising one or more oligonucleotide tags fixed to a solid substrate under suitable hybridization conditions, wherein each oligonucleotide tag comprises a unique arbitrary sequence complementary and of sufficient length to hybridize to a complementary sequence in a locus-specific tagged oligonucleotide, whereby the complex hybridizes to a specific oligonucleotide tag on the array; and assaying the array to determine the labeled ddNTPs present in the complex hybridized to one or more oligonucleotide tags, thereby determining the genotype of the queried nucleotide in the sample. In one embodiment the nucleic acid sample to be tested is amplified.

In one embodiment a method is provided to aid in determining a ratio of alleles at a polymorphic locus in a sample. A pair of primers is used to amplify a region of a nucleic acid in a sample. In one embodiment, the region comprises a polymorphic locus, and an amplified nucleic acid product is formed which comprises the polymorphic locus. The amplified nucleic acid product is used as a template in a single base extension reaction with an extension primer, forming a labeled extension primer. The extension primer (also called a locus-specific tagged oligonucleotide herein) comprises a 3′ portion and a 5′ portion. The 3′ portion is complementary to the amplified nucleic acid product and terminates one nucleotide 5′ to the polymorphic locus. The 5′ portion is not complementary to the amplified nucleic acid product. A labeled dideoxynucleotide which is complementary to the polymorphic locus is coupled to the 3′ end of the extension primer. Each type of dideoxynucleotide present in the reaction bears a distinct label. The 5′. portion of the extension primer is hybridized to one or more probes (also called oligonucleotide tags herein) which are immobilized to known locations on a solid support. The probes comprise a nucleotide sequence which is complementary to the 5′ portion of the extension primer.

Also provided by the present invention is a set of primers for use in determining a ratio of nucleotides present at a polymorphic locus. The set includes a pair of amplification primers and an extension primer. The pair of primers prime synthesis of a region of double stranded nucleic acid which comprises a polymorphic locus. The extension primer comprises a 3′ portion which is complementary to a portion of the region of double stranded nucleic acid and a 5′ portion which is not complementary to the region of double stranded nucleic acid. The extension primer terminates one nucleotide 5′ to the polymorphic locus. Examples of primers according to the invention are shown in Table 1.

Another embodiment of the invention provides a method to aid in determining a ratio of alleles at a polymorphic locus in a sample. Any nucleic acid molecule, including genomic DNA, which comprises one or more polymorphic locus is used as a template in a single base extension reaction with an extension primer, forming a labeled extension primer. The extension primer comprises a 3′ portion and a 5′ portion. The 3′ portion is complementary to the nucleic acid molecule and terminates one nucleotide 5′ to the polymorphic locus. The 5′ portion is not complementary to the nucleic acid molecule. A labeled dideoxynucleotide which is complementary to the polymorphic locus is coupled to the 3′ end of the extension primer. Each type of dideoxynucleotide present in the reaction bears a distinct label. The 5′ portion of the extension primer is hybridized to one or more probes which are immobilized to known locations on a solid support.

These and other embodiments of the invention which are described in more detail below provide the art with methods and tools for rapidly and easily determining genotypes of individuals and allele frequencies in populations.

### BRIEF DESCRIPTION OF THE DRAWINGS

FIG. 1 is a diagram of the universal array: The solid substrate (e.g., a glass slide) is depicted on the left, and different oligonucleotide tags (“A”, “B”, “C”, etc.) are shown attached to the solid substrate. The nucleotide sequence on the right-hand end of each oligonucleotide tag (“Tag A”, “Tag B”, “Tag C”) is arbitrary unique sequence; that is, it is designed and synthesized to be unique to each oligonucleotide tag.

FIG. 2 is a diagram depicting a locus-specific tagged oligonucleotide. The nucleotide sequence at the left-hand end is complementary to the arbitrary sequence of one of the oligonucleotide tags depicted in FIG. 1. The nucleotide sequence at the right-hand end is complementary to the amplification product of a known polymorphic locus (e.g., a single nucleotide polymorphism (SNP)). Therefore, locus-specific tagged oligonucleotide “A” comprises a nucleotide sequence complementary to the arbitrary sequence of the “Tag A” oligonucleotide tag depicted in FIG. 1, and also comprises sequence complementary to SNP “A”.

FIG. 3 is a diagram showing the hybridization of the locus-specific tagged oligonucleotide to the amplification product. The locus-specific sequence (right hand end) of the oligonucleotide is designed so that it terminates one nucleotide immediately before (5′ of) the nucleotide to be genotyped (shown in box).

FIG. 4 is a diagram depicting the labeling of the locus-specific tagged oligonucleotide-amplification primer complex via single base extension. During the reaction, a single labeled ddNTP complementary to the queried nucleotide is enzymatically added to the 3′ end of the locus-specific tagged oligonucleotide. The nucleotide is shown in the box.

FIG. 5 is a diagram depicting the hybridization of the complex of the amplification product and the locus-specific tagged oligonucleotide to the oligonucleotide tags on the array. The solid substrate to which the oligonucleotide tags of the array are bound is shown on the left, with the individual addresses labeled as “A”, “B”, etc. Each oligonucleotide tag is shown at its address. The locus-specific tagged oligonucleotide is shown hybridized to the oligonucleotide tag, and the amplification product is in turn bound to the locus-specific tagged oligonucleotide. The locus-specific tagged oligonucleotide is bound to a labeled (▪,•, etc.) nucleotide as a result of single base extension. Although a single complex is shown at each address, in reality, many such oligonucleotide tags are located at each address; that is, the substrate surface at address “A” has many copies of oligonucleotide tag “A” attached to it, etc.

FIG. 6 is a diagram depicting the hybridization as in FIG. 5, but the sample at address “B” is heterozygous for the queried nucleotide.

FIG. 7 is a schematic showing the combined use of amplification, single base extension of a tagged primer, and hybridization to a tag array.

FIG. 8 shows a quantitative measurement of allele frequency. Template-T (5′-TGCTGAATATTCAGATTCTCTAGTGCTACCTGAAAGATCCTG-3′; SEQ ID NO: 1) and Template-G (5′-TGCTGAATATTCAGATTCTCGAGTGCTACCTGAAAGATCCTG-3′; SEQ ID NO: 2) were mixed at different ratios (6 nM/60 nM, 6 nM/18 nM, 6 nM/6 nM, 18 nM/6 nM, 60 nM/6 nM, 180 nM/6 nM). Six SBE primers
(5′-CACCATGCTCACAATGAATGCAGGATCTTTCAGGTAGCACT-3′ (SEQ ID NO: 3);
5′ -GATAATTCTCTGATAGGCCGCAGGATCTTTCAGGTAGCACT-3′ (SEQ ID NO: 4);
5′-GACTACGATGTGATCCGTGTCAGGATCTTTCAGGTAGCACT-3′ (SEQ ID NO: 5);
5′-GAACGCAGTTATCAGACTCTCAGGATCTTTCAGGTAGCACT-3′ (SEQ ID NO: 6);
5′-CGAGGACATGGAGTCACATCCAGGATCTTTCAGGTAGCACT-3′ (SEQ ID NO: 7); and
5′-GCTAGGCATTCCTCCAGTGTCAGGATCTTTCAGGTAGCACT-3′ (SEQ ID NO: 8)) were separately added to six SBE reactions which contain the mixed templates of different ratios. The SBE primers were extended in the presence of biotin-labeled ddATP and fluorescein-labeled ddCTP (see Examples) and pooled and hybridized to the tag array. The intensity ratio of the two colors (the y-axis) were plotted against the ratio of the mixed two templates (the x-axis).

FIG. 9 shows a clustering analysis of the tag array hybridization results in 44 individuals at marker GMP-140.25.

### DETAILED DESCRIPTION OF THE INVENTION

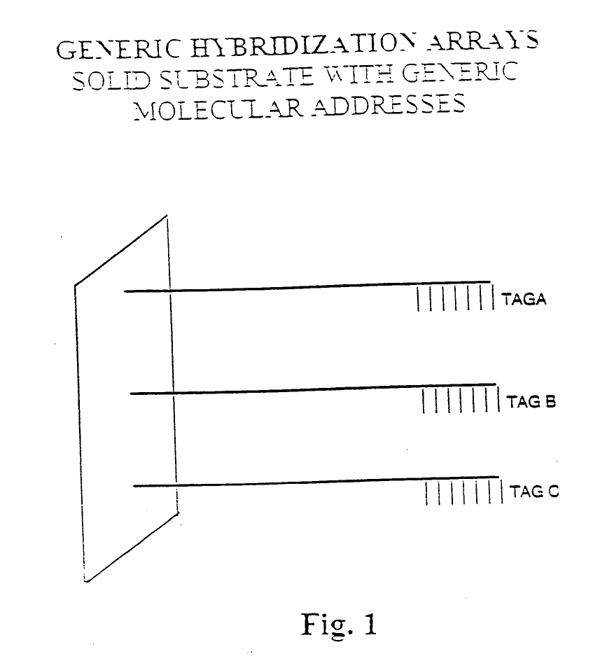

The invention features a generic or universal genotyping array, consisting of oligonucleotide tags attached to a solid substrate (FIG. 1). Each address in the array (e.g., “A”, “B”, “C”, etc.) has an oligonucleotide tag associated with it. The oligonucleotide tag at a given address is attached to the solid substrate, and comprises a unique arbitrary nucleotide sequence. That is, the nucleotide sequence is unique for the oligonucleotide tag at each address, i.e., the nucleotide sequence for “tag A” is different from the nucleotide sequence for all other tags in the array. The nucleotide sequence for each tag is arbitrary in that it can be any sequence, provided that it is different from the nucleotide sequence for every other tag in the array. Preferably the oligonucleotide tag is from about 20 to about 50 nucleotides in length. It may also be desirable to design the nucleotide sequence of the oligonucleotide tag such that it does not facilitate an undesirable interaction, e.g., with the target nucleic acid molecule (amplified product).

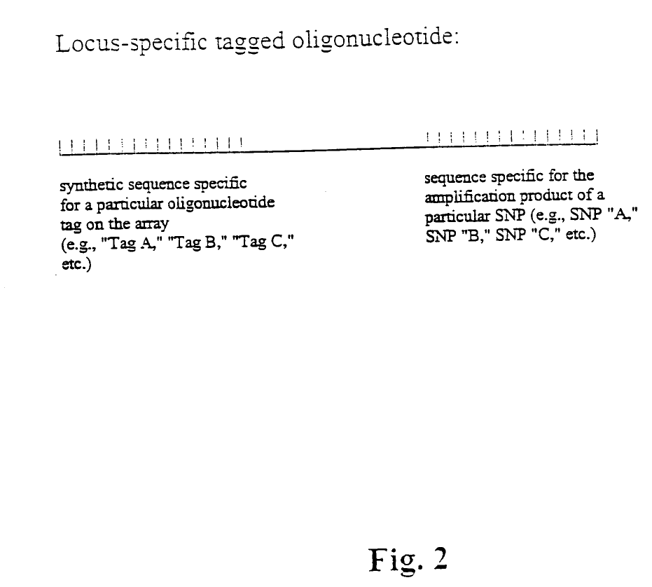

The oligonucleotide array is used in conjunction with locus-specific tagged oligonucleotides. Each oligonucleotide tag in the array corresponds to a locus-specific tagged oligonucleotide. One end (the 5′ end) of the locus-specific tagged oligonucleotide comprises a nucleotide sequence complementary to the unique arbitrary sequence of its corresponding oligonucleotide tag (FIG. 2). Preferably, this sequence is from about 20 to about 30 nucleotides long. The other end (the 3′ end) of the locus-specific tagged oligonucleotide is complementary to a target nucleic acid molecule comprising a nucleotide to be queried, e.g., a polymorphic nucleotide. Preferably, the 3′ end of locus-specific tagged oligonucleotide is synthesized such that when hybridized to the target nucleic acid molecule the locus-specific tagged oligonucleotide terminates one nucleotide 5′ to the nucleotide to be queried. The portion of the locus-specific tagged oligonucleotide which hybridizes to the target nucleic acid molecule is preferably from about 15 to about 30 nucleotides long. For example, the 5′ end of locus-specific tagged oligonucleotide “A” would be complementary to the unique arbitrary sequence at the end of the oligonucleotide tag “A” which is bound to address “A” in the array. The 3′ end of locus-specific tagged oligonucleotide “A” would be complementary to the polynucleotide sequence 5′ of the nucleotide to be queried in target “A”.

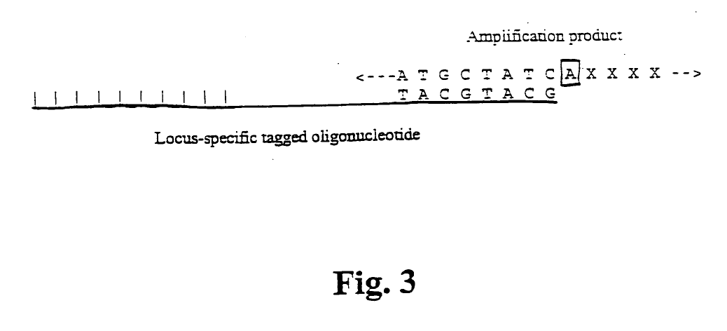

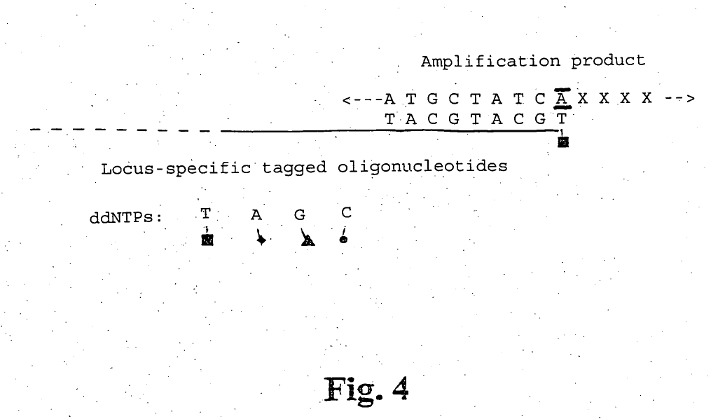

To genotype a nucleic acid sample from an individual at locus “A”, amplification primers specific for the region containing locus “A” are used to amplify the nucleic acid molecules in the sample. Locus-specific tagged oligonucleotides complementary to the nucleotide sequence 5′ of locus “A” are combined with the amplification products under conditions suitable for hybridization (FIG. 3). The hybridization complex is subjected to single base extension. The four types of ddNTPs in the reaction mixture have different labels (e.g., four different fluorescent tags, e.g., the ddATPs would have an attached fluorophore that fluoresced at a first wavelength, the ddCTPs would have an attached fluorophore that fluoresced at a second wavelength, the ddGTPs would have an attached fluorophore that fluoresced at a third wavelength, and the ddTTPs would have an attached fluorophore that fluoresced at a fourth wavelength). During the single base extension reaction, a single ddNTP is attached (FIG. 4), resulting in the formation of a complex composed of the locus-specific tagged oligonucleotide extended with the labeled ddNTP and the amplification product.

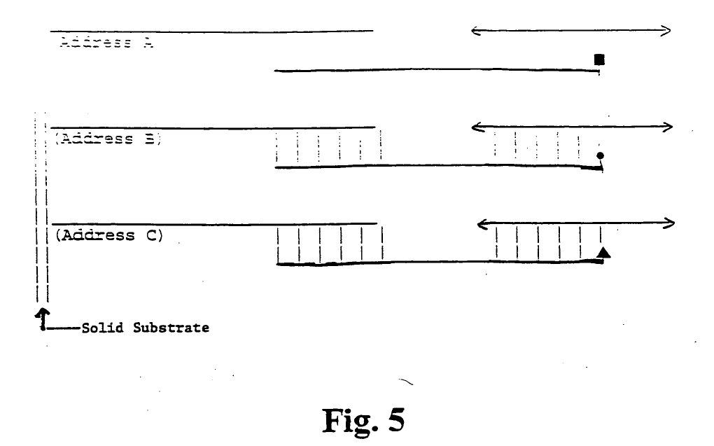

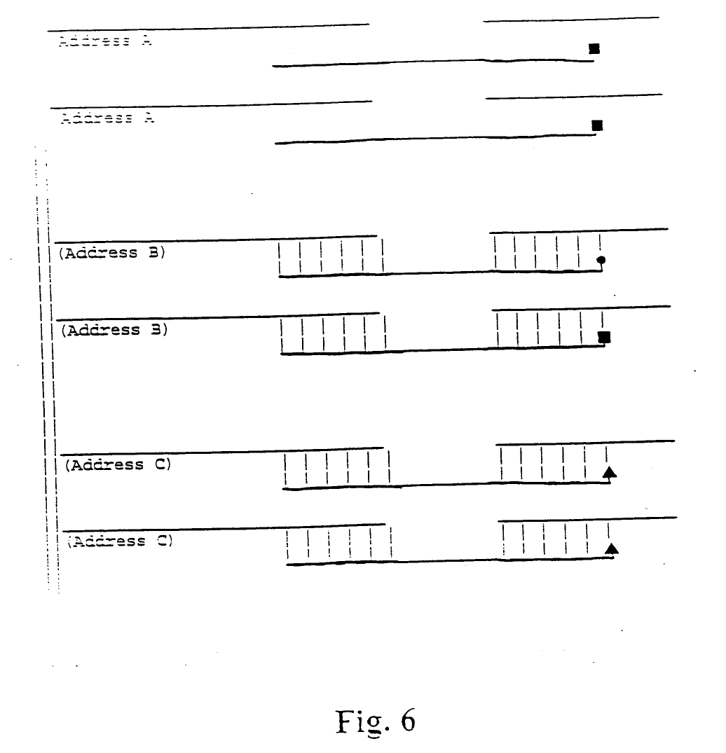

After the single base extension reaction, the complex of the labeled (extended) locus-specific tagged oligonucleotide and the amplification product is hybridized to the array (FIG. 5). The oligonucleotide tag “A” at address “A” selectively hybridizes to its corresponding locus-specific tagged oligonucleotide (now extended with a labeled ddNTP), the oligonucleotide tag “B” at address “B” selectively hybridizes to its corresponding locus-specific tagged oligonucleotide (now extended with a labeled ddNTP), etc. The array is assayed to determine which label(s) is (are)present at which address on the array. For instance, if address “A” fluoresced at the same wavelength as the label on the ddATP, then the amplification product clearly contained a “T” at the queried nucleotide (because the single base extension reaction attaches the ddNTP complementary to the queried nucleotide). Fluorescence at a wavelength which is the same as the ddCTP label would indicate that the genotype was a “G”, etc. Detection of two peaks within the wavelength emitted would indicate that different nucleotides were present at the queried position in the sample, e.g., that the individual was heterozygous at that locus.

An advantage of the array and method described herein is that many addresses can be assayed simultaneously, producing genotyping data for many different genetic loci, e.g., SNPs. By utilizing a predefined set of locus-specific tagged oligonucleotides, e.g., a set specific for assaying a set of genetic diseases, a single array can be utilized for a particular purpose, and by utilizing a different set of locus-specific tagged oligonucleotides which correspond to the same tags on the array, the same array can be utilized for a different purpose. The universal chip serves as the repository of a set of addresses to which the locus-specific tagged oligonucleotides (along with the labeled, genotyped SNPs) hybridize in a planned, predetermined manner. The array and set(s) of locus-specific tagged oligonucleotides can therefore be used as components in kits for the purposes of sequencing and genotyping. Sets of locus-specific tagged oligonucleotides can therefore be used in combination with arrays as described herein for use in forensics, identification of individuals, and disease diagnosis/prognosis.

The present invention provides a convenient and accurate way of determining the genotype of an individual at a polymorphic locus or the frequency of alleles in a population. One embodiment of the method involves three steps: (1) amplification of a polymorphic locus, (2) primer extension of a sequence-tagged primer with distinct labels for different polynucleotides at the polymorphic locus, and (3) hybridization to a tag array. The amount of each distinct label can be determined at known positions of the tag array. Each tag represents a distinct polymorphic locus and each distinct label represents a distinct allelic form at the polymorphic locus. The method permits the simultaneous determination of a genotype at multiple loci, as well as the determination of allele frequencies in a population. Another embodiment employs just steps 2 and 3.

Advantages of the disclosed method include that just one generic tag array can be used to genotype any genetic marker, i.e., no specific customized genotyping chip is needed. In addition, the pre-selected probe sequences synthesized on the tag chip guarantee good hybridization results between the probe and the tag. Moreover, the two color or multiple color approach used in this assay provides accurate measurement of the allele frequency in the samples tested. This means very reliable genotype results can be obtained not only for individual samples, but also for pooled samples.

A pair of primers or a single primer can be used to amplify a region of a nucleic acid in a sample. The sample may be from a single individual or may be from a population of individuals. The region which is amplified includes a polymorphic locus. The step of amplification is not specific for a particular allele. However, the amplification is designed to specifically amplify regions of double stranded or single stranded nucleic acids which contain polymorphic loci.

The amplification step may be carried out using any technique known in the art. One preferred technique is polymerase chain reaction (PCR) in which DNA is amplified logarithmically. As is known in the art, each primer of a pair of amplification primers hybridizes to, and is preferrably complementary to, opposite strands of an allele. It is preferred that the primers hybridize to a double stranded nucleic acid in locations which are not more than 2 kb apart, and preferably which are much closer together, such as not more than 1 kb, 0.5 kb, 0.2 kb, 0.1 kb, 0.01 kb or 0.001 kb apart. A suitable DNA polymerase can be used as is known in the art. Thermostable polymerases are particularly convenient for thermal cycling of rounds of primer hybridization, polymerization, and melting. Amplification of single stranded nucleic acids can also be employed.

After the amplification it is desirable to remove and/or degrade any excess primers and nucleotides. This can be done by washing and/or enzymatic degradation, using such enzymes as endonuclease I and alkaline phosphatase, for example. Other techniques, such as chromatography, magnetic beads, and avidin- or streptavidin-conjugated beads, as are known in the art for accomplishing the removal can also be used. It is not necessary to remove or destroy one of two strands of an amplified DNA product.

The primer extension step of the method is the one which provides allele-specificity to the method. The primer is designed to terminate one nucleotide 5′ to the polymorphic locus. The primer is hybridized to the denatured amplified double stranded DNA. When the primer is extended by a single base using dideoxynucleotides and a DNA polymerase, the dideoxynucleotide which is complementary to the nucleotide at the polymorphic locus is added. Again, any DNA-dependent DNA polymerase can be used. These include, but are not limited to, E. coli DNA polymerase I, Klenow fragment of polymerase I, T4 DNA polymerase, T7 DNA polymerase, T. aquaticus DNA polymerase. This reaction is preferably performed at the T_{M} of the primer with the template to enhance product formation.

One configuration for carrying out the primer extension step utilizes two different primers which each hybridize to opposite strands of an amplified double stranded DNA. Each primer terminates one nucleotide 5′ to the polymorphic locus. The primer extension reaction may be more robust with one strand as a template than the other. In addition, the information obtained from the second strand should confirm the information obtained from the first strand.

An alternative method for primer extension involves use of reverse transcriptase and one or two primers which hybridize 3′ to the polymorphic locus. This method may be desirable in cases where “forward” direction primer extension is less robust than is desirable.

Each different dideoxynucleotide present in the single base extension reaction is uniquely labeled. The unique label can be detected and its amount will be proportional to the amount of the particular allele containing the corresponding deoxynucleotide in the sample. If the sample is from a single individual, the nucleotide bases present at the polymorphic locus can be determined. If the sample is from a population of individuals the allele frequency in the population can be determined.

The ability to perform the method of the present invention in a multiplex manner for a number of different polymorphic loci simultaneously is due to the sequence tags which are present on the extension primers at their 5′ ends. The sequence tags permit the method operator to ultimately sort the products of multiplex amplification and multiplex primer base extension to different locations on an array. Each sequence tag on an extension primer is used only for a single polymorphic locus. Thus the products of primer extension reactions can be separately analyzed because they can be hybridized to distinct known locations on an array.

The sequence tags are typically totally unrelated to the sequences of the polymorphic alleles which are being analyzed. The sequence tags are chosen for their favorable hybridization characteristics. The tags are typically selected so that they have similar hybridization characteristics and minimal cross-hybridization to other tag sequences. Each sequence tag is attached to a specific gene or genetic marker, and then serves as a label for that particular gene or genetic marker. A generic tag array, corresponding to the pre-selected tag sequences is fabricated and used to detect the presence or absence or ratio of specific allelic forms in a test sample. See application Ser. No. 08/626,285 filed Apr. 4, 1996, and EP application no. 97302313.8 which are expressly incorporated by reference herein.

The labels which are used can be any which are known in the art. These include radiolabels, fluorescent labels, enzyme labels, epitope labels, and high affinity binding partner labels. Examples include isotopically labeled nucleotides, fluorescein-labeled nucleotides, biotin-labeled nucleotides, digoxin labeled nucleotides. A different label is assigned to each base dideoxynucleotide in the single base extension reaction. Two, three, or four different labels can be used in the reaction. The different labels can be all of the same type, e.g., enzyme labels, or they can be mixed types.

Hybridization of the 5′ portion of the extension primers (the tag sequences) to one or more probes which are immobilized to known locations on a solid support is also contemplated. Hybridization can be performed under standard conditions known in the art for obtaining robust signals at high specificity. Standard washing conditions can also be employed. Detection of hybridization of the extension primers can be done using standard means, depending on the type of labels used. For example, fluorescence can be detected and quantified using optical detection means. Radiolabels can be detected using autoradiography or scintillation counting. Enzyme labels can be detected using enzymatic reactions and assaying for the final product of the enzyme reaction. Antigenic labels can be used using immunological detection means. Affinity binding partners such as strepavidin or avidin and biotin can also be used as a label.

The reactions of the present invention can be performed in a single or multiplex format. For example, the amplification step can be performed using up to 20, 30, 40, 50, 75, 100, 150, 200, 250, or 300 different primer pairs to amplify a corresponding number of polymorphic markers. These can be pooled for the single base extension reaction, if desired. Pooling for the hybridization step is desirable so that thousands of hybridizations can be done simultaneously.

In an alternative embodiment the amplification step can be omitted. Thus, if sufficient DNA is available, the single base extension reaction can be performed directly on genomic DNA. In another particular embodiment, amplification of the entire genome can be performed using random primers.

Sets of primers according to the present invention comprise an amplification pair and an extension primer. These are used together in a method for determining a ratio of nucleotides present at a polymorphic locus. These may be packaged in a single container, preferably a divided container or package. The pair of primers amplify a region of double stranded DNA which comprises a polymorphic locus. The extension primer has two portions, a 3′ portion which is complementary to a portion of the region of double stranded DNA which contains the polymorphic locus and a 5′ portion which is not complementary to the region of double stranded DNA. The 5′ region is the tag sequence which is complementary to the tag array which is used to sort and analyze the products of the single base extension reaction. The 3′ end of the single base extension primer terminates one nucleotide 5′ to the polymorphic locus.

Kits according to the present invention may contain one or more sets of primers as described above. The kit may also contain a solid support comprising at least one probe which is attached to the solid support. The one or more probes are complementary to the 5′ portion of the extension primer, i.e., to the tag sequences. Solid supports, according to the present invention include beads, microtiter plates, and arrays.

Hybridizing Nucleic Acids to Arrays of Allele-Specific Probes

“Hybridization” refers to the formation of a bimolecular complex of two different nucleic acids through complementary base pairing. Complementary base pairing occurs through non-covalent bonding, usually hydrogen bonding, of bases that specifically recognize other bases, as in the bonding of complementary bases in double-stranded DNA. In this invention, hybridization is carried out between a target nucleic acid, which is prepared from the nucleic acid sample by allele-specific amplification, and at least two probes which have been immobilized on a substrate to form an array.

One of skill in the art will appreciate that an enormous number of array designs are suitable for the practice of this invention. An array will typically include a number of probes that specifically hybridize to the sequences of interest (tags). In addition, it is preferred that the array include one or more control probes. In one embodiment, the array is a high density array. A high density array is an array used to hybridize with a target nucleic acid sample to detect the presence of a large number of allelic markers, preferably more than 10, more preferably more than 100, and most preferably more than 1000 allelic markers.

High density arrays are suitable for quantifying small variations in the frequency of an allelic marker in the presence of a large population of heterogeneous nucleic acids. Such high density arrays can be fabricated either by de novo synthesis on a substrate or by spotting or transporting nucleic acid sequences onto specific locations of a substrate. Both of these methods produce nucleic acids which are immobilized on the array at particular locations. Nucleic acids can be purified and/or isolated from biological materials, such as a bacterial plasmid containing a cloned segment of a sequence of interest. Suitable nucleic acids can also be produced by amplification of templates or by synthesis. As a nonlimiting illustration, polymerase chain reaction and/or in vitro transcription, are suitable nucleic acid amplification methods.

The term “target nucleic acid” refers to a nucleic acid (either synthetic or derived from a biological sample or nucleic acid sample), to which the probe is designed to specifically hybridize. In this invention, such target nucleic acids are the same as the sequence tags. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term “target nucleic acid” can refer to the specific subsequence of a larger nucleic acid to which the probe is directed or to the overall sequence (e.g., gene or mRNA) whose presence it is desired to detect. The difference in usage will be apparent from context.

As used herein a “probe” is defined as a nucleic acid, capable of binding to a target nucleic acid of complementary sequence through one or more types of chemical bonds, usually through complementary base pairing, usually through hydrogen bond formation. As used herein, a probe can include natural (i.e. A, G, U, C, or T) or modified bases (e.g., 7-deazaguanosine, inosine, etc.). A probe can also include an oligonucleotide. An oligonucleotide is a single-stranded nucleic acid of 2 to n bases, where n can be any integer less than 1000. Nucleic acids can be cloned or synthesized using any technique known in the art. They can also include non-naturally occurring nucleotide analogs, such as those which are modified to improve hybridization, and peptide nucleic acids. In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Probe Design

An array includes “test probes”, also termed “oligonucleotide tags” herein. Test probes can be oligonucleotides that range from about 5 to about 45 or 5 to about 500 nucleotides, more preferably from about 10 to about 40 nucleotides and most preferably from about 15 to about 40 nucleotides in length. In other particularly preferred embodiments the probes are 20 to 25 nucleotides in length. In another embodiment, test probes are double or single stranded DNA sequences. DNA sequences can be isolated or cloned from natural sources or amplified from natural sources using natural nucleic acids as templates. However, in situ synthesis of probes on the arrays is preferred. The probes have sequences complementary to particular subsequences of the genes whose allelic markers they are designed to detect. Thus, the test probes are capable of specifically hybridizing to the target nucleic acid they are designed to detect.

The term “perfect match probe” refers to a probe which has a sequence designed to be perfectly complementary to a particular target sequence. The probe is typically perfectly complementary to a portion (subsequence) of the target sequence. The perfect match probe can be a “test probe,” a “normalization control probe,” an expression level control probe and the like. A perfect match control or perfect match probe is, however, distinguished from a “mismatch control” or “mismatch probe” or “mismatch control probe.”

In addition to test probes that bind the target nucleic acid(s) of interest, the high density array can contain a number of control probes. The control probes fall into two categories: normalization controls and mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency, and other factors that may cause the signal of a perfect hybridization to vary between arrays. In a preferred embodiment, signals (e.g., fluorescence intensity) read from all other probes in the array are divided by the signal (e.g., fluorescence intensity) from the control probes, thereby normalizing the measurements.

Virtually any probe can serve as a normalization control. However, it is recognized that hybridization efficiency varies with base composition and probe length. Preferred normalization probes are selected to reflect the average length of the other probes present in the array; however, they can be selected to cover a range of lengths. The normalization control(s) can also be selected to reflect the (average) base composition of the other probes in the array; however in a preferred embodiment, only one or a few normalization probes are used and they are selected such that they hybridize well (i.e. no secondary structure) and do not match any target-specific probes.

Mismatch controls can also be provided for the probes to the target alleles or for normalization controls. The terms “mismatch control” or “mismatch probe” or “mismatch control probe” refer to a probe whose sequence is deliberately selected not to be perfectly complementary to a particular target sequence. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C, or a T for an A) at any of positions 6 through 14 (the central mismatch).

For each mismatch control in a high-density array there typically exists a corresponding perfect match probe that is perfectly complementary to the same particular target sequence. The mismatch may comprise one or more bases. While the mismatch(s) may be located anywhere in the mismatch probe, terminal mismatches are less desirable, as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

Mismatch probes provide a control for non-specific binding or cross-hybridization to a nucleic acid in the sample other than the target to which the probe is directed. Mismatch probes thus indicate whether or not a hybridization is specific. For example, if the target is present, the perfect match probes should be consistently brighter than the mismatch probes. The difference in intensity between the perfect match and the mismatch probe (I_{(PM)}−I_{(MM)}) provides a good measure of the concentration of the hybridized material.

The array can also include sample preparation/amplification control probes. These are probes that are complementary to subsequences of control genes selected because they do not normally occur in the nucleic acids of the particular biological sample being assayed. Suitable sample preparation/amplification control probes include, for example, probes to bacterial genes (e.g., Bio B) where the sample in question is from a eukaryote.

In a preferred embodiment, oligonucleotide probes in the high density array are selected to bind specifically to the nucleic acid target to which they are directed with minimal non-specific binding or cross-hybridization under the particular hybridization conditions utilized. Because the high density arrays of this invention can contain in excess of 100,000 or even 1,000,000 different probes, it is possible to provide every probe of a characteristic length that binds to a particular nucleic acid sequence.

Forming High Density Arrays

High density arrays are particularly useful for monitoring the presence of allelic markers. The fabrication and application of high density arrays in gene expression monitoring have been disclosed previously in, for example, WO 97/10365, WO 92/10588, U.S. application Ser. No. 08/772,376 filed Dec. 23, 1996; Ser. No. 08/529,115 filed on Sep. 15, 1995; Ser. No. 08/168,904 filed Dec. 15, 1993; Ser. No. 07/624,114 filed on Dec. 6, 1990, Ser. No. 07/362,901 filed Jun. 7, 1990, and in U.S. Pat. No. 5,677,195, all incorporated herein for all purposes by reference. In some embodiments using high density arrays, high density oligonucleotide arrays are synthesized using methods such as the Very Large Scale Immobilized Polymer Synthesis (VLSIPS) disclosed in U.S. Pat. No. 5,445,934 incorporated herein for all purposes by reference. Each oligonucleotide occupies a known location on a substrate. A nucleic acid target sample is hybridized with a high density array of oligonucleotides and then the amount of target nucleic acids hybridized to each probe in the array is quantified.

Synthesized oligonucleotide arrays are particularly preferred for this invention. Oligonucleotide arrays have numerous advantages over other methods, such as efficiency of production, reduced intra- and inter array variability, increased information content, and high signal-to-noise ratio.

Preferred high density arrays comprise greater than about 100, preferably greater than about 1000, more preferably greater than about 16,000, and most preferably greater than 65,000 or 250,000 or even greater than about 1,000,000 different oligonucleotide probes, preferably in less than 1 cm² of surface area. The oligonucleotide probes range from about 5 to about 50 or about 500 nucleotides, more preferably from about 10 to about 40 nucleotides, and most preferably from about 15 to about 40 nucleotides in length.

Methods of forming high density arrays of oligonucleotides, peptides and other polymer sequences with a minimal number of synthetic steps are known. The oligonucleotide analogue array can be synthesized on a solid substrate by a variety of methods, including, but not limited to, light-directed chemical coupling and mechanically directed coupling.. See Pirrung et al., U.S. Pat. No. 5,143,854 (see also PCT Application No. WO 90/15070) and Fodor et al., PCT Publication Nos. WO 92/10092 and WO 93/09668 and U.S. Ser. No. 07/980,523, which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. application Ser. Nos. 07/796,243 and 07/980,523.

The development of VLSIPS™ technology as described in the above-noted U.S. Pat. No. 5,143,854 and PCT patent publication Nos. WO 90/15070 and 92/10092, is considered pioneering technology in the fields of combinatorial synthesis and screening of combinatorial libraries. More recently, patent application Ser. No. 08/082,937, filed Jun. 25, 1993, describes methods for making arrays of oligonucleotide probes that can be used to check or determine a partial or complete sequence of a target nucleic acid and to detect the presence of a nucleic acid containing a specific oligonucleotide sequence.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In the event that an oligonucleotide analogue with a polyamide backbone is used in the VLSIPS™ procedure, it is generally inappropriate to use phosphoramidite chemistry to perform the synthetic steps, since the monomers do not attach to one another via a phosphate linkage. Instead, peptide synthetic methods are substituted. See, e.g., Pirrung et al. U.S. Pat. No. 5,143,854.

Peptide nucleic acids are commercially available from, e.g., Biosearch, Inc. (Bedford, Mass.) which comprise a polyamide backbone and the bases found in naturally occurring nucleosides. Peptide nucleic acids are capable of binding to nucleic acids with high specificity, and are considered “oligonucleotide analogues” for purposes of this disclosure.

Additional methods which can be used to generate an array of oligonucleotides on a single substrate are described in co-pending Application Ser. Nos. 07/980,523, filed Nov. 20, 1992, and 07/796,243, filed Nov. 22, 1991 and in PCT Publication No. WO 93/09668. In the methods disclosed in these applications, reagents are delivered to the substrate by either (1) flowing within a channel defined on predefined regions or (2) “spotting” on predefined regions or (3) through the use of photoresist. However, other approaches, as well as combinations of spotting and flowing, can be employed. In each instance, certain activated regions of the substrate are mechanically separated from other regions when the monomer solutions are delivered to the various reaction sites.

A typical “flow channel” method applied to the compounds and libraries of the present invention can generally be described as follows. Diverse polymer sequences are synthesized at selected regions of a substrate or solid support by forming flow channels on a surface of the substrate through which appropriate reagents flow or in which appropriate reagents are placed. For example, assume a monomer “A” is to be bound to the substrate in a first group of selected regions. If necessary, all or part of the surface of the substrate in all or a part of the selected regions is activated for binding by, for example, flowing appropriate reagents through all or some of the channels, or by washing the entire substrate with appropriate reagents. After placement of a channel block on the surface of the substrate, a reagent having the monomer A flows through or is placed in all or some of the channel(s). The channels provide fluid contact to the first selected regions, thereby binding the monomer A on the substrate directly or indirectly (via a spacer) in the first selected regions.

Thereafter, a monomer “B” is coupled to second selected regions, some of which can be included among the first selected regions. The second selected regions will be in fluid contact with a second flow channel(s) through translation, rotation, or replacement of the channel block on the surface of the substrate; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second selected locations. In this particular, example, the resulting sequences bound to the substrate at this stage of processing will be, for example, A, B, and AB. The process is repeated to form a vast array of sequences of desired length at known locations on the substrate.

After the substrate is activated, monomer A can be flowed through some of the channels, monomer B can be flowed through other channels, a monomer C can be flowed through still other channels, etc. In this manner, many or all of the reaction regions are reacted with a monomer before the channel block must be moved or the substrate must be washed and/or reactivated. By making use of many or all of the available reaction regions simultaneously, the number of washing and activation steps can be minimized.

One of skill in the art will recognize that there are alternative methods of forming channels or otherwise protecting a portion of the surface of the substrate. For example, according to some embodiments, a protective coating such as a hydrophilic or hydrophobic coating (depending upon the nature of the solvent) is utilized over portions of the substrate to be protected, sometimes in combination with materials that facilitate wetting by the reactant solution in other regions. In this manner, the flowing solutions are further prevented from passing outside of their designated flow paths.

High density nucleic acid arrays can be fabricated by depositing presynthezied or natural nucleic acids in predetermined positions. Synthesized or natural nucleic acids are deposited on specific locations of a substrate by light directed targeting and oligonucleotide directed targeting. Nucleic acids can also be directed to specific locations in much the same manner as the flow channel methods. For example, a nucleic acid A can be delivered to and coupled with a first group of reaction regions which have been appropriately activated. Thereafter, a nucleic acid B can be delivered to and reacted with a second group of activated reaction regions. Nucleic acids are deposited in selected regions. Another embodiment uses a dispenser that moves from region to region to deposit nucleic acids in specific spots. Typical dispensers include a micropipette or capillary pin to deliver nucleic acid to the substrate and a robotic system to control the position of the micropipette with respect to the substrate. In other embodiments, the dispenser includes a series of tubes, a manifold, an array of pipettes or capillary pins, or the like so that various reagents can be delivered to the reaction regions simultaneously.

Hybridization Conditions

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH.

The Tₘ is the temperature, under defined ionic strength, pH, and nucleic acid concentration, at which 50% of the probes complementary to the target sequence hybridize to the target sequence at equilibrium. As the target sequences are generally present in excess, at Tₘ, 50% of the probes are occupied at equilibrium). Typically, stringent conditions will be those in which the salt concentration is at least about 0.01 to 1.0 M concentration of a Na or other salt at pH 7.0 to 8.3 and the temperature is at least about 30° C. for short probes (e.g., 10 to 50 nucleotides). Stringent conditions can also be achieved with the addition of destabilizing agents such as formamide.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) of DNA or RNA. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches.

One of skill in the art will appreciate that hybridization conditions can be selected to provide any degree of stringency. In a preferred embodiment, hybridization is performed at low stringency, in this case in 6×SSPE-T at 37° C. (0.005% Triton X-100), to ensure hybridization, and then subsequent washes are performed at higher stringency (e.g., 1×SSPE-T at 37° C.) to eliminate mismatched hybrid duplexes. Successive washes can be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity can be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array can be washed at successively higher stringency solutions and read between each wash. Analysis of the data sets thus produced will reveal a wash stringency above which the hybridization pattern is not appreciably altered and which provides adequate signal for the particular oligonucleotide probes of interest.

The stability of duplexes formed between RNAs or DNAs are generally in the order of RNA:RNA>RNA:DNA>DNA:DNA, in solution. Long probes have better duplex stability with a target, but poorer mismatch discrimination than shorter probes (mismatch discrimination refers to the measured hybridization signal ratio between a perfect match probe and a single base mismatch probe). Shorter probes (e.g., 8-mers) discriminate mismatches very well, but the overall duplex stability is low.

Altering the thermal stability (Tₘ) of the duplex formed between the target and the probe using, e.g., known oligonucleotide analogues allows for optimization of duplex stability and mismatch discrimination. One useful aspect of altering the Tm arises from the fact that adenine-thymine (A-T) duplexes have a lower Tₘ than guanine-cytosine (G-C) duplexes, due in part to the fact that the A-T duplexes have two hydrogen bonds per base-pair, while the G-C duplexes have three hydrogen bonds per base pair. In heterogeneous oligonucleotide arrays in which there is a non-uniform distribution of bases, it is not generally possible to optimize hybridization for each oligonucleotide probe simultaneously. Thus, in some embodiments, it is desirable to selectively destabilize G-C duplexes and/or to increase the stability of A-T duplexes. This can be accomplished, e.g., by substituting guanine residues in the probes of an array which form G-C duplexes with hypoxanthine, or by substituting adenine residues in probes which form A-T duplexes with 2,6 diaminopurine or by using tetramethyl ammonium chloride (TMACl) in place of NaCl.

Altered duplex stability conferred by using oligonucleotide analogue probes can be ascertained by following, e.g., fluorescence signal intensity of oligonucleotide analogue arrays hybridized with a target oligonucleotide over time. The data allow optimization of specific hybridization conditions at, e.g., room temperature.

Another way of verifying altered duplex stability is by following the signal intensity generated upon hybridization with time. Previous experiments using DNA targets and DNA chips have shown that signal intensity increases with time, and that the more stable duplexes generate higher signal intensities faster than less stable duplexes. The signals reach a plateau or “saturate” after a certain amount of time due to all of the binding sites becoming occupied. These data allow for optimization of hybridization, and determination of the best conditions at a specified temperature.

Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)).

Signal Detection

The hybridized nucleic acids can be detected by detecting one or more labels attached to the target nucleic acids. The labels can be incorporated by any of a number of means well known to those of skill in the art. However, in a preferred embodiment, the label is incorporated by labeling the primers prior to the amplification step in the preparation of the target nucleic acids. Thus, for example, polymerase chain reaction with labeled primers will provide a labeled amplification product.

Detectable labels suitable for use in the present invention include any composition detectable by spectroscopic, photochemical, biochemical, immunochemical, electrical, optical, or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels can be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label. One method uses colloidal gold label that can be detected by measuring scattered light.

Means of detecting labeled target nucleic acids hybridized to the probes of the array are known to those of skill in the art. Thus, for example, where a calorimetric label is used, simple visualization of the label is sufficient. Where a radioactive labeled probe is used, detection of the radiation (e.g. with photographic film or a solid state detector) is sufficient.

Detection of target nucleic acids which are labeled with a fluorescent label (i.e., a “color tag”) can be accomplished with fluorescence microscopy. The hybridized array can be excited with a light source at the excitation wavelength of the particular fluorescent label and the resulting fluorescence at the emission wavelength is detected. The excitation light source can be a laser appropriate for the excitation of the fluorescent label.

The confocal microscope can be automated with a computer-controlled stage to automatically scan the entire high density array, i.e., to sequentially examine individual probes or adjacent groups of probes in a systematic manner until all probes have been examined. Similarly, the microscope can be equipped with a phototransducer (e.g., a photomultiplier, a solid state array, a CCD camera, etc.) attached to an automated data acquisition system to automatically record the fluorescence signal produced by hybridization to each oligonucleotide probe on the array. Such automated systems are described at length in U.S. Pat. No. 5,143,854, PCT Application 20 92/10092, and copending U.S. application Ser. No. 08/195,889, filed on Feb. 10, 1994. Use of laser illumination in conjunction with automated confocal microscopy for signal detection permits detection at a resolution of better than about 100 μm, more preferably better than about 50 μm, and most preferably better than about 25 μm.

Two different fluorescent labels can be used in order to distinguish two alleles at each marker examined. In such a case, the array can be scanned two times. During the first scan, the excitation and emission wavelengths are set as required to detect one of the two fluorescent labels. For the second scan, the excitation and emission wavelengths are set as required to detect the second fluorescent label. When the results from both scans are compared, the genotype identification or allele frequency can be determined.

Quantification and Determination of Genotypes

The term “quantifying” when used in the context of quantifying hybridization of a nucleic acid sequence or subsequence can refer to absolute or to relative quantification. Absolute quantification can be accomplished by inclusion of known concentration(s) of one or more target nucleic acids (e.g., control nucleic acids such as Bio B, or known amounts the target nucleic acids themselves) and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, the frequency of an allele. Relative quantification can also be used to merely detect the presence or absence of an allele in the target nucleic acids. In one embodiment, for example, the presence or absence of the two alleles of a marker can be determined by comparing the quantities of the first and second color tag at the known locations in the array, i.e., on the solid support, which correspond to the allele-specific probes for the two alleles.

A preferred quantifying method is to use a confocal microscope and fluorescent labels. The GeneChip® system (Affymetrix, Santa Clara, Calif.) is particularly suitable for quantifying the hybridization; however, it will be apparent to those of skill in the art that any similar system or other effectively equivalent detection method can also be used.

Methods for evaluating the hybridization results vary with the nature of the specific probes used, as well as the controls. Simple quantification of the fluorescence intensity for each probe can be determined. This can be accomplished simply by measuring signal strength at each location (representing a different probe) on the high density array (e.g., where the label is a fluorescent label, detection of the florescence intensity produced by a fixed excitation illumination at each location on the array).

One of skill in the art, however, will appreciate that hybridization signals will vary in strength with efficiency of hybridization, the amount of label on the sample nucleic acid and the amount of the particular nucleic acid in the sample. Typically nucleic acids present at very low levels (e.g., <1 pM) will show a very weak signal. At some low level of concentration, the signal becomes virtually indistinguishable from background. In evaluating the hybridization data, a threshold intensity value can be selected below which a signal is counted as being essentially indistinguishable from background.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target allele, for the lowest 5% to 10% of the probes for each allele. However, where the probes to a particular allele hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample, such as bacterial genes where the sample is mammalian nucleic acids). Background can also be calculated as the average signal intensity produced by regions of the array that lack any probes at all. In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred embodiment, the hybridization is performed in the presence of about 0.5 mg/ml DNA (e.g., herring sperm DNA). The use of blocking agents in hybridization is well known to those of skill in the art (see, e.g., Chapter 8 in P. Tijssen, supra).

The high density array can include mismatch controls. In a preferred embodiment, there is a mismatch control having a central mismatch for every probe in the array, except the normalization controls. It is expected that after washing in stringent conditions, where a perfect match would be expected to hybridize to the probe, but not to the mismatch, the signal from the mismatch controls should only reflect non-specific binding or the presence in the sample of a nucleic acid that hybridizes with the mismatch. Where both the probe in question and its corresponding mismatch control show high signals, or the mismatch shows a higher signal than its corresponding test probe, there is a problem with the hybridization and the signal from those probes is ignored. For a given marker, the difference in hybridization signal intensity (Iₐₗₗₑₗₑ₁−Iₐₗₗₑₗₑ₂) between an allele-specific probe (perfect match probe) for a first allele and the corresponding probe for a second allele (or other mismatch control probe) is a measure of the presence of or concentration of the first allele. Thus, in a preferred embodiment, the signal of the mismatch probe is subtracted from the signal for its corresponding test probe to provide a measure of the signal due to specific binding of the test probe.

The concentration of a particular sequence can then be determined by measuring the signal intensity of each of the probes that bind specifically to that gene and normalizing to the normalization controls. Where the signal from the probes is greater than the mismatch, the mismatch is subtracted. Where the mismatch intensity is equal to or greater than its corresponding test probe, the signal is ignored (i.e., the signal cannot be evaluated).

For each marker analyzed, the genotype can be unambiguously determined by comparing the hybridization patterns obtained for each of the two labels, e.g., color tags employed (FIG. 8). If hybridization is indicated for one color tag to its corresponding allele-specific probe (e.g., “A”) but not for the other color tag (e.g., “G”) (pattern at left in FIG. 8), then the indicated genotype of a diploid organism would be homozygous A/A. If hybridization is indicated only for the other color tag to its corresponding allele-specific probe (e.g., “G”) (pattern at center in FIG. 8), then the indicated genotype of a diploid organism would be homozygous G/G. If hybridization is indicated for both color tags to their corresponding allele-specific probes (pattern at right in FIG. 8), then the indicated genotype of a diploid organism would be heterozygoous (A/G).

Marginal detection of hybridization, indicated by an intermediate positive result (e.g., less than 1%, or from 1-5%, or from 1-10%, or from 2-10%, or from 5-10%, or from 1-20%, or from 2-20%, or from 5-20%, or from 10-20% of the average of all positive hybridization results obtained for the entire array) may indicate either cross-hybridization or cross-amplification, depending on the overall hybridization pattern as indicated in FIG. 8. However, these can be distinguished by the unique pattern observed. Further procedures for data analysis are disclosed in U.S. application Ser. No. 08/772,376, previously incorporated for all purposes.

HuSNP and other marker-specific arrays have been designed and used in genetic studies⁹⁻¹⁰. But the method developed in this study provides several advantages in dealing with many different genetic applications: (1) arrays based on a single generic design can be used to genotype different sets of genetic markers because no specific customized genotyping array is needed; (2) the pre-selected probe sequences synthesized on the tag array help ensure good hybridization results; (3) accurate quantitative measurement of the allele frequency in the tested samples can be achieved. Thus, reliable genotype results can be obtained not only for individual samples, but also for pooled samples. Besides SBE, other assays can be coupled with tag array assay, for example, oligonucleotide ligation assay (OLA)¹⁹⁻²¹, invasive cleavage of oligonucleotide probes assay²², allele specific PCR²³⁻²⁴.

Our current tag chip contains over 32,000 unique tag probes. For most of the genetic application, for example, detecting mutations in one particular gene, it doesn't need such high-density chip. Therefore, smaller chips with fewer tags on the chip are sought after. Alternatively, multiple tags corresponding to one particular marker can be designed as to build the redundancy to the assay to assure accurate genotyping. Or multiple sets of tags for one set of SNPs can be designed, thus multiple samples can be processed and analyzed with one chip. Our current assay uses a two-color labeling scheme. But a four-color labeling/scanning system should warrant the assay can be done in a single tube reaction.

For broader genetic applications, for example, a study needs to genotype 100s to 1000s genetic markers, amplifying the genetic loci with multiplexing PCR is still the best strategy. However, to genotype 1000s to 10,000s markers, pre-amplification of the interested genetic loci will be very labor-intensive and costly. A whole-genome approach should be explored, for example, strategies involved using total human genomic DNA directly, or genomic DNA amplified using some general amplification methods, e.g., primer-extension preamplification, PEP²⁵, or total cDNA. In fact, we have tried to use total human genomic DNA directly as the SBE template in our tag array assay. 24 out of the 38 of the markers that we tested gave good signals (data not shown). Nevertheless, some work is needed to solve both the sensitivity (signal intensity) and specificity (mis-priming) problems before the whole-genome approach becomes really useful.

The invention will be further illustrated by the following non-limiting examples. The content of references cited herein is incorporated herein by reference in its entirety.

### EXEMPLIFICATION

Methods

Collection and Isolation of DNA from Samples

DNA samples were collected by GenNet as part of the ongoing Family Blood Pressure Program. Samples were collected with consent and IRB approval in both Tecumseh, Mich. and Loyola, Ill. FAMILIES. Ascertainment was based on identification of a proband in the top 15^{th} (Tecumseh) or 20^{th} (Loyola) percentile of the community's blood pressure distribution. Full phenotypic information was obtained for each individual. DNA was extracted from 5-10 ml of whole blood taken from each individual using the standard “salting-out” method (Gentra Systems).

Primer Design

For each SNP, primary PCR amplification primers were designed as described previously⁹. The SBE primer was designed in a manner that its 3′ terminates one base before the polymorphic site. Primer 3.0 software package (http://www-genome.wi.mit.edu/cgi-bin/primer/primer3.cgi) was modified and used to pick SBE primers with batch sequences, at a predicted length of 20 (ranging from 18 to 26) nucleotide and melting temperature of 60° C. (ranging from 54° C. to 64° C). The SBE primers were always picked from the forward direction first (i.e. 5′ to the polymorphic site). If the SBE primer can't be picked from the forward direction, reverse direction is tried.

Multiplexing PCR

Specific genomic regions containing the 144 SNPs were amplified with 9 multiplex PCR reactions, each contains 50 ng of human genomic DNA, 0.1 μM of each primer, 1 mM deoxynucleotide triphosphates (dNTPs), 10 mM Tris-HCl (pH 8.3), 50 mM KCl, 5 mM MgCl₂ and 2 units of AmpliTaq Gold (Perkin Elmer) in a total value of 25 μl. PCR was performed on a Thermo Cycler (MJ Research), with initial denaturation of the DNA templates and Taq enzyme activation at 96° C. for 10 minutes; followed by 40 cycles of denaturation at 94° C. for 30 seconds, 57° C. for 40 seconds, and 72° C. for 1 minute and 30 seconds; and the final extension at 72° C. for 10 minutes.

SBE Template Preparation

1 μl of Exonuclease I (Amersham Life Science, 10 U/μl) and 1 μl of Shrimp Alkaline Phosphatase (Amersham Life Science, 1 U/μl) were added to a 25 μl PCR products (see above), and incubated at 37° C. for 1 hour. The enzyme activities were inactivated at 100° C. for 15 minutes. The enzymatically treated samples were applied to a S-300 column (Pharmacia), as to further reduce the residual PCR primers and dNTPs, and replace the buffer with ddH₂O.

Multiplexing SBE Reaction

SBE is carried out in a 33 μl reaction, using 6 μl of the template (see above), 1.5 nM of each SBE primer, 2.5 units of Thermo sequenase (Amersham), 52 mM Tris-HCl (pH 9.5), 6.5 mM MgCl₂, 25 μM of fluorescein-N6-ddNTPs (NEN), 7.5 μM biotin-N6-ddUTP or biotion-N6-dCTP, or 3.75 μM biotin-N6-ddATP, and 10 μM the other cold ddNTPs.

Extension reaction was carried out on a Thermo Cycler (MJ Research), with 1 cycle of 96° C. for 3 minutes, then 45 cycles of 94° C. for 20 seconds and 58° C. for 11 seconds.

After SBE reaction, 9 reactions from each sample were combined and mixed with 30 μl of 100 μg/ml glycogen (Boehringer Mannheim), 18.75 μl of 8 M LiCl (Sigma), and 1125 μl of pre-chilled (−20° C.) ethanol (Abs.), and precipitated by centrifugation at the top speed (Eppendorf centrifuge 5415C) for 15 minutes at room temperature; precipitated samples were dried at 40° C. for 40 minutes and re-suspended in 33 μl ddH₂O.

Tag Array Design and Hybridization

For each tag sequence, two probes were synthesized on the array. One is exactly the designed tag sequence (referred to as a Perfect Match, or PM probe). The other one is identical except for a single base difference in a central position (referred to as a Mismatch, or MM probe). The mismatch probe services as an internal control for hybridization specificity. Over 32,000 20-mer tag probes (and their companions) were chosen¹¹ and fabricated on a 8 mm×8mm size of array. Each probe (feature) occupies a 30 microns×30 microns area. The sets of arrays were synthesized together on a single glass wafer on which 100 arrays were made.

The labeled sample was denatured at 95° C.-100° C. for 10 minutes and snap cooled on ice for 2-5 minutes. The tag array was pre-hybridized with 6×SSPE-T (0.9 M NaCl, 60 mM NaH₂PO₄, 6 mM EDTA (pH 7.4), 0.005% Triton X-100)+0.5 mg/ml of BSA for a few minutes, then hybridized with 120 μl hybridization solution (as shown below) at 42° C. for 2 hours on a rotisserie, at≅40 RPM. Hybridization Solution consists of 3M TMACL (Tetramethylammonium Chloride), 50 mM MES ((2-[N-Morpholino]ethanesulfonic acid) Sodium Salt) (pH 6.7), 0.01% of Triton X-100, 0.1 mg/ml of Herring Sperm DNA, 50 pM of fluorescein-labeled control oligo, 0.5 mg/ml of BSA (Sigma) and 29.4 μl labeled SBE products (see below) in a total of 120 μl reaction.

The chips were rinsed twice with 1×SSPE-T for about 10 seconds at room temperature, then washed with 1×SSPE-T for 15-20 minutes at 40° C. on a rotisserie, at ≅40 RPM. And then wash the chip 10 times with 6×SSPE-T at 22° C. on a fluidic station (FS400, Affymetrix). The chips were stained at room temperature with 120 μl staining solution (2.2 μg/ml streptavidin R-phycoerythrin (Molecular Probes), and 0.5 mg/ml acetylated BSA, in 6×SSPET) on a rotisserie for 15 minutes, at ≅40 RPM. After staining, the probe array was washed 10 times again with 6×SSPET on the FS400 at 22° C. The chips were scanned on a confocal scanner (Affymetrix) with a resolution of 60-70 pixels per feature, and two filters (530-nm and 560-nm, respectively). GeneChip Software (Affymetrix) is used to convert the image files into digitized files for further data analysis.

Clustering Analysis

For a given marker (at a given tag probe position), the intensity of each of the two colors (fluorescein and phycoerythrin) was calculated as the intensity at the perfect match position (PM) minus that at the mis-match position (MM). Negative fluorescein or phycoerythrin intensity values are treated as if they were zero. The Phat values were computed as the ratio of the intensities (fluorescein/fluorescein+phycoerythrin). The Phat values were sorted, and the optimal set of ranges for AA, AB and BB genotypes given the hypothesis of 2 or 3 clusters was considered, subject to the following rules: at most 4 points (outliers) may be excluded from the genotype ranges. For 2 groups, the total range Phat values must be at least 0.3. For 3 groups, the total range Phat values must be at least 0.5. Ranges must be separated by a gap of at least 0.1. The width of a range may be at most 0.4. A score was then computed as: Score=1−(sum of range widths/total range)−(outliers*0.1).

The set of ranges with the best score was found and used to call genotypes. This score increases with narrow ranges, while decreases with the number of points that are left out of any range. Therefore, it tends to be optimal when all the phat values are contained within relatively small ranges.

ABI Sequencing to Determine Genotypes

To independently confirm the genotypes called from the tag array assay, three samples (904957000000, 904896000000, and 904889000000) were sequenced using gel-electrophoresis based method. Samples were amplified for all sites with T7 and T3 tagged primers, using standard PCR cycling conditions (2.5 μl of 20 ng/μl DNA, 0.375 μl of 20 μM primer (X2), 1.5 μl of 10×PCR buffer, 0.9 μl 25 mM Mg²⁺, 0.15 μl 10 mM dNTPs, 0.25 μl 10 U/μl Taq DNA Polymerase (Sigma), brought up to 15 μl with ddH₂O per tube). Some products were sequenced directly, while a M13 nesting strategy was used due to the close proximity of the polymorphic base to the primer end. Samples from the initial amplification were diluted 1:50 with ddH₂O, and amplified with M13F-T7 (TGTAAAACGACGGCCAGTTAATACGACTCACTATAGGGAGA; SEQ ID NO: 9) and M13R-T3 (AACAGCTATGACCATGAATTAACCCTCACTAAAGGGAGA; SEQ ID NO: 10) primers using standard PCR conditions. All PCR products were cleaned with Exonuclease I (Amersham 0.15 μl of 10 U/μl per well) and Shrimp Alkaline Phosphatase (Amersham, 0.30 μl of 1 U/μl per well) in a volume of 10 μl. Dye terminator sequencing using a M13R primer (AACAGCTATGACCATG; SEQ ID NO: 11) or T7 primer (TAATACGACTCACTATAGGGAGA; SEQ ID NO: 12) on an ABI377 (Perkin Elmer) using Big Dyes (Perkin Elmer) was performed to determine the genotype status for each SNP in all three individuals. Trace files were read with Edit View 1.0 (Perkin Elmer) software.

### EXAMPLE 1

DNA from a individual is isolated, and amplified with primers from 15 previously-characterized (i.e., known) SNPs. Amplification is allowed to proceed as described in Hudson, T. J. et al. (Science 270:1945-1954 (1995)) and Dietrich et al. (Dietrich, W. F. et al., Nature 380:149-152 (1996); Dietrich, W. F. et al., Nature Genetics 7:220-245; Dietrich, W. et al., Genetics 131:423-447 (1992)). For example, in a 50 μl reaction volume, 0.5 ng of template nucleic acid/target polynucleotide is added to 1 μM forward amplification primer, 1 μM reverse amplification primer, 200 μM dGTP, 200 μM dTTP, 200 μM dATP, 3.5 mM MgCl₂, 1.0 mM Tris-HCl (pH 8.3), 50 mM KCl, 0.02 μM molecular probe, and 0.25 units of polymerase enzyme. The reaction mixture can then be subjected to a two-step amplification process, performed on a Tetrad (MJ Research, Watertown, Mass.), with the conditions: denaturation at 94° C. for 60 seconds, followed by an annealing/extension step at 53°-56° C. for one minute. The denaturation and annealing/extension steps are repeated for 40 cycles. Alternatively, a three-step thermocycling reaction can be used, such as 94° C. for 60 seconds, followed by annealing at 53°-56° C. for 30 seconds, followed by extension at 72° C. for one minute the three steps being repeated for 40 cycles. This may be followed by an optional extension step at 72° C. for five minutes.

After amplification is complete, locus-specific tagged oligonucleotides specific for the 10 SNPs are added, and are allowed to hybridize to the amplification products.

Reagents for a single base extension reaction are then added, where each of the four ddNTPs is labeled with a different fluorophore. Single base extension is then performed as described by Kobayashi et al. (Mol. Cell. Probes 9:175-182 (1995)).

After the reaction is complete, the reaction products are placed in contact with the universal array, and the reaction products allowed to hybridize, each product to its appropriate oligonucleotide tag on the array. The chip is then assayed in a fluorometer, and the wavelength emitted at each address in the array is recorded. From this data, the genotype at each individual SNP is determined.

### EXAMPLE 2

Two alleles of template were mixed at ratios of 1:30, 1:10, 1:3, 1:1, 3:1, 10:1, and 30:1. These were labeled with different color labels by single-base extension reaction and hybridized to a tag array. A correlation was observed between the signal intensity ratio and the template concentration ratio over a 900-fold dynamic range. See FIG. 2.

### EXAMPLE 3

A set of tag sequences is selected such that the tags are likely to have similar hybridization characteristics and minimal cross-hybridization to other tag sequences. An oligonucleotide array of all of the tags is fabricated. The design and use of such a 4,000-20mer-tag array for the functional analysis of the yeast genome has been described (1). More recently, Affymetrix designed and fabricated an array with a set of more than 16,000 such tags. The tag sequence synthesized on the chip can be 20-mer, 25-mer, or other lengths.

### EXAMPLE 4

Marker specific primers are used to amplify each genetic marker (e.g. SNP). A multiplex PCR strategy is used to amplify these markers from genomic DNAs of tested individuals (2). After PCR amplification, excess primers and dNTPs are removed enzymatically. These enzymatically treated PCR products then serve as templates in the next SBE reaction. Please note that these templates (PCR products) are double stranded, which are different from the templates used in other protocols (3, 4). For example, in Minisequencing (3) and Genetic Bit Analysis (GBA, 4), a double stranded template has to be converted to a single stranded template prior to the base extension reaction. The methods used for this conversion are costly, laborious, and hard to automate.

### EXAMPLE 5

In the protocol described below, an SBE primer is designed for each genetic marker which terminates 1 base before the polymorphic site. However, other primer design schemes can be used. The primer for each marker is tailed with an unique tag which is complementary to a specific probe sequence synthesized on the tag chip. The extension reaction is multiplex, in which SBE primers corresponding to multiple markers were added in a single reaction tube, and extended in the presence of pairs of ddNTPs labeled with different fluorophores, e.g. for an A/C variant, there might be a ddATP-red and DDCTP-green.

### EXAMPLE 6

The resulting mixture is hybridized to the tag array. Each tag corresponds to a single marker. The ratio of the intensities of the colors indicates the genotype (or the allele frequency, ranging from 0% to 100%) of the samples tested.

### EXAMPLE 7

SBE template preparation: Marker specific primers are used to amplify each single nucleotide polymorphism (SNP). A multiplex PCR strategy is used to amplify these SNPs (Science 280:1077-1082, 1998).

Multiplex PCR:

Multiplex PCR reaction is carried out with AmpliTaq Gold and 25 primer pairs in a 25 μl reaction volume. SNPs with same base composition at the polymorphic site (i.e. A/G, T/C, etc) are pooled together.

PCR reagents:

| | |
|---|---|
| 10XPCR Multiplex Buffer (II): | 100 mM |
| | Tris/HCl (pH 8.3) |
| | 500 mM KCl |
| | 25 mM dNTPs |
| F & R Primers (for each primer, the conc. is 1 μM) | 20 ng/μl |
| | Genomic DNA |

Multiplex PCR reaction (25 ul)

| | |
|---|---|
| Primer Mix (1 μM each) | 2.5 μl |
| Genomic DNA (20 ng/μl) | 2.5 μl |
| 10XPCR Buffer II | 2.5 μl |
| 25 mM MgCl₂ | 5 μl |
| 25 mM dNTPs | 1 μl |
| AmpliTaq Gold (5 U/μl) | 0.4 μl |
| ddH₂O | up to 25 μl |

PCR conditions

| | | |
|---|---|---|
| 40 cycles: | 96° C. | 10 min |
| | 94° C. | 30 sec |
| | 57° C. | 40 sec |
| | 72° C. | 1 min 30 sec |
| | 72° C. | 10 min |
| | 4° C. | O/N |

Enzymatic treatment of PCR products to degrade and de-phosphorylate the unused primers and dNTPs, respectively:

To a 25 μl PCR products, add 1 μl of Exonuclease I (Amersham Life Science, 10 U/μl) and 1 μI of Shrimp Alkaline Phosphatase (Amersham Life Science, 1 U/μl), and incubate at 37° C. for 1 hour. Inactivate the enzyme activities at 100° C. for 15 minutes. Apply the sample to a S-300 column (Pharmacia), to further reduce the residual PCR primers and dNTPs, and replace the buffer with ddH₂O. The sample is ready for next SBE reaction.

Single Base Extension (SBE):

An SBE primer is designed for each SNP which terminates 1 base before the polymorphic site. The primer for each SNP is tailed with a unique tag which is complementary to a specific probe sequence on the tag chip. The SBE reaction is also multiplexed at 25-plex.

Reaction Mixture (33 μl):

| | | |
|---|---|---|
| Template (see above) | 6 | μl |
| SBE Primer mix (20 nM for each primer) | 2.5 | μl |
| 5X Thermo Sequenase buffer | 6.6 | μl |
| Bio-(d)dNTP(X nmol/μl*, NEN) | 0.5 | μl |
| Flu-ddNTP(1 nmol/μl, NEN) | 0.8 | μl |
| Other two cold - ddNTPs(1 nmol/μl, Biopharmacia) | 0.3 | μl each |
| Thermo Sequenase(6.4 U/μl) | 0.4 | μl |
| (Amersham) | | |
| ddH₂O | up to 33 | μl |

*X = 0.5 when it is Bio-ddUTP or bio-dCTP(0.5 mM), or X = 0.25 when it is bio-ddATP (0.25 mM)

PCR program:

| | | |
|---|---|---|
| 96° C. | 3′ | 1 cycle |
| 94° C. | 25″ | |
| 58° C. | 11″ | 45 cycles |
| 4° C. | forever | |

Precipitation:

After SBE reaction, we combined 9 tubes for each sample, mix with 30 μl of 100 μg/ml glycogen (Boehringer Mannheim), then precipitated with 18.75 μl of 8 M LiCl, and 1125 μl of pre-chilled (−20° C.) ethanol (Abs.). Mix well; then centrifuge at the top speed (Eppendorf centrifuge 5415C) for 15 min at room temperature; Decant the supernatant, and dry the samples at 40C for 40 min, re-suspend the samples in 33 μl ddH2O, now it is ready for hybridization.

Hybridization:

The prepared sample is denatured at 100° C. for 10 minutes and snap cooled on ice for 2-5 minutes. The universal tag chip is pre-hybridized with 6×SSPE-T (0.9 M NaCl, 60 mM NaH₂PO₄, 6 mM EDTA (pH 7.4), 0.005% Triton X-100)+0.5 mg/ml of BSA, then hybridized with 120 μl hybridization solution (as shown below) at 42° C. 2 hours on a rotisserie, at ≅40 RPM.

The hybridization solution contains:

| | |
|---|---|
| 5M TMACL | 72 μl |
| 0.5M MES (pH 6.7) | 12 μl |
| 1% Triton X-100 | 1.2 μl |
| HS DNA (10 mg/ml) | 1.2 μl |
| Flu-c213 (5 nM) | 1.2 μl |
| BSA (20 mg/ml) | 3.0 μl |

Plus 29.4 μl prepared sample (see above).

Post-Hybridization Wash:

Rinse the chip with 1×SSPE-T 10″ twice first, then wash with 1×SSPE-T for 15-20 min at 40° C. on a rotisserie, at 40 RPM. And then wash on a fluidic station (FS400, Affymetrix) 10 times with 6×SSPET at 22° C.

Staining:

Stain the chip at room temperature with 120 μl staining solution (2.2 μg/ml streptavidin R-phycoerythrin (Molecular Probes), and 0.5 mg/ml acetylated BSA, in 6×SSPET) on a rotisserie for 15 minutes, at ≅40 RPM. After staining, the probe array was washed 10 times again with 6×SSPE-T on the FS400 at 22° C.

Scanning:

The chips were scanned on a confocal scanner (Affymetrix) with a resolution of 60-70 pixels per feature, and two filters (530-nm and 560-nm, respectively). GeneChip Software (Affymetrix) is used to convert the image files into digitized files for further data analysis.

### EXAMPLE 7

Genotyping with High-Density Oligonucleotide “Tag” Arrays

A genotyping method based on the use of a high-density “tag” array that contains over 32,000 pre-selected 20-mer oligonucleotide probes, combined with marker-specific PCR amplifications and single base extension (SBE)¹⁻² reactions has been developed. We have used this method to genotype a collection of 144 single-nucleotide polymorphism (SNPs) identified from 49 hypertension candidate genes³. First, marker-specific primers were used in multiplex PCR reactions to amplify specific genomic regions containing the SNPs. The PCR amplified DNA products were then used as templates in SBE reactions. Each SBE primer comprises a 3′ portion and a 5′ portion. The 3′ portion is complementary to the specific SNP locus and terminates one base before the polymorphic site. The 5′ portion comprises a unique sequence, which is complementary to a specific oligonucleotide probe synthesized on the “tag” array. The extension reaction is multiplex, with SBE primers corresponding to multiple SNPs in a single reaction tube. The primers are extended in the presence of two-color labeled ddNTPs, and the resulting mixture is hybridized to the tag array. The intensity ratio of the two colors was used to deduce the genotypes of the samples tested.

The tag array strategy begins with an array of tag sequences selected in a manner that all tag probes are in the same length, e.g. 20-nucleotide long, with similar melting temperature and G-C content, and the lowest sequence homologous among each other¹¹. Therefore, these tags are likely to have similar hybridization characteristics and minimal cross-hybridization to other tag sequences.

The design and use of a 4,000-tag array for the functional analysis of yeast Saccharomyces cerevisiae genes¹¹ and drug sensitivity studies¹² have been described. More recently, we have designed and fabricated an array that contains more than 32,000 such tags, and developed it as a genotyping tool, in combination with marker-specific PCR amplifications and SBE reactions.

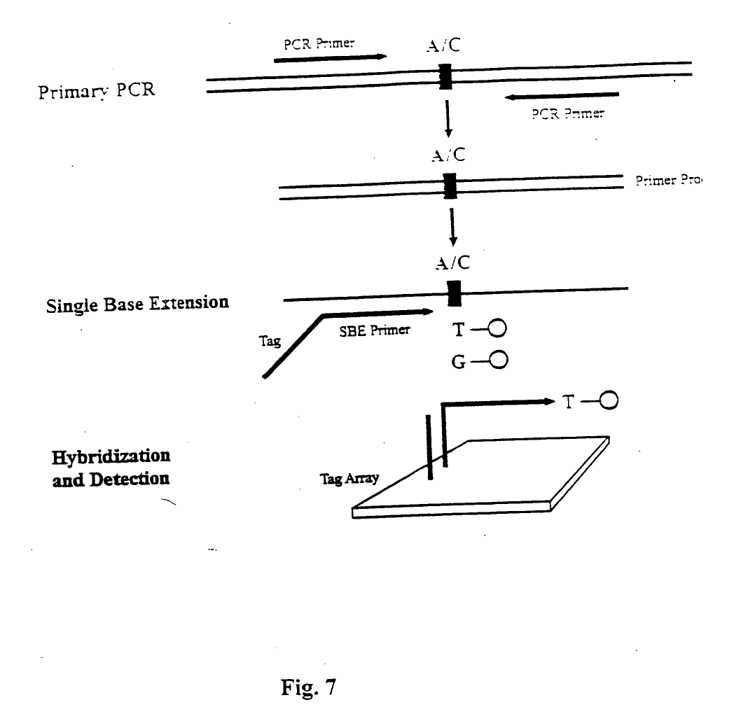

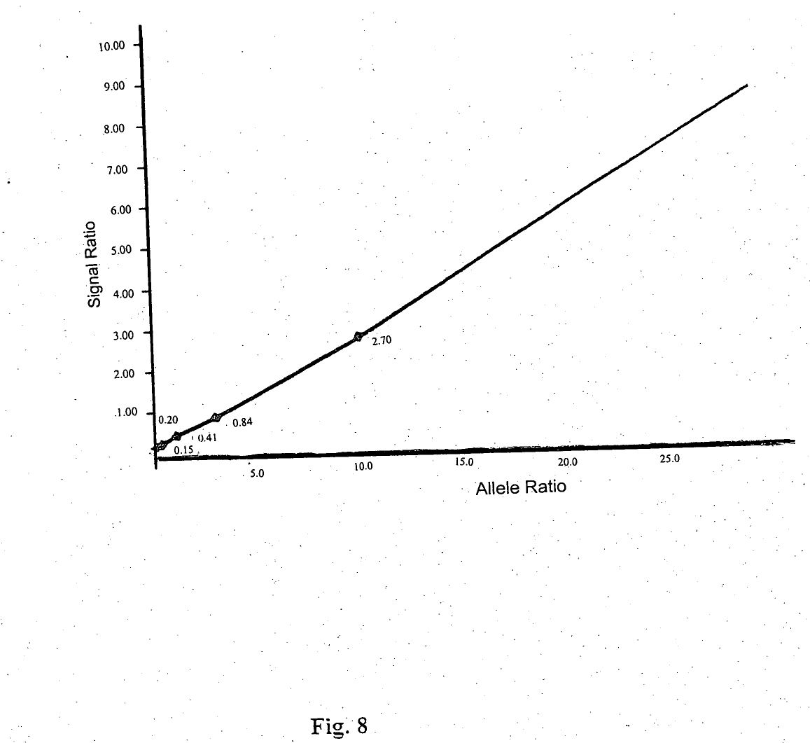

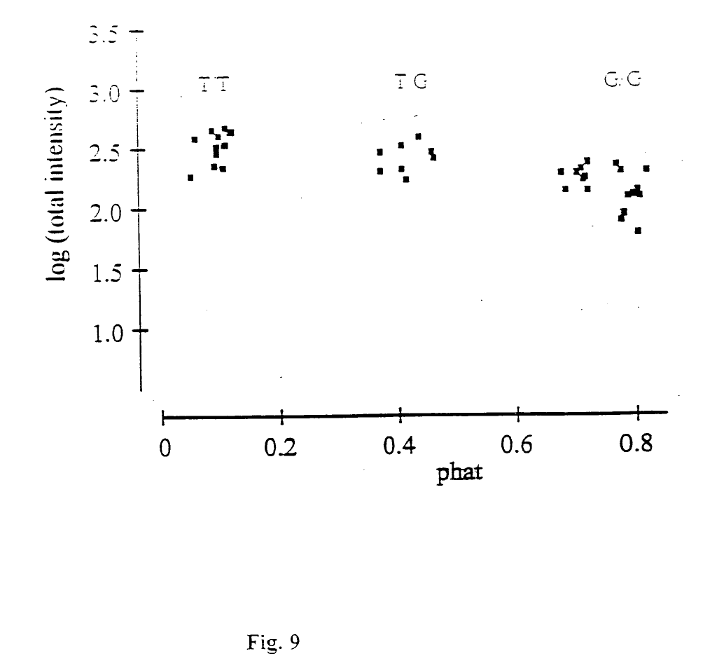

As shown in FIG. 7, marker specific primers are designed and used to amplify each single nucleotide polymorphism (SNP). A multiplex PCR strategy is used to amplify these SNPs from genomic DNAs⁹. In general, SNPs with same base composition at the polymorphic site (e.g. all the A/G polymorphisms) are grouped together. After PCR amplification, excess primers and dNTPs are degraded and de-phosphorylated using Exonuclease I and Shrimp Alkaline Phosphatase, respectively. These enzymatically treated PCR products (double-stranded) are then served as templates in the SBE reaction. A SBE primer is designed for each genetic marker, which terminates one base before the polymorphic site. Each primer is tailed with a unique tag that is complementary to a specific probe sequence synthesized on the tag array. The extension reaction is multiplex, in which SBE primers corresponding to multiple markers (up to 56 markers that we have tested so far) were added in a single reaction tube, and extended in the presence of pairs of ddNTPs labeled with different fluorophores, e.g. for an A/G variant, biotin-labeled ddATP and fluorescein-labeled ddGTP are used. The resulting mixture of SBE reactions is hybridized to the tag array. Each tag hybridizes to a specific probe position on the chip. The ratio of the intensities of the colors indicates the genotype (homozygous wild type, or homozygous mutant, or heterozygous) or the allele frequency (ranging from 0% to 100%) in the samples tested.

In a comparison of the results of using single-stranded and double-stranded PCR products as the templates in the current SBE/tag array assay, no significant difference was found (data not shown). However, in previously published protocols of minisequencing¹³⁻¹⁵ and genetic bit analysis₁₆₋₁₈, a double-stranded template has to be converted to a single-stranded template prior to the base extension reaction. The methods used for this conversion were costly, laborious, and hard to automate.

The tag array assay provides a fairly accurate quantitative measurement of the allele frequency in samples tested. As shown in FIG. 2, we have synthesized two artificial SBE templates. They are identical, except the 21^{st} position: T in template-T, and G in template-G. We then mixed the two templates at ratios of 1:10, 1:3, 1:1, 3:1, 10:1, and 30:1, which is a 300-fold dynamic range. Six SBE primers, which have the same 3′ portion (the portion complementing to the template sequence) but different 5′ portion (the portion complementing to the tag probes on the tag arrays) were designed (FIG. 2), and extended in the presence of the SBE templates mixed at different ratios, and biotin-labeled ddATP and fluorescein-labeled ddCTP. As shown in FIG. 8, the intensity ratio of the two colors and the template concentration ratio (i.e. the allele frequency) appears to form a fairly good linear correlation in the 300-fold dynamic range that we tested.

To further test the robustness and the efficiency of the tag array/SBE assay method for genotyping application, we set out to type a portion of the SNPs that we had identified from a large-scale polymorphism screening study with the hypertension candidate genes³. Initially, we selected 173 SNPs from 56 hypertension candidate genes. These SNPs were chosen for their being occurred in promoter regions, or splicing junctions, or coding regions in which the nucleotide changes caused amino acid changes. We reason that these SNPs can be the good candidates for being the functional mutations predisposed to the disease. Therefore, the assay developed in this study could then be used in large-scale association studies in hypertension. PCR primers were designed and tested individually for these 173 SNPs. 8 of them (4.6%) failed to amplify. SBE primers were then designed for the remaining 165 SNPs. A multiplexing PCR and multiplexing SBE assay was developed with a complexity of 9 to 28 markers in each reaction and a total of 9 reactions for the 165 markers. 21 of them (12.7%) failed in the multiplexing PCR and multiplexing SBE assay. Therefore, 144 markers from 49 genes passed the assay development. The gene location, polymorphic sites, and the designed primers for these 144 markers were summarized in Table 1.

We then genotyped 44 individuals using 44 tag arrays. Good hybridization signals were obtained in 96.5% (6116/6336 (144×44)) of the cases. The signal intensity values from the hybridization results were used in clustering analysis for each of the 144 markers. Genotypes for each individual at the 144 loci were assigned automatically based on the clustering results, with some manual editing. Data Desk 6.0 (Data description, Inc.) was used to manually display the clustering analysis results (of the intensity ratios of the two colors). Overall, 80-85% of the markers form good cluster(s).

We have performed the gel-based DNA sequencing to determine the genotypes at 115 loci in 3 of the 44 individuals (see Methods). Comparison of the ABI sequencing results and the chip results resulted in 14 discrepancies (4%), out of 115×3=345 genotype calls. Most of the discrepancies occurred in cases where one method called homozygous, while the other method called heterozygous. In one case (marker ICAM1ex6.254), where the ABI sequencing method called G/G, but the tag array/SBE assay method called A/A in all the three individuals, we believe the discrepancies are due to mis-priming of the SBE primer to adjacent sequences.

We also tested the reproducibility of the tag array/SBE assay genotyping method. We repeated the multiplexing PCR, SBE and the chip hybridization experiments in 4 individuals. The ratios of the two colors (for each of the 144 markers) in the replicated experiments are not all exactly the same, but they all fall into the same cluster (i.e. giving the same genotype call). Therefore, we didn't find any discrepancy in the genotyping call of duplicated samples.

TABLE 1

| Gene/Exon/ | | | | |
|---|---|---|---|---|
| Position | SNP Flanking Sequence | Forward Primer | Reverse Primer | SBE Primer |
| AADDEX10.246 | TTCCGAGGAA(G/T)GGCAGAATGG | GACGAAGCTTCCGAGGA | GGGACTGCTTCCATTCTGC | AGAGTCTATAAGCATCGTCGGGCGACGAAGCTTCCGAGGAA |
| AADDEX13.173 | CAGAAGGGCT(C/G)TGAAGGTGAG | GAGAGGAAGCAGAAGGGC | GACCACAAGCACTCACCTTC | TCAGACAATTCTATACGCGGTGGAGAGGAAGCAGAAGGGCT |
| ACEEX13.138 | TGCTGGTCCC(C/T)AGCCAGGAGG | GCACCCTCTGCTGGTCC | TGACTGTCACCTGTTGGGA | TCGTGAGTTGTCCTGCTGCAGCACCCTCTGCTGGTCCC |
| ACEEX13.151 | CCAGGAGGCA(T/C)CCCAACAGGT | GCACCCTCTGCTGGTCC | TGACTGTCACCTGTTGGGA | GCCTGTAATGGTGGATCTCAGTCCCCAGCCAGGAGGCA |
| ACEEX13.202 | AACAACCAGC(A/G)GCCAGACAAC | AGCCAGGCAACAACCAG | GTGGGTGGTTGTCTGGC | GATCTGTCTGACGCTGTATGGCAGCCAGGCAACAACCAGC |
| ACEEX15.144 | AACGGGCAGC(G/A)CTGCCTGCCC | AGGACCTAGAACGGGCAG | TCCTGGGCAGGCAGC | CGTGATAATGCGTCTCGTAGCAGGACCTAGAACGGGCAGC |
| ACEEX17.19 | AGCCATTCAA(C/A)CCCCTACCAG | TGGAGCTCAAGCCATTCA | CGTCAGATCTGGTAGGGGG | CATTATCGGACATGCTCACTTGGAGCTCAAGCCATTCAA |
| ACEEX17.52 | TGATGGCCAC(A/G)TCCCGGAAAT | GACGAATGTGATGGCCA | GGTCTTCATATTTCCGGGAT | ATGATGAGCCGTGATGACCCCTGACGAATGTGATGGCCAC |
| ACEEX18.130 | CACTCTACCT(C/G)AACCTGCATG | GAGCTGCAGCCACTCTACC | CGTAGGCATGCAGGTTG | TACATCGCTTGCATGAGTGTGAGCTGCAGCCACTCTACCT |
| ACEEX21.150 | CATGAGGCCA(T/C)TGGGGACGTG | CGGCTTCCATGAGGCC | GGCTAGCACGTCCCCAA | GATCTGGCTTCAACTGTATGCCGGCTTCCATGAGGCCA |
| ACEEX22.19 | TGACATCAAC(T/G)TTCTGATGAA | TTGCAGAGCATGACATCAA | AAGGGCCATCTTCATCAGA | TGCCTAGCTTTCCATATCGGCCTTGCAGAGCATGACATCAAC |
| ACEEX24.118 | CCAAGGAGGC(C/T)GGGCAGCGCC | CATCTACCAGTCCAAGGAGG | TCACCCAGGCGCTGC | TATCTCGCTTGCTATCAACGATCTACCAGTCCAAGGAGGC |
| ACEEX24.16 | CCAGGTACTT(T/C)GTCAGCTTCA | TCGCTCGCTCCAGGTACT | GGAACTGGATGATGAAGCTGA | GCCTAAGCTCTGTCGTGATTCGCTCTGCTCCAGGTACTT |
| ACEEX26.154 | CTCAGCCAGC(G/A)GCTCTTCAGC | CTGGGCCTCAGCCAG | GCGGATGCTGAAGAGCC | TCTATTGCTGTTCGGCGGCAACCCTGGGCCTCAGCCAGC |
| ACEEX26.174 | CATCCGCCAC(C/A)GCAGCCTCCA | TCTTCAGCATCCGCCA | GCCGGTGGAGGCTGC | AGCAGAGATGGACAGACCTCCTCTTCAGCATCCGCCAC |
| ACEEX26.205 | CACGGGCCCC(A/C)GTTCGGCTCC | CACTCCCACGGGCCC | CACCTCGGAGCCGAACT | GCTGGCGGTTCATGCAATCTTCCACCTCGGAGCCGAAC |
| ACEEX26.224 | CCGAGGTGGA(G/A)CTGAGACACT | TCGGCTCCGAGGTGG | CACCTCAGGAGTGTCTCAGC | TATCTGCGTTGCTGACGTGCCAGTTCGGCTCCGAGGTGGA |
| ACEEX8.106 | AGGATCTGCC(C/T)GTCTCCCTGC | CCTGCAGTACAAGGATCTGC | CCCGACGCAGGGAGA | GATCCGTATGTCGAATGGCTCTGCAGTACAAGGATCTGCC |
| ACEP.-3892 | TAAGGGGGGG(T/C)TGCTGTACAT | CCACTGAGGATAAGGGGG | GAAGATATTTGCAAAGTATGTACAGC | CCAGAGGTGCGGTCACATATCACTGAGGATAAGGGGGGGG |
| ACEP.-5466 | TATAGTATAT(A/C)TATGCCCAGC | GTCATGCCATGTCACATATATTATAGT | GACCATGGCTGGGCAT | GCATCTTCGCCAGCTATATTGGTTGACCATGGCTGGGCATA |
| ACEP.-93 | GCTGGGGACT(T/C)TGGAGCGGAG | AGGAACCTCGGCCCG | GCTTCCTCCTCCGCTCC | CACTTACGGCCATGCTGAATCCCGCGCCGCTGGGGACT |
| ADDBEX15.85 | AGTTCTTCAG(C/T)GTTGCCCTCC | CCGTGTGCGAGTTCTTCA | CCAGATGTGGAGGGCAA | CACTGTACGCACTGGAGCTACGTGTGCGAGTTCTTCAG |
| ADDBEX3.138 | CTCAGAGGAC(G/A)ACCCCGAGTA | TGACCGCTTCTCAGAGGA | GCGCATGTACTCGGGG | GTGTGCATTGAGTCTATGACTTTGACCGCTTCTCAGAGGAC |
| ADRBJEX1.416 | GGCCATCGCC(T/C)GGACTCCGAG | CATCGTGGCCATCGC | CTGGAGTCTCGGAGTCCA | CGTCTCATGCCTGCGTATAGTGGTCATCGTGGCCATCGCC |
| ADROMEX3.81 | GGATGTCCAG(C/G)AGCTACCCCA | GGAACTGCGGATGTCCA | GCCCGGTGGGGTAGC | TACATCATTGCGAGTCATGGAAGAGGGAACTGCGGATGTCCAG |
| AEIEX14.159 | CTTCTTTGCC(A/T)TGATGCTGCG | CCGGTACCTTCTTCTTTGC | TGAACTTGCGCAGCATC | ATACGCTCTGCCATACGTGAGCCGGTACCTTCTTCTTTGCC |
| AEIEX4.36 | TCAGCTCACG(A/C)CACCGAGGCA | CCGACCTCTGGTTTTCAGC | TGGCTGTTGCCTCGGT | TTGCGCCATTTGGACATGCTACCTCTGGTTTTCAGCTCACG |
| AEIEX4.89 | GGGTACCCAC(A/G)AGGTGAGGAC | CACACCCGGGTACCCA | GGCTGGGGTCCTCACC | GCCTGATATTCATTCACAGCACATCACACCCGGGTACCCAC |
| AGT.385 | CCGTTTCTCC(T/C)TGGTCTAAGT | GACTTTGAGCTGGAAAGCAG | CATGCAGCACACTTAGACCA | TTTCGTGCTTTGGAGACAGCAATGGTCGGGATGCTGGC |
| AGTEX2.354 | GGATGCTGGC(C/T)AACTTCTTGG | TGGTCGGGATGCTGG | CGGAAGCCCAAGAAGTTG | TTTCGTGCTTTGGAGACAGCAATGGTCGGGATGCTGGC |
| AGTEX2.755 | TTCACAGAAC(T/G)GGATGTTGCT | CGTCTCTGGACTTCACAGA | TCTCAGCAGCAACATCCA | TGCCGTGTTGGTGCTTCACACTCTCTGGACTTCACAGAAC |
| AGTEX2.827 | TGCTCCCTGA(T/C)GGGAGCCAGT | AGACTGGCTGCTCCCTG | TCCACATGGCTCCA | TCGTCCACTTTAGCATGATGAAGACTGGCTGCTCCCTGA |
| AGTEX5.376 | GGAAAGCAGC(C/G)GTTTCTCCTT | GACTTTGAGCTGGAAAGCAG | CATGCAGCACACTTAGACCA | TACATACTTGCAGTGCGTTCACTTTGAGCTGGAAAGCAGC |
| AGTEX5.385 | CCGTTTCTCC(T/C)TGGTCTAAGT | GACTTTGAGCTGGAAAGCAG | CATGCAGCACACTTAGACCA | CGTCGTGCTGCGTGACTATAGGAAAGCAGCCGTTTCTCC |
| AGTEX5.641 | TCGGTTTGTA(T/G)TTAGTGTCTT | GCATTGCCTTCGGTTTGT | TCATGTTCTTACATTCAAGACACTAAA | TGAGAGTCTGTTCTTAGGCCCATTTTGCATTGCCTTCGGTTTGTA |
| AGTEXP1.101 | CTGTGCTATT(G/C)TTGGTGTTTA | CTTTCAATCTGGCTGTGCTAT | GGGGAGACTGTTAAACACCAA | TACATAATTGCCATGACGGGTTCAATCTGGCTGTGCTATT |
| AGTEXP2.160 | CCTTGGCCCC(G/A)ACTCCTGCAA | TGGGAACCTTGGCCC | ACCGAAGTTTGCAGGAGTC | GAGAATGCTGTATAGTGTCCTTTCTGGGAACCTTGGCCCC |
| AGTEXP2.203 | ACCCTGCACC(G/A)GCTCACTCTG | TGTGTAACTCGACCCTGCAC | CTGCTGAACAGAGTGAGCC | CGTCTCGCTGGTCACTAATGGTGTAACTCGACCCTGCACC |
| AGTEXP2.35 | CTGCACCTCC(G/A)GCCTGCATGT | TCTGCCCTCTGCACCTC | CAGGGACATGCAGGCC | GATCTCTGTGAAGTTAGTGCCCTCTGCCCTCTGCACCTCC |
| AGTEXP3.144 | TAAATAGGGC(C/A)TCGTGACCCG | CACCCCTCAGCTATAAATAGGG | CGGCAGCTTCTTCCCC | TATAAAGATTGCGGTCAGGCCCCTCAGCTATAAATAGGGC |
| AGTEXP3.158 | TGACCCGGCC(A/G)GGGGAAGAAG | CACCCCTCAGCTATAAATAGGG | CGGCAGCTTCTTCCCC | CCAGTCGGTGTAGCAGCAATTAGGGCCTCGTGACCCGGCC |
| AGTEXP3.173 | AAGAAGCTGC(C/T)GTTGTTCTGG | GCCAGGGGAAGAAGCTG | GCTGTAGTACCCAGAACAACG | GTGTGCTCTTCTCGCTGCAAGCCAGGGGAAGAAGCTGC |
| ALDREDEX1.162 | CAAGATGCCC(A/T)TCCTGGGGTT | ACGGCGCCAAGATGC | AGGTACCCAACCCCAGG | ATACCGGCTGCTACACAGTGAACGGCGCCAAGATGCCC |
| ALDREDEX1.71 | GTACGCGCCG(C/G)GGCCAAGGCC | GCTATTTAAAGGTACGCGCC | TACGGTGCGGCCTTG | CAAATAGTGTGCGAGGATCTGCTATTTAAAGGTACGCGCCG |
| ALDREDEX13.28 | TCGCTGGCTT(A/T)GCTGTGGTGC | GCCCTCTCGCTGGCT | CATGGTACGTGCACCACAG | TGAGACATTGTGCAAATCGGACATGTGCCCTCTCGCTGGCTT |
| ANPEX3.33 | TTTGCAGTAC(T/C)GAAGATAACA | ATATGTCTGTGTTCTCTTTGCAGT | CTCCCTGGCTGTTATCTTCA | GATAGCAGTTCACTACCTGGGTCTGTGTTCTCTTTGCAGTAC |
| APOA2.249 | TCCTGTTGCA(T/C)TCAAGTCCAA | TTGGAATCCTGCTTCCTGT | GATCTGAGGTCCTTGGACTTG | GGCATCACTGGTTACGTCTGATCTGAGGTCCTTGGACTTGA |
| APOA4.3058 | AGGAACAGCA(T/G)CAGGAGCAGC | AGCAACAGCAGGAACAGC | CACCTGCTCCTGCTGC | GTCTGACTTGAGTTACATGGGAGCAACAGCAGGAACAGCA |
| APOCIEX1.462 | TTCTGTCGAT(C/G)GTCTTGGAAG | TGGTGGTGGTTCTGTCGA | TCCCACTTTTACCTTCCAAGA | GGTCTTCCTATATGTGCGCGTCCTGGTGGTGGTTCTGTCGAT |
| APOC2.804 | CTTTCTCCCC(A/T)GGGACTTGTA | ACCATCTGTGCTTTCTCCC | TCATGGCTGCTGTGCTT | TGAGAAGTTGTGAAGATCCCTAACCATCTGTGCTTTCTCCCC |
| APOC2.819 | CTTGTACAGC(A/C)AAAGCACAGC | ACCATCTGTGCTTTCTCCC | TCATGGCTGCTGTGCTT | GCCAGGCGTTCAGATGCAATCCCAGGGACTTGTACAGC |
| APOC4.3162 | CTGGGTCCGC(T/G)CACCAAGGCC | AGGGACCTGGGTCCG | AGGAACCAGGCCTTGGT | GCTGGTCGTGGTCCAATCATTGAGGGACCTGGGTCCGC |
| APOER2EX12.68 | ACTGTCCAGC(A/C)TTGACTTCAG | CAAGCTACACCAACTGTCCAG | TCTGTTGCCTCCACTGAAG | GACCATGCTGGCTTACCTGTAAGCTACACCAACTGTCCAGC |
| AT2EX3.807 | GGGAAGAACA(G/A)GATAACCCGT | GACGAATAGCTATGGGAAGAAC | ACTTGGTCACGGGTTATCC | TGGCATCGTTTCACCTGCTGGACGAATAGCTATGGGAAGAACA |
| APEX2.154 | AACTACCTGC(C/T)GTCGCCCTGC | TGCCAGGAGGAGAACTACCT | GGACTGGCAGGGCGA | TATCATTCTGTGGTCGGCGCCCAGGAGGAGAACTACCTGC |
| AVPR2EX2.129 | GCGGAGCTGG(C/T)GCTGCTCTCC | CTAGCCCGGGCGGAG | CCACAAAGACTATGGAGAGCAG | GTGGATCTTGATGTAATGCCTAGCCCGGGCGGAGCTGG |
| AVPR2EX2.444 | CCCATGCTGG(C/T)GTACCGCCAT | TGCCGTCCCATGCTG | CCACTTCCATGGCGGTA | GCCGTCAATGGGTGCTCAATATCTGCCGTCCCATGCTGG |
| BIR.1521 | GGCACTTTGA(C/T)GGTGTTGCCA | AGTGGTGTGGGCACTTTG | CTACTCCAAGTTTGGCAACAC | GCCAGTCATTCCACGTATATAGAGTGGTGTGGGCACTTTGA |
| BIR.2463 | TACCTGGGCT(T/C)GGCAGGGTCC | GGCACGGTACCTGGGC | CGCCTGGCAGAGGACC | GCCAGCCATGTGTCGAATGAGGGCACGGTACCTGGGCT |
| BRS3EX1.730 | CATCTATATT(A/C)CTTATGCTGT | GAAGCATTGTGTGCCATCTA | CCACTGAAATGATCACAGCA | ATCTCAGAGTGGCATCGGATAGAAGCATTGTGGCCATCTATATT |
| CAL/CGRPEX4.30 | TTCCCTGCAG(C/A)CTGGACAGCC | CTGGTATGTGTTTTCCCTGC | CTTAGATCTGGGGCTGTCC | GTCTGCAATTATCGGCTGTGTCTGGTATGTGTTTTCCCTGCAG |
| CaR AA1011 | GACCCGACAC(C/G)AGCCATTACT | CGATACGCTGACCCGACA | GCAGCGGGAGTAATGGC | GGTCTGCATTCGCTGATATGAGCGATACGCTGACCCGACAC |
| CaR AA990 | CATGGCCCAC(G/A)GGAATTCTAC | CTTTGATGAGCCTCAGAAGAA | GGAGTTCTGGTGCGTAGAATTC | GCGAATTGAAGCCAGTTGCAAGAAGAACGCCATGGCCCAC |
| CHYEX2.168 | ACGGCTGCTC(A/G)TTGTGCAGGA | TGTGCTGACGGCTGCT | TGTCTCACCTTCCTGCACA | CCATCGAATCGTCTATCAGTACTTTGTGCTGACGGCTGCTC |
| CLCNKBEX10.33 | GGCCACCTTG(G/C)TTCTCGCCTC | CCGCTCTGGCCACCTT | AGGTGATGGAGGCGAGA | GGTCTCAATTAGGCTTCATGTACTCCGCTCTGGCCACCTTG |
| CLCNKBEX15.64 | GCCAAGGACA(C/T)GCCACTGGAG | CCACACTGGCCAAGGA | CCTTGACCACCTCCTCCA | GCCGGTCATGTGCTCTGATATCACCACACTGGCCAAGGACA |
| CLCNKBEX4.19 | AATCCCGGAG(G/C)TGAAGACCAT | GGTTCTGGAATCCCGGA | CCGCCAACATGGTCTTC | GCGTGATATTCCATGATCTGAGGTTCTGGAATCCCGGAG |
| CLCNKBEX4.70 | GGATATCAAG(A/C)ACTTTGGGGC | TGGAGGACTACCTGGATATCAA | CCACTTTGGCCCCAAA | GCTGGTGATGGCTCTTCATATGGAGGACTACCTGGATATCAAG |
| COX2EX1.358 | CCAATTGTCA(T/G)ACGACTTGCA | CGGTTAGCGACCAATTGTC | GACGCTCACTGCAAGTCG | CGAACATCTGTCACAATGCGCTCGGTTAGCGACCAATTGTCA |
| COX2EX10.156 | ATGGTAGAAG(T/C)TGGAGCACCA | TTTGGTGAAACCATGGTAGAA | TCAAGGAGAATGGTGCTCC | GACTCTAGTGTCGTCTGATCTCTTTGGTGAAACCATGGTAGAAG |
| CYP11BIEX4.205 | AGGAGCACTT(T/G)GAGGCCTGGG | AAGGTGTGGAAGGAGCACT | ATGCAGTCCCAGGCCT | TCAGATGTTGTAATCGTGCGCAAGGTGTGGAAGGAGCACTT |
| CYP11BIEX5.107 | CGTGGCGGAG(C/G)TCCTGTTGAA | CAGTACACCAGCATCGTGG | AGTTCCGCATTCAACAGG | GCGTCGGCTTCATGCGATATTACACCAGCATCGTGGCGGAG |
| CYP11B2EX3.152 | CAGGCCCTGA(A/G)GAAGAAGGTG | GCAGTGGCCAGGGACT | CGTTCTGCAGCACCTTCTT | ATGCACGATCCTCTACATTGGGACTTCTCCCAGGCCCTGA |
| CYP11B2EX6.91 | GTGCAGCAGA(T/C)CCTGCGCCAG | CCCGACGTGCAGCAG | GCTCTCCTGGCGCAG | CTTACCCATGATTAGCGCAGGGAACCCCGACGTGCAGCAGA |
| CYPP11B2EX7.65 | GAGCGAGTGG(T/C)GAGCTCAGAC | GCTCTACCCTGTGGGTCTGT | TGAAGCACCAAGTCTGAGCT | GCCGATGGTGCGTCTACTATGTCTGTTTTTGGAGCGAGTGG |
| DBHEX3.153 | GCCCTCAGAC(G/A)CGTGCACCAT | CCGGAGTTGCCCTCAGA | GGACCTCCATGGTGCAC | TGGCAGGTTGTGACTCTCTCAACCGGAGTTGCCCTCAGAC |
| DBHEX4.132 | GATGAAACCC(G/A)ACCGCCTCAA | GCGACTCCAAGATGAAACC | GGCAGTAGTTGAGGCGG | TATGATTATTGAGTGCGGCCTGCGACTCCAAGATGAAACCC |
| DBHEX5.39 | AGCCGGCCTT(G/T)CCTTCGGGGG | CCAGAGGAAGCCGGC | CCTGGACCCCCGAAG | TCAGATCGTCTTGCTGTCGAACCCAGAGGAAGCCGGCCTT |
| DDIR.122 | CTCAGAGGAC(A/G)ACCCCGAGTA | TGACCCCTATTCCCTGCT | CTCTGACAAAATCAAGTTC | TTTGAGATTTGTCGAGAGCCACTGACCCCTATTCCCTGCTT |
| EDNRBEX3.144 | GATATAATTA(C/T)GATGGACTAC | CTGAAGCCATAGGTTTTGATATAAT | GCAGATAACTTCCTTTGTAGTCCA | GCCTGCTGTGGCTGTATATCAGATAACTTCCTTTGTAGTCCATC |
| ELAM1.77 | GACTTTCTGC(C/T)GCTGGACTCT | CCTTGGTAGCTGGACTTTCTG | GTCAGGAGGGAGAGTCCAG | GATCACTGTGGTCCCTGTCTGTAGCTGGACTTTCTGC |
| ELAM1EX5.197 | TTGGGACAAC(G/A)AGAAGCCAAC | CATCTGGGAATTGGGACAA | TCTACCTTTACACGTTGGCTTC | TATGAGTGTTGCGCTATGCCTCATCTGGGAATTGGGACAAC |
| ELAM1EX7.200 | GTGGGACAAC(G/C)AGAAGCCCAC | CACAGGGGAGTGGGACA | CCTTCACATGTGGGCTTC | GCGTCGCTGTCGTGTACTATCCACAGGGGAGTGGGACAAC |
| cNOS.78 | CCCCAGATGA(T/G)CCCCCAGAAC | TGCAGGCCCCAGATG | CAGAAGGAAGAGTTCTGGGG | ATACGGGATGATGAGCATACTGCTGCAGGCCCCAGATGA |
| ETIEX5.90 | TGAAAGGCAA(G/T)CCCTCCAGAG | TCCCAAGCTGAAAGGCA | CACATAACGCTCTCTGGAGG | TACATGACTTGCCCTGCTGTTTCATGATCCCAAGCTGAAAGGCAA |
| GALNREX1.327 | GCACGCAGCC(G/C)CTCCGGGAGC | CAGGTGCAGCACGCA | TCCCTGGCTCCCGGA | ACGATGAGCAGGGATCACTAACAGGTGCAGCACGCAGCC |
| GALNREX1.553 | TCAGAAGGTC(G/C)CGGCGCAAAG | CCCACCCTCTCTCAGAAGGT | CACCGTCTTTGCGCC | ATCTGAGAGCTAGTCGGCATCCACCCTCTCTCAGAAGGTC |
| GGREX9.29 | AACATGGGCT(T/G)CTGGTGGATC | AGCAATGACAACATGGGC | CCGCAGGATCCACCA | GGTGACTATTCGGCTGCTCTACCAGCAATGACAACATGGGCT |
| GLUT2EX1.137 | AGCACTAATT(C/A)TCTGTGGAGC | CTAAACAGAAACACCACAGCAC | ACTGCACTCTGCTCCACAG | TAGCTGTGTTGACATCTGGCACAGAAACACCACAGCACTAATT |
| GLUT2EX1.164 | CAGTGTGCCT(T/C)CCATGCTCCA | GCAGAGTGCAGTGTGCC | GCTGTGCTGTGGAGCATG | TGCTTAGTTGTGAGTCGCCAGAGCAGAGTGCAGTGTGCCT |
| GLUT4EX3.112 | GGCACCCTCA(C/G)CACCCTCTGG | CCCTCCAGGCACCCTC | AGAGGGCCCAGAGGGT | CTCACGACTGGGCTGATGATTCCATCCCTCCAGGCACCCTCA |
| GMP-140.105 | TTTCTCTTGT(A/G)ACAATGGCTT | TGGAGCGGTGGCTTCTA | CCCACCCATTATCAGACCTA | TGGCACAGTTTCCTGCTGGTGGCTCCACCTGTCATTTCTCTTGT |
| GMP-140.164 | CCACTGGTCA(A/C)CTACCGTGCC | AAGAGAATGGCCACTGGTC | GCAGGTTGGCACGGTA | GCTGGGTGTGATCCTCTCTACAAGAGAATGGCCACTGGTCA |
| GMP-140.25 | TACTCCAGGG(T/G)TGCAATGTCC | GCATCACTTCCTACTCCAGG | TGAGGGCTGGACATTGC | GGTGACAGTGTATTATCTGCATCACTTCCTACTCCAGGG |
| GMP-140.30 | GAAGCCCCCC(G/A)TGAAGGAACC | CAGCACCTGGAAGCCC | ACACAGTCCATGGTTCCTTC | GATCTGTTCAAAGTGATGGCGTCAGCACCTGGAAGCCCCCA |
| GNB3EX10.144 | CATCATCTGC(G/A)GCATCACGTC | CCCACGAGAGCATCATCTG | CACTGAGGGAGAAGGCCA | TATCTTATTCTCGACGCGGCTCCCACGAGAGCATCATCTGC |
| GSY1EX16.210 | CATCCGTGCA(C/G)CAGAGTGGCC | GCGCAACATCCGTGC | TGCAGGACGCTCGGC | CCTGTCTACCATGCAGTAATCGGCGCAACATCCGTGCA |
| GSY1EX3.117 | GGAGCGCTGG(A/G)AGGGAGAGCT | GGCCCTGGAGCGCTG | GGTATCCCAGAGCTCTCCC | TATATGCAGTGGTGTTCGCCTATCCCAGAGCTCTCCCT |
| GSY1EX8.43 | AACGGCAGCG(A/G)GCAGACAGTG | GCAGGTGAACGGCAGC | AAGAAGGCAACCACTGTCTG | GACGCGGGTGCTCATCATATCTGCGCAGGTGAACGGCAGCG |
| HUMGUANCYC.2905 | ATGTTGGGCT(C/G)AGGACCCAGC | TGGAGCGATGTTGGGC | CGCTCAGTGGGTCC | GCTGGGCATGTGTACTACTCTGATGGAGCGATGTTGGGCT |
| ICAM1.117 | TTCCCTGGAC(G/A)GGCTGTTCCC | CGTGGTCTGTTCCCTGGA | CCTCCGAGACTGGGAACA | CTGTCAATGCGTCTGCTCTAGACCGTGGTCTGTTCCCTGGAC |
| ICAM1EX1.683 | TTGCAACCTC(A/C)GCCTCGCTAT | TGCTACTCAGAGTTGCAACCT | GGGAGCCATAGCGAGG | GTCTCGCTTCGTGAGTGCAGCTACTCAGAGTTGCAACCTC |
| ICAM1EX2.115 | GACCAGCCCA(A/T)GTTGTTGGGC | CACCTCCTGTGACCAGCC | GGGTCTCATGCCCAACAA | ACGCACACTGATAACTATGCACCTCCTGTGACCAGCCCA |
| ICAM1EX6.254 | GGTCACCCGC(G/A)AGGTGACCGT | AGGGGAGGTCACCCG | AGCACATTCACGGTCACC | GTGCTGGGTTCGCATTCATCGCACATTCACGGTCACCT |
| ICAM1EX6.39 | GATGGCCCCC(G/A)ACTGGACGAG | TTTTCCAGATGGCCCC | GACAATCCCTCTCGTCCAG | CCAATAGGTGCTCACGTCATGTGTTTTTCCAGATGGCCCCC |
| ICAM2EX2.63 | AAAGAAGCTG(G/A)CGGTTGAGCC | CGTGAGGCCAAAGAAGCT | CCCTTTGGGCTCAACC | TTGGCTCATTTGCATGGCGCCACGTGAGGCCAAAGAAGCTG |
| IRS-2.AA1057 | CAGGGCCCGG(G/A)CGCCGCCTCA | GTTGCCACCGCCCAG | CAACGATGAGGCGGC | TGCTCGCTTGTGATCGACTGTTGCCACCGCCCAGGGCCCGG |
| KLKEX3.523 | GTTGCCCACC(C/G)AGGAACCCGA | TCGTGGAGTTGCCCAC | CCCCACTTCGGGTTCC | CCTGTCGCGCCTGATAGAATGTCGTGGAGTTGCCCACC |
| KLKEX4.110 | GTCCAGAAGG(T/A)GACAGACTTC | AAGCCCACGTCCAGAAG | CGACACACAGCATGAAGTCTG | ACGCAATATCGGCCATCGTGGCAAAAAAGCCCACGTCCAGAAGG |
| LAM1.103 | CCAGTGTCAG(T/C)TTGGTAAGTC | TGGGCCCCAGTGTCA | GCAAAGAAAGGAAAGAGACTTACC | CTGTGCCCTGCTCTGATGATTACTATGGGCCCCAGTGTCAG |
| LPL.177 | TATGAGATCA(A/G)TAAAGTCAGA | CAACAATCTGGGCTATGAGATC | TGCTTCTTTTGGCTCGACTT | GTGCCTGTTGACATATAGTGACAATCTGGGCTATGAGATCA |
| LPL.98 | AATAAGAAGT(C/G)AGGCTGGTGA | CCATGACAAGTCTCTGAATAAGAA | CCAGAATGCTCACCAGCC | CCTGTAGTGCAGTCTCCTGACGCATGACAAGTCTCTGAATAAGAAGT |
| mACEEX13_R.NA | TGCTGGTCCC(C/T)AGCCAGGAGG | GCACCCTCTGCTGGTCC | TGACTGTCACCTGTTGGGA | CACTCACTGGCACGGTATAGTGTTGGGATGCCTCCTGGCT |
| MRLEX2.545 | CATGCGCGCC(A/G)TTGTTAAAAG | GGTGGCGTCATGCGC | CACATGATAGGGCTTTTAACAAT | GGAATGTCTGCCGTGCCATAATGGTGGCGTCATGCGCGCC |
| NAT2.346 | CAGGTGACCA(T/C)TGACGGCAGG | CCTTCTCCTGCAGGTGACC | ACAATGTAATTCCTGCCGTC | CTGTGAGTGATGTACGCTCCTTCTCCTGCAGGTGACCA |
| NETEX11.123 | AGTCCTGCCT(T/G)CCTCCTGGTG | GAAGTTCGTCAGTCCTGCC | CCCTGCAGACACTACACACC | GCGTGCGGTTCATCTGCATTCTGGAAGTTCGTCAGTCCTGCCT |
| NETEX12.81 | CTACGACGAC(T/C)ACATCTTCCC | GCCACTCACCTACGACGA | CCAGGGCGGGAAGATG | CGGCTGGGTAGCATCATCTAAAGCCACTCACCTACGACGAC |
| NETEX5.121 | AATGGCATCA(A/C)TGCCTACCTG | GAGCCTCCAATGGCATC | GTCGATGTGCAGGTAGGC | GCATGAAGTTCCATAATCGCGAGCCTCCAATGGCATCA |
| NETEX7.112 | TGGTTACATG(G/C)CCCATGAACA | TCTTCTCCATCCTTGGTTACA | TGTTGACCTTGTGTTCATGG | CAGTGACATGCCGCTCAGTACATCTTCTCCATCCTTGGTTACATG |
| NETEX7.131 | CACAAGGTCA(A/G)CATTGAGGAT | GCCCATGAACACAAGGTC | TGTGGCCACATCCTCAAT | CGGCAATATGATGATAGGTCCCCATGAACACAAGGTCA |
| NETEX7.73 | CACCAGCTTC(G/C)TCTCTGGGTT | AGATGGCGAACCCAGAG | AGATGGCGAACCCAGAG | CCTGGTATGACATGGAGCCTCAGCATCAACTGTATCACCAGCTTC |
| NETEX9.157 | TGCATAACCA(A/G)GGTGAGTAGG | GCCCAGCCCCTACTCAC | GCCCAGCCCCTACTCAC | CCAACGATGCTACTGAGTCACGCCCTGTTCTGCATAACCA |
| OB.160 | GATCAATGAC(A/G)TTTCACACAC | AATTGTCACCAGGATCAATGA | ACTCTCCTTACCGTGTGTGAA | CATTGCACCCACTGAGATGGATTGTCACCAGGATCAATGAC |
| OB-R.174 | GTAATTTTCC(A/G)GTCACCTCTA | TCACATCTGGTGGAGTAATTTTC | GCTGAACTGACATTAGAGGTGA | CACGGATCTGCCGCTAGAATCATCTGGTGGAGTAATTTTCC |
| PGISEX1.396 | GGGAGCAGGG(T/G)TTCTCCCAGA | GCTGCGGGGAGCAGG | GGGCGCTCTGGGAGA | CGAACACATGCGGCTGGATAAGCTGCGGGGAGCAGGG |
| PLA2AEX2.42 | GCCGCCGCCG(A/C)CAGCGGCATC | CTTGCAGTGGCCGCC | AGGGCTGATGCCGCT | AGATAGAGTCGATGCCAGCTTTGCAGTGGCCGCCGCCG |
| PLA2AEX3.104 | TGCTGGACAA(C/A)CCGTACACCC | TGGACAGCTGTAAATTTCTGCT | ATGAATAGGTGTGGGTGTACG | TGCCTCATTGTGACTCATGGACAGCTGTAAATTTCTGCTGGACAA |
| PNMTEX3.181 | GGAGGCTGTG(A/T)GCCCAGATCT | CCTTCTGCTTGGAGGCTGT | AGCTGGCAAGATCTGGG | TGTGAGCTTGTTACTACGGCTGCCTTCTGCTTGGAGGCTGTG |
| PNMTEX3.251 | GGGGGGGACC(T/A)CCTCCTCATC | GCTGAGGCCTGGGGG | CCAGGTACCACGACTCCTC | TGTGAATATGTGTGCCACTGAGGCCTGGGGGGCACC |
| PNMTEX3.269 | ATCGGGGCCC(T/A)GGAGGAGTCG | GCTGAGGCCTGGGGG | CCAGGTACCACGACTCCTC | TGAGACTATTTAGGCTGTGCTCCTCCTCATCGGGGCCC |
| PON1.584 | CCCTACTTAC(A/G)ATCCTGGGAG | TCACTATTTTCTTGACCCCTACTT | CCCAAATACATCTCCCAGGA | GATCGCAGTTCAGAGCGCATATTTTCTTGACCCCTACTTAC |
| PON2.949 | AACATTCTAT(G/C)TGAGAAGCCT | CCGCATCCAGAACATTCTA | CATAAACTGTAGTCACTGTAGGCTTCT | CAGTCTCGTGGATAGCACTCGTTCTCCGCATCCAGAACATTCTAT |
| SCNNIB.222 | CACCAACTTT(G/A)GCTTCCAGCC | GGAGGCCCACACCAACTT | CCGTGTCAGGCTGGAAG | GACTGGGATTACATGCTATGGAGGCCCACACCAACTTT |
| SCNNIB.238 | CAGCCTGACA(C/T)GGCCCCCCGC | TTGGCTTCCAGCCTGAC | GTGTTGGGGCTGCGG | CACTCCGATGGCGAGATGAATTTGGCTTCCAGCCTGACA |
| SCNNIB.AA442 | ACCTGCATTG(G/T)CATGTGCAAG | CGCAGAGAGAGACCTGCATT | GCAGGACTCCTTGCACAT | GCACGTCTGTCGATCTATACAGAGAGAGACCTGCATTG |
| SCNNIG.172 | GGCTCCCCCA(T/C)GTCCAGAAGC | GGAAACAGGCTCCCCC | ACGGGGAGCTTCTGGA | AGCCAAGTGCAGGCGTACATCCTGGAAACAGGCTCCCCCA |
| SCNNIG.21 | TTCTGTCCAA(C/A)TTCGGTGGCC | CAGTATTGAGATGCTTCTGTCCA | CCAGCTGGCCACCGA | TCCTCTCGTTGGATGTGAGCCAGTATTGAGATGCTTCTGTCCAA |
| SCNNIGEX1.236 | GTCGTGGCCC(G/T)CTCCGGGCGG | CGTTGTGAAGTCGTGGCC | CTGAGACCGCCCGGA | CAGTGACGTGAGTGCCATCTGTTGTGAAGTCGTGGCCC |
| SCNNIGEX2.219 | GGTGTCCCGC(G/T)GCCGTCTGCG | GCATCGTGGTGTCCCG | GGAGGCGGCGCAGAC | CTCAGCAGTTAGCAGCGCATCGCATCGTGGTGTCCCGC |
| SCNNIGEX3.259 | GCGGAAAGTC(G/A)GCGGTAGCAT | GGGAGGAAGCGGAAAGT | GAAGCCTTGTGAATGATGCT | CTTATGGCGCTGTCGGCTATCAGGGAGGAGCGGAAAGTC |
| TBXASEX11.88 | CCCCGCAGGC(G/A)CTGTGCTAGA | CGAGGTGCTGGGGCA | ACGGCCATCTCTAGCACA | GATATGCGTTACGTGAGTCTCGGCCATCTCTAGCACAG |
| TBXASEX9.276 | TGCCACCTAC(C/G)TACTGGCCAC | CACACTTTCTTTTGCCACCT | AGGGTTGGTGGCCAGT | CAACAACTGCGCGACGATGAAACACACTTTCTTTTGCCACCTAC |
| TGF-B1.75 | CTCATGGCCA(C/T)CCCGCTGGAG | TCCTGCTTCTCATGGCC | GGCCCTCTCCAGCGG | TTGTGCATTGTTGGACGCCCCTTTCCTGCTTCTCATGGCCA |
| TRHREX1.56 | GCAGAACTTA(G/C)ATGATAAGCA | CAGGTACTAGAGTTTCTGCAGAACTT | GGCTTTGTCGTTGCTTATCA | AGCAGTAATGACAGCGTGCAAGGTACTAGAGTTTCTGCAGAACTTA |

References:
1. Singer-Sam & Riggs, Methods Enzymol. 225:344-351 (1993).
2. Greenwood & Burke, Genome Res. 6:336-348 (1996).
3. Halushka et al., Pattern of single nucleotide polymorphisms in human genes. Nature Genet. (Submitted).
4. Risch & Merikangas, Science 273:1516-1517 (1996).
5. Collins et al., Science 278:1580-1581 (1997).
6. Collins et al., Science 282:682-689 (1998).
7. Chakravarti, Nature Genet. 21:56-60 (1999).
8. Chee et al., Science 274:610-614 (1996).
9. Wang et al., Science 280:1077-1082 (1998); http://wwwoenome.wi.mit.edu/SNP/human/index.html.
10. Lipshutz et al., Nature Genet. 21:20-24 (1999).
11. Shoemaker et al., Nature Genet. 14:450-456 (1996).
12. Giaever et al., Nature Genet. 21:278-283 (1999).
13. Pastinen et al., Clin. Chem. 42:1391-1397(1996).
14. Pastinen et al., Genome Res. 7:606-614 (1997).
15. Pastinen et al., Hum. Mol. Genet. 7:1453-1462 (1998).
16. Nikiforov et al., PCR Methods and Applications 3:285-291 (1994).
17. Nikiforov et al., Nucleic Acids Res. 22:4167-4175 (1994).
18. Head et al., Nucleic Acids Res. 25:5065-5071 (1997).
19. Tobe et al., Nucleic Acids Res. 24:3728-3732 (1996).
20. Delahunty et al., Am. J. Hum. Genet. 58:1239-1246 (1996).
21. Chen et al., Genome Res. 8:549-556 (1998).
22. Lyamichev et al., Nature Biotech. 17:292-296 (1999).
23. Newton et al., Nucleic Acids Res. 17:2503-2516 (1989).
24. Lo et al., Nucleic Acids Res. 19:3561-3567 (1991).
25. Zhang et al., Proc. Natl. Acad. Sci. USA 89:5847-5851 (1992).

While this invention has been particularly shown and described with references to preferred embodiments thereof, it will be understood by those skilled in the art that various changes in form and details may be made therein without departing from the scope of the invention encompassed by the appended claims.

1. A method of genotyping a nucleic acid sample at one or more loci, comprising the steps of:
(a) combining a nucleic acid sample comprising a nucleic acid molecule with one or more locus-specific tagged oligonucleotides under conditions suitable for hybridization of the nucleic acid molecule in the nucleic acid sample to one or more locus-specific tagged oligonucleotides, wherein each locus-specific tagged oligonucleotide comprises (a) a nucleotide sequence capable of hybridizing to a complementary sequence in an oligonucleotide tag and (b) a nucleotide sequence complementary to the nucleic acid molecule in the nucleic acid sample which terminates one nucleotide 5′ of a nucleotide locus to be queried in the nucleic acid molecule in the nucleic acid sample, thereby creating an amplification product-locus-specific tagged oligonucleotide complex;
(b) subjecting the complex to a single base extension reaction in the presence of two or more labeled ddNTPs, wherein the reaction results in the addition of a labeled ddNTP to the locus-specific tagged oligonucleotide, and wherein each type of ddNTP has a label that can be distinguished from the label of the other three types of ddNTPs;
(c) contacting the complex with an oligonucleotide array comprising one or more oligonucleotide tags fixed to a solid substrate under suitable hybridization conditions, wherein each oligonucleotide tag comprises a unique arbitrary sequence complementary and of sufficient length to hybridize to a complementary sequence in a locus-specific tagged oligonucleotide, whereby the complex hybridizes to a specific oligonucleotide tag on the array; and assaying the array to determine the labeled ddNTPs present in the complex hybridized to one or more oligonucleotide tags, thereby determining the genotype of the queried nucleotide locus.

2. A method to aid in determining a ratio of alleles at a polymorphic locus in a sample, comprising the steps of:
(a) using a pair of primers to amplify a region of a nucleic acid in a sample, wherein the region comprises a polymorphic locus, whereby an amplified DNA product is formed;
(b) labeling an extension primer by a single base extension reaction to form a labeled extension primer, wherein the amplified DNA product is used as a template, wherein the extension primer comprises a 3′ portion and a 5′ portion, wherein the 3′ portion is complementary to the amplified DNA product and terminates one nucleotide 5′ to the polymorphic locus, wherein the 5′ portion is not complementary to the amplified DNA product, whereby a labeled dideoxynucleotide which is complementary to the polymorphic locus is coupled to the 3′ end of the extension primer by the single base extension reaction, wherein the single base extension reaction is carried out in the presence of two or more labeled dideoxynucleotides, and wherein each type of dideoxynucleotide bears a distinct label; and
(c) hybridizing the 5′ portion of the extension primer to one or more probes complementary to the 5′ portion of the extension primer which are immobilized to known locations on a solid support,
thereby determining the ratio of alleles at a polymorphic locus in a sample.

3. The method of claim 2 wherein two complementary strands of the amplified DNA product are present in the single base extension reaction.

4. The method of claim 2 wherein two complementary strands of the amplified DNA product are used as templates in the step of labeling.

5. The method of claim 2 wherein the label is a fluorescent label.

6. The method of claim 2 wherein the label is a radiolabel.

7. The method of claim 2 wherein the label is an enzyme label.

8. The method of claim 2 wherein the label is an antigenic label.

9. The method of claim 2 wherein the label is an affinity binding partner.

10. The method of claim 2 further comprising the step of:
(d) optically detecting a fluorescent label on the solid support.

11. (Cancelled)

12. The method of claim 2 wherein the step of labeling employs four distinct dideoxynucleotides bearing distinct labels.

13. The method of claim 2 further comprising the steps of:
(d) comparing quantities of a first and a second label at a location on the solid support; and
(e) determining the ratio of nucleotides present at the polymorphic locus in the sample.

14. The method of claim 13 wherein the ratio of nucleotides present at two or more polymorphic loci is determined simultaneously.

15. The method of claim 2 wherein the sample comprises DNA from two or more individuals.

16. The method of claim 15 wherein the ratio of nucleotides present at two or more polymorphic loci is determined simultaneously.

17. The method of claim 2 wherein the solid support is selected from the group consisting of beads, microtiter plates, and oligonucleotide arrays.

18. A method to aid in determining a ratio of alleles at a polymorphic locus, comprising the steps of:
(a) labeling an extension primer by a single base extension reaction to form a labeled extension primer, using a DNA molecule containing a polymorphic locus as a template, wherein the extension primer comprises a ₃′ portion and a 5′ portion, wherein the 3′ portion is complementary to the DNA molecule and terminates one nucleotide 5′ to a polymorphic locus, wherein the 5′ portion is not complementary to the DNA molecule, whereby a labeled dideoxynucleotide which is complementary to the polymorphic locus is coupled to the 3′ end of the extension primer, wherein the reaction is carried out in the presence of one or more dideoxynucleotides and wherein each type of dideoxynucleotide bears a distinct label; and
(b) hybridizing the 5′ portion of the extension primer to one or more probes complementary to the 5′ portion of the extension primer which are immobilized to known locations on a solid supports
thereby aiding in the determination of a ratio of alleles at a polymorphic locus.

19. The method of claim 18 wherein two complementary strands of the DNA molecule are present in the single base extension reaction.

20. The method of claim 19 wherein each complementary strand of the DNA molecule is used as a template to label an extension primer.

21. The method of claim 18 wherein the label is a fluorescent label.

22. The method of claim 18 wherein the label is a radiolabel.

23. The method of claim 18 wherein the label is an enzyme label.

24. The method of claim 18 wherein the label is an antigenic label.

25. The method of claim 18 wherein the label is an affinity binding partner.

26. The method of claim 18 further comprising the step of:
(c) optically detecting a fluorescent label on the solid support.

27. The method of claim 18 further comprising the steps of:
(c) comparing quantities of a first and a second label at a location on the solid support; and
(d) determining the ratio of nucleotides present at the polymorphic locus in the sample.

28. The method of claim 27 wherein the ratio of nucleotides present at two or more polymorphic loci is determined simultaneously.

29. The method of claim 18 wherein the sample comprises DNA from two or more individuals.

30. The method of claim 26 wherein the ratio of nucleotides present at two or more polymorphic loci is determined simultaneously.

31. The method of claim 18 wherein the step of labeling employs at least two distinct dideoxynucleotides bearing distinct labels.

32. The method of claim 18 wherein the step of labeling employs four distinct dideoxynucleotides bearing distinct labels.

33. The method of any one of claim 1, wherein the oligonucleotide array comprises at least 10 oligonucleotide tags fixed to a solid substrate.

34. The method of any one of claim 1, wherein the oligonucleotide array comprises at least 100 oligonucleotide tags fixed to a solid substrate.

35. The method of any one of claim 1, wherein the oligonucleotide array comprises at least 1000 oligonucleotide tags fixed to a solid substrate.

36. The method of any one of claim 2, wherein the oligonucleotide array comprises at least 10 oligonucleotide tags fixed to a solid substrate.

37. The method of any one of claim 2, wherein the oligonucleotide array comprises at least 100 oligonucleotide tags fixed to a solid substrate.

38. The method of any one of claim 2, wherein the oligonucleotide array comprises at least 1000 oligonucleotide tags fixed to a solid substrate.

39. The method of any one of claim 18, wherein the oligonucleotide array comprises at least 10 oligonucleotide tags fixed to a solid substrate.

40. The method of any one of claim 18, wherein the oligonucleotide array comprises at least 100 oligonucleotide tags fixed to a solid substrate.

41. The method of any one of claim 18, wherein the oligonucleotide array comprises at least 1000 oligonucleotide tags fixed to a solid substrate.
